(19) United States
(12) Patent Application Publication
(10) Pub. No.: US 2019/0185544 A1
(43) Pub. Date: Jun. 20, 2019

(54) COMPOSITIONS AND METHODS OF MODULATING ABETA PROTEIN

(71) Applicants: DARRELL SAWMILLER, Land O Lakes, FL (US); HUAYAN HOU, Tampa, FL (US); JUN TAN, Tampa, FL (US)

(72) Inventors: DARRELL SAWMILLER, Land O Lakes, FL (US); HUAYAN HOU, Tampa, FL (US); JUN TAN, Tampa, FL (US)

(73) Assignee: University of South Florida, Tampa, FL (US)

(21) Appl. No.: 16/180,461

(22) Filed: Nov. 5, 2018

Related U.S. Application Data
(63) Continuation-in-part of application No. PCT/US2017/030826, filed on May 3, 2017.
(60) Provisional application No. 62/402,270, filed on Sep. 30, 2016.
(60) Provisional application No. 62/331,055, filed on May 3, 2016.

(51) Int. Cl. C07K 14/775; A61P 25/28

(52) U.S. Cl. CPC C07K 14/775; A61K 38/00; A61P 25/28

(57) ABSTRACT

Provided herein are recombinant polypeptides and formulations thereof that can modulate binding of an ApoE protein to an ApoE receptor and/or APP. Also provided herein are methods of administering the recombinant polypeptides and formulations thereof to a subject in need thereof. The subject in need thereof can have or be suspected of having a neurological disease or disorder.

### CROSS-REFERENCE TO RELATED APPLICATIONS

This application is a continuation-in-part application filed under 35 U.S.C. § 111(a) of Patent Cooperation Treaty Application Serial No.: PCT/US2017/030826, filed on May 3, 2017, entitled “COMPOSITIONS AND METHODS OF MODULATING ABETA PROTEIN,” the contents of which are incorporated by reference herein in its entirety.

Patent Cooperation Treaty Application Serial No.: PCT/US2017/030826 claims the benefit of and priority to U.S. Provisional Patent Application No. 62/331,055, filed on May 3, 2016, entitled “COMPOSITIONS AND METHODS OF MODULATING ABETA PROTEIN,” and U.S. Provisional Patent Application No. 62/402,270, filed on Sep. 30, 2016, entitled “COMPOSITIONS AND METHODS OF MODULATING ABETA PROTEIN,” the contents of which are incorporated by reference herein in its entirety.

### SEQUENCE LISTING

This application contains a sequence listing filed in electronic form as an ASCII.txt file entitled 292105-1690_ST25.txt, created on Oct. 30, 2018. The content of the sequence listing is incorporated herein in its entirety.

### BACKGROUND

Alzheimer's disease (AD) is an irreversible, progressive brain disorder that slowly destroys memory and thinking skills, and eventually the ability to carry out the simplest of tasks. AD is currently the sixth leading cause of death in the United States and ranks third in as the leading cause of death in the elderly. As such, there exists an urgent need for treatments for AD.

### SUMMARY

Described herein are compositions that can include a compound configured to reduce binding of an apoliprotein E (ApoE) protein to an ApoE receptor or to amyloid precursor protein (APP). In aspects the compound can specifically have the same sequence as the low-density lipoprotein receptor binding domain on the ApoE protein and bind to the ApoE receptor or APP, wherein the LDL receptor binding domain has a polypeptide sequence according to SEQ ID NO: 1, or a polypeptide sequence that is about 90-100% identical to SEQ ID NO: 1, or a fragment thereof having at least 5 contiguous amino acids. The compound can be an antibody or fragment thereof. In embodiments, the compound can be a small molecule. The compound can be a competitive inhibitor of ApoE for the ApoE receptor. The compound can be a recombinant polypeptide, wherein the recombinant polypeptide can include or be a polypeptide sequence according to SEQ ID NO: 1 or a polypeptide sequence that is about 90-100% identical to SEQ ID NO: 1 and at least 3 or at least 6 additional lysine residues operatively coupled to the polypeptide sequence according to SEQ ID NO: 1 or the polypeptide sequence that is about 90-100% identical SEQ ID NO: 1. In some aspects, at least 3 or at least 6 lysine additional residues can be operatively coupled to the N-terminus of the polypeptide sequence according to SEQ ID NO: 1 or the polypeptide sequence that is about 90-100% identical SEQ ID NO: 1. In some aspects, the at least 3 additional lysine residues are operatively coupled to the C-terminus of the polypeptide sequence according to SEQ ID NO: 1 or the polypeptide sequence that is about 90-100% identical SEQ ID NO: 1. In some aspects, the at least 3 additional lysine residues are operatively coupled between the N-terminus and C-terminus of the polypeptide sequence according to SEQ ID NO: 1 or the polypeptide sequence that is about 90-100% identical SEQ ID NO: 1. The compositions described here can further include a pharmaceutical carrier.

Also provided herein are recombinant polypeptides that can include or be a polypeptide sequence according to SEQ ID NO: 1 or a polypeptide sequence that is about 90-100% identical to SEQ ID NO: 1. The recombinant polypeptide can further include at least 3 or at least 6 additional lysine residues operatively coupled to the polypeptide sequence according to SEQ ID NO: 1 or the polypeptide sequence that is about 90-100% identical SEQ ID NO: 1. In some aspects, the at least 3 or at least 6 lysine additional residues are operatively coupled to the N-terminus of the polypeptide sequence according to SEQ ID NO: 1 or the polypeptide sequence that is about 90-100% identical SEQ ID NO: 1. In some aspects, the at least 3 or at least 6 additional lysine residues are operatively coupled to the C-terminus of the polypeptide sequence according to SEQ ID NO: 1 or the polypeptide sequence that is about 90-100% identical SEQ ID NO: 1. In some aspects, the at least 3 or at least 6 additional lysine residues are operatively coupled between the N-terminus and C-terminus of the polypeptide sequence according to SEQ ID NO: 1 or the polypeptide sequence that is about 90-100% identical SEQ ID NO: 1.

Also described herein are polynucleotide sequences that can be configured to encode a recombinant polypeptide as described anywhere herein.

Also provided herein are vectors that can include vectors that can include a nucleotide sequence configured to encode a recombinant polypeptide as described herein. The vector can be an expression vector configured to express the recombinant polypeptide in a host cell.

Also provided herein are cells that can contain and/or express a recombinant polypeptide as described herein.

Also provided herein are cells that can include and/or express a polynucleotide sequence configured to encode a recombinant polypeptide as described herein.

Also provided herein are cells that can include a vector as described herein.

Also provided herein are transgenic animals that can include and/or express a recombinant polypeptide as described herein, a polynucleotide as described herein, a vector as described herein, and/or a cell as described herein.

Also provided herein are methods that can include the step of administering an amount of a composition as described herein, a recombinant polypeptide as herein, a polynucleotide as described herein, a vector as described herein, and/or as cell as described herein to a subject. The methods can further include the step of measuring binding of an apolipoprotein E (ApoE) to an ApoE receptor. The methods can further include the step of measuring Aβ protein formation. The subject can have a neurologic disease or disorder. The subject can have Alzheimer's disease. The subject can have a cardiovascular disease or disorder.

Also provided herein are methods that can include the step of contacting a candidate compound with a recombinant polypeptide as described herein or an ApoE protein; and measuring binding of the recombinant polypeptide as described herein or the ApoE protein to an ApoE receptor present on the surface of a cell. In some aspects, the binding of the recombinant polypeptide as described herein or the ApoE protein to ApoE receptor can be measured by measuring ApoE receptor activity. In some aspects, the ApoE receptor can be a low-density lipoprotein receptor. In other aspects, the ApoE receptor can be amyloid precursor protein (APP).

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a graph demonstrating in vitro amyloid β protein (Aβ) in CHO/APPwt cells in the presence and absence of different ApoE isoforms.

FIG. 2 shows a graph demonstrating in vitro amyloid β protein (Aβ) in CHO/APPwt cells in the presence and absence of ApoE LDL receptor binding domain peptide (SEQ ID NO.: 1).

FIG. 3 shows a graph demonstrating in vitro amyloid β protein (Aβ) in CHO/APPwt cells in the presence and absence of structurally modified ApoE LDL receptor binding domain peptide (ApoE LDLR).

FIG. 4 shows a graph demonstrating in vitro amyloid β protein (Aβ) in CHO/APPwt cells treated with Human plasma ApoE.

FIG. 5 shows a graph demonstrating in vitro amyloid β protein (Aβ) in CHO/APPwt cells treated with recombinant human ApoE3, 4 (rec hu ApoE3, 4) or Control (Ctl).

FIG. 6 shows a graph demonstrating in vitro amyloid β protein (Aβ) in CHO/APPwt cells treated with human ApoE LDL receptor binding domain (ApoE LDLR) (SEQ ID NO: 1).

FIG. 7 shows a graph demonstrating in vitro amyloid β protein (Aβ) in CHO/APPwt cells pre-treated with 3KApoE LDLR, human plasma, recombinant ApoE (rec ApoE), or FlagApoE LDLR.

FIG. 8 shows a graph demonstrating in vitro amyloid β protein (Aβ) in CHO/APPwt cells pre-treated with 3KApoE LDLR/ApoE LDLR, human plasma ApoE3 & 4, ApoB100 and LDL.

FIGS. 9A-9F show data demonstrating human plasma LDL, ApoB100 and human recombinant ApoE3 &4 proteins markedly promote amyloid β protein (Aβ) production. CHO cells overexpressing human wild-type APP (CHO/APPwt) cells were cultured in 96-well plate at 2×10⁴/well in Dulbecco's modified Eagle's medium (DMEM) with 10% fetal bovine serum (FBS), 1 mM sodium pyruvate and 100 U/mL of penicillin/streptomycin for 18 hours. These cells were further treated with human plasma LDL (hu LDL) (FIGS. 9A and 9C), human plasma ApoB100 (hu ApoB100) (FIGS. 9B and 9D), human recombinant ApoE3, 4 protein (hu rec ApoE3 or 4) or PBS (Ctrl) (FIGS. 9E and 9F) at various concentrations in serum-free DMEM as indicated for 2 and 16 hours (h), followed by analysis of conditioned media and cell lysates by Aβ1-40, 42 ELISA, 82E1 western blotting (WB) analysis and protein assay, respectively. 82E1 WB analyses as shown below the Aβ ELISA result histograms. The Aβ ELISA results are representative of three independent experiments with each condition triplicated and presented as mean±SD.

FIGS. 10A-10C show data demonstrating ApoE LDL receptor binding domain peptide (ApoE-LBDP) treatment significantly increases Aβ generation, which is structurally dependable. CHO/APPwt cells were cultured in 96-well plate at 2×10⁴/well in DMEM with 10% FBS for overnight and then serum-free DMEM in the presence or absence of ApoE LDL receptor binding domain peptide [ApoE-LBDP, LRVRLASHLRKLRKRLLRDA (residues 133-152)] as indicated for 2 and 16 hours (h). The conditioned media and cell lysates were analyzed by Aβ1-40, 42 ELISA (FIG. 10A), 82E1 WB analysis (FIG. 10B) and protein assay, respectively. In addition, (FIG. 10C) CHO/APPwt cells were cultured in 96-well plate (2×104/well) in serum-free DMEM and treated with FlagApoE-LBDP, BiotinApoE-LBDP and 3 lysineApoE-LBDP (3KApoE-LBDP) in comparing to ApoE-LBDP at indicated doses for 2 hours (h). The conditioned media and cell lysates were prepared, and subjected to Aβ1-40, 42 ELISA (**P<0.01; ***P<0.001 when compared to ApoE-LBDP) and protein assay. These results are representative of three independent experiments with each condition triplicated. Note: KKK-LRVRLASHLRKLRKRLLRDA; DYKDDDDK-LRVRLASHLRKLRKRLLRDA; Biotin-LRVRLASHLRKLRKRLLRDA.

FIGS. 11A-11D show data demonstrating 6KApoE markedly inhibits Aβ generation induced by human recombinant ApoE4 protein (hu rec ApoE4). CHO/APPwt cells were cultured in 96-well plate (2×104/well) in serum-free medium and pre-treated with PBS (Ctrl), 3 lysine (3K), 6K lysine (6K), 3KApoE-LBDP (3KApoE), 6KApoE-LBDP (6KApoE), 7KApoE-LBDP (7KApoE), 8KApoE-LBDP (8KApoE), 9KApoE-LBDP (9KApoE) peptide at 10 μM for 15 minutes and went to treatment with or without human recombinant ApoE4 protein (hu rec ApoE4, 10 μg/mL) for 2 hours. The conditioned media and cell lysates were prepared, and subjected to Aβ1-40, 42 ELISA and protein assay. Further, CHO/APPwt cells were cultured in 96-well plate (2×10⁴/well) in serum-free DMEM and pre-treated with 6KApoE for 15 minutes at various doses as indicated and then went to treatment with human recombinant ApoE4 protein (hu rec ApoE4) for 2 and 16 hours. The conditioned media and cell lysates were prepared, and subjected to Aβ1-40, 42 ELISA (FIG. 11C), 82E1 WB analysis (FIG. 11D) and protein assay, respectively. The Aβ1-40, 42 ELISA results are representative of three independent experiments with each condition triplicated and presented as mean±SD.

FIGS. 12A-12F show data demonstrating LDL receptor is in part responsive for 6KApoE inhibition of Aβ generation induced by human recombinant ApoE4 protein. CHO/APPwt cells were cultured in 96-well plate (2×10⁴/well) in serum-free medium and pre-treated with 6KApoE at 1 μM for 15 minutes and went treatment with an agonist anti-LDLR antibody (anti-LDLR Ab) from 0-2.5 μg/mL for 2 hours. The conditioned media and cell lysates were prepared, and subjected to Aβ1-40, 42 ELISA (FIG. 12A), 82E1 WB analysis (FIG. 12B) and protein assay. Hamster LDLR siRNA and negative control were ordered from Thermo Fisher Scientific, transfection was performed following the protocol of lipofectamine RNAiMAX reagent provided by Thermo Fisher Scientific. Briefly, CHO APPwt cells were plated in 24 well plate 1×10⁵ cells/well for overnight, siRNAs were transfected at 10 nM as indicated, 24 hours after these cells were washed and treated with human recombinant ApoE4 protein (hu rec ApoE4) at 10 μg/mL for 2 hours (h) in the presence or absence of 15-minute pre-treatment of 1 μM 6KApoE. The conditioned media and cell lysates were prepared, and subjected to Aβ1-40, 42 ELISA (FIG. 12C), anti-LDLR antibody WB evaluation (FIG. 12D) and protein assay. CHO/IdlA7 (IdlA7) and CHO wild-type (CHO) cells were provided by Dr. Monty Krieger (Massachusetts Institute of Technology, Cambridge, Mass.). The two cells were cultured in Ham's F-12 medium supplemented with 5% FBS, 2 mM L-glutamine. The cells were plated into 24-well plate at 1×10⁵ each well the day before transfection. PCMV6-APP695 (OriGene Technologies, Inc. Rockville, Md.) was transfected to these cells using Lipofectamine® 3000 Transfection Reagent (Thermofisher Scientific) according to the instructions. Twenty-four hours after transfection, these cells were washed and treated with human recombinant ApoE4 protein (hu rec ApoE4) at 10 μg/mL for 2 hours (h) in the presence or absence of 15-minute pre-treatment of 1 μM 6KApoE. The conditioned media and cell lysates were prepared, and subjected to Aβ1-40 ELISA (FIG. 12E), anti-LDLR antibody and APP processing WB evaluation (FIG. 12F), and protein assay. For Aβ ELISA, these results are representative of three independent experiments with each condition triplicated and presented as mean±SD.

FIGS. 13A-13B show graphs that can demonstrate amounts of Aβ₁₋₄₀ (FIG. 13A) and Aβ₁₋₄₂ (FIG. 13B) as measured by enzyme linked immunosorbent assay (ELISA) the brain of control (5×FAD) and treated (5×FAD/6KApoEp) mice after peripheral administration of 6KApoEp treatment.

FIGS. 14A-14B show graphs that can demonstrate amounts of Aβ₁₋₄₀ (FIG. 14A) and Aβ₁₋₄₂ (FIG. 14B) as measured by ELISA in the blood of control (5×FAD) and treated (5×FAD/6KApoEp) mice.

FIGS. 15A-15C show images of representative blots from a Western blot analysis for total Aβ (FIG. 15A), APP β-CFT (FIG. 15B) in control (5×FAD/Ctrl) and treated (5×FAD/6KApoEp). Results were normalized to β-actin (FIG. 15C).

FIGS. 16A-16G show images of representative blots from a Western blot analysis for Alzheimer-like acetylated (a-tau K²⁷⁴ (FIG. 16A) and a-tau K¹⁷⁴ (FIG. 16B)) and phosphorylated tau (p-tau Thr²³¹(FIG. 16D), p-tau Thr⁴⁰⁴ (FIG. 16E), PHF (FIG. 16F), and total tau (FIGS. 16C and 16G) in control (Ctrl) and treated (6KApoEp). Band density ratios of acetylated or phosphorylated tau to total tau was also determined by densitometry analysis.

FIGS. 17A-17B show graphs that demonstrate the results from the densitometry analysis of the Western blots demonstrated in FIGS. 16A-16G. Band density ratios of acetylated or phosphorylated tau to total tau was also determined by densitometry analysis. (Ctrl (control),*P<0.05, **P<0.01, ***P<0.005)

FIGS. 18A-18C show a schematic illustration of an apoE antagonist as described herein (e.g. 6KApoEp) functionally blocking apoE protein or apoE mimic peptide (ApoEp) interaction with N-terminus of APP and inhibiting Aβ generation by reducing APP trafficking from trans-Golgi network and endoplasmic reticulum to the plasma membrane. (FIG. 18A) Under basal conditions, cell surface APP (Cell surf APP) auto internalizes into the cells via clathrin-mediated vesicles but BACE1 internalizes via ADP-ribosylation factor 6 (ARF-6) mediated endocytic pathway. APP β-cleavage occurs when lateral compartmentalization of APP and BACE1 meet in the early endosome at low pH and after additional γ-secretase cleavage generate Aβ, which can be secreted from the cell. In addition, newly synthesized APP travels through the secretory pathway from endoplasmic reticulum (ER) to the plasma membrane (PM) via the trans-Golgi network (TGN). (FIG. 18B) ApoE and/or apoE mimic peptide (ApoEp) interaction with N-terminus of APP increase Aβ generation by promoting internalization of APP via the endocytic pathway. In addition, binding of apoE and/or ApoEp with N-terminal region of APP markedly increases the trafficking of APP from ER and TGN to the plasma membrane, as evidenced by increased cell surface APP and decreased intracellular APP (Intra APP). (FIG. 18C) 6KApoEp inhibits apoE and/or ApoEp physical interaction with N-terminus of APP, which decreases APP endocytosis and trafficking from ER and TGN to the plasma membrane, as evidenced by decreased cell surface and increased intracellular APP, reducing both sAPPa and Aβ generation.

FIGS. 19A-19J can demonstrate that 6KApoEp can markedly inhibit human recombinant and lipidated apoE-mediated Aβ production. CHO/APPwt cells were treated with apoE LDLR binding domain peptide (aa 133-152, ApoEp), FlagApoEp, BiotinApoEp, 3K (lysine) ApoEp, 3D (aspartate) ApoEp or scrambled peptide at 0 to 25 μM, followed after 2 h by analysis of Aβ levels in conditioned media by ELISA (Aβ ELISA, FIGS. 19A and 19C) and after 16 h by analysis of Aβ in cell lysates by Western blot using 82E1 antibody (Aβ WB, FIG. 19B). In addition, CHO/APPwt cells were treated with PBS (Ctrl) or 3K (3 lysines), 6K, 3KApoEp, 6KApoEp, 7KApoEp, 8KApoEp or 9KApoEp at 10 μM, or 6KApoEp at 0-10 μM, for 15 min, followed by additional treatment in the presence or absence of human recombinant apoE4 (apoE4) at 10 μg/mL and then Aβ ELISA (FIGS. 19D-19F) and WB (FIG. 19G). CHO/APPwt cells, SH-SY5Y neuroblastoma cells, hN2TM human neurons and HCN2 cortical neurons were also treated with apoE4, human plasma derived-HDL (HDL) or human plasma-derived apoE3 (hu plas apoE3) at 10 μg/mL, apoE4 pre-incubated with HDL for 1 h at 37° C. (apoE4/HDL), or human astrocyte-derived media (hu astro med) at a 1:4 dilution in the absence or presence of 6KApoEp or scrambled peptide (S pep#2) at 10 μM, followed by Aβ ELISA (FIGS. 19H-19J). Pre-incubating with HDL has been previously reported to lipidate apoE. Hu astro med was obtained from human astrocytes (CCF-STTG1/ATCC® CRL-1718™) as described previously. ELISA results representative of three independent experiments with each condition duplicated and presented as mean±s.d. of Aβ (μg/mg of total intracellular protein). Asterisk indicates P<0.05 for ApoEp-mediated Aβ production compared with control (0) as determined by t test (FIG. 19A), for ApoEp-, FlagApoEp-, BiotinApoEp- or 3DApoEp-mediated Aβ production, compared with scramble peptide (#1)- or 3KApoEp, as determined by one-way ANOVA (FIG. 19C) and for apoE4-, HDL, apoE4/HDL, hu plas apoE3- or hu astro media-mediated Aβ production in the presence compared with the absence of 6KApoEp as determined by t test (FIGS. 19H-19J).

FIGS. 20A-20I can demonstrate that 6KApoEp physically interact with N-terminus of APP. CHO/APPwt cells were treated with 6KApoEp, ApoEp or 6K at 10 μM, or apoE3 or 4 at 10 μg/mL, for 2 h followed by immunoprecipitation of the conditioned media (Condi media) and cell lysates with mouse monoclonal anti-apoE LDLR binding domain antibody (6H3B5). Total secreted APP (t-sAPP) and full-length APP (f-APP) in total (inputs), immunoprecipitates and immunodepleted media and lysates were determined by WB using 22C11 or anti-C-terminal APP751/70 antibody (pC-APP), respectively (FIG. 20A left panels and FIG. 20B, top panels). Alternatively, conditioned media and cell lysates were IP with anti-N-terminal APP41/66 antibody (pN-APP) and apoE, or 6KApoEp and ApoEp, was determined using anti-human apoE antibody (ApoE1H4; FIG. 20A, right panels) or 6H3B5, respectively (FIG. 20B, bottom panels). In addition, CHO/APPwt cells were plated at 8 well-chambers at 1×105/well for 24 h, treated with 6K, ApoEp or 6KApoEp at 10 μM for 2 h, fixed in 4% paraformaldehyde and stained with 6H3B5 and pN-APP antibodies (FIG. 20C). Alexa Fluor 488 goat anti-mouse IgG (green) was used to detect ApoEp and 6KApoEp, while Alexa Fluor 594 donkey anti-rabbit IgG (red) was used to detect N-terminal APP. DAPI co-staining was done to show nuclear DNA. Homogenates prepared from three TgAPPwt mice (2 female/1 male), two AD patients (AD1/male, AD2/female) and two normal age-matched control cortices (Ctrl1/male, Ctrl2/female) were also IP with 6H3B5 or pN-APP and t-APP and apoE were determined using 6E10 and mApoE, respectively (FIGS. 20D-20E). As in vitro confirmation, human recombinant sAPPβ (rsAPPβ, without Aβ domain), rsAPPα, CHO/APPwt cell conditioned medium-derived sAPPα (cmsAPPα) or APP N-terminal peptide (aa 31-60, APP31/60) at 100 nM was incubated with 6KApoEp or 6K at 100 nM, or apoE3 or apoE4 at 10 μg/mL, at 37° C. for 1 h followed by IP with 6H3B5. sAPPa/P, 6KApoEp, APP31/60 and apoE in total (inputs) and immunoprecipitates were determined using 22C11, 6H3B5, pN-APP and ApoE1H4 antibodies, respectively (FIGS. 20F-20G). Human sAPPα protein at 100 nM was also incubated with apoE4 protein at 100 nM in the absence or presence of 6KApoEp or 6K at 5 or 10 μM for 1 h at 37° C., followed by IP with pN-APP antibody and determination of apoE4 and sAPPα in precipitates using ApoE1H4 and 22C11 antibodies, respectively (FIG. 20H). Band density ratios of apoE4 to total sAPPα was determined by densitometry analysis of three independent experiments with each condition duplicated (FIG. 20I). Overall, 6KApoEp, ApoEp, apoE3 and apoE4, but not 6K, immunoprecipitated and localized with N-terminal APP in vivo and in vitro (FIGS. 20A-20G) and 6KApoEp, but not 6K, reduced co-immunoprecipitation of sAPPα with apoE4 (FIGS. 20H-20I).

Note that ApoE1H4 does not detect 6KApoEp because it does not recognize the LDLR binding domain (FIG. 20A, right panels). AD patient and age-matched control cortices provided by Banner Sun Health Research Institute (Sun City, Ariz., USA). Asterisk indicates P<0.05 for apoE4/sAPPα in the presence of 6KApoEp at 10 μM compared with 6KApoEp at 0 or 5 μM or 6K at 10 μM as determined by t test. The results shown in FIGS. 20A-20H panels are representative of two to three independent experiments.

FIGS. 21A-21D can demonstrate that truncated apoE lacking the LDLR binding domain failed to promote Aβ production in CHO/APPwt cells. Conditioned media collected from CHO/APPwt cells co-expressing human wild-type apoE4 (CHO/APPwt/ApoEwt), truncated apoE4 lacking the LDLR binding domain (CHO/APPwt/ApoEtrun) or control vector (CHO/APPwt/Ctrl) were immunoprecipitated with pN-APP and then apoE and sAPPα in total (inputs) and immunoprecipitates were determined by WB using mApoE and 22C11 antibodies, respectively (FIG. 21A). In addition, CHO/APPwtApoEwt, CHO/APPwt/ApoEtrun and CHO/APPwt/Ctrl cells were cultured on 24 well plates at 1×105/well overnight and then treated with 6KApoEp or ApoEp at 10 μM, and CHO/APPwt/Ctrl cells were treated with media collected from CHO/APPwt/ApoEwt or CHO/APPwt/ApoEtrun cells, for 2 h followed by Aβ ELISA (FIG. 21B). CHO/APPwt/ApoEwt, CHO/APPwt/ApoEtrun or CHO/APPwt/Ctrl cells were also plated at 8 well-chambers at 1×105/well for 24 h, fixed in 4% paraformaldehyde and stained with mouse monoclonal anti-N-terminal APP (22C11) and rabbit monoclonal anti-apoE antibodies (mApoE, FIG. 21C). Alexa Fluor 488 goat anti-mouse IgG (green) was used to detect cell surface APP, while Alexa Fluor 594 donkey anti-rabbit IgG (red) was used to detect apoE. FIG. 21D shows domains in apoE, including the LDLR-binding region. ApoEwt but not ApoEtrun co-immunoprecipitated (FIG. 21A) and co-localized with sAPP (FIG. 21C, left panels merged) and enhanced Aβ production (FIG. 21B), indicating that the LDLR binding domain is necessary for apoE interaction with the N-terminus of APP. Notably, 6KApoEp markedly reduced Aβ production in CHO/APPwt/ApoEwt cells, while ApoEp significantly restored Aβ production in CHO/APPwt/ApoEtrun cells (FIG. 21B). ELISA results are representative of three independent experiments with each condition duplicated and presented as means±s.d. of Aβs (μg/mg total intracellular protein). Asterisks indicates P<0.05 for Aβ production in the presence compared with corresponding absence of 6KApoEp or ApoEp or in the presence of CHO/APPwt/ApoEwt compared with CHO/APPwt/ApoEtrun media as determined by t test.

FIGS. 22A-22C can demonstrate that ApoE-promoted Aβ production was significantly attenuated by specific antibodies against the apoE LDLR binding domain or the N-terminus of APP or by expression of N-terminally truncated APP. CHO/APPwt cells were treated with apoE3 or 4 protein at 10 μg/mL or ApoEp at 5 μM in the absence or presence of anti-N-terminal APP (22C11) or anti-apoE LDLR binding domain antibody (6H3B5) at 10 μg/mL, or 6KApoEp at 5 μM, for 2 h followed by Aβ ELISA (FIG. 22A). In addition, CHO/APPwt cells were treated with apoE3 or 4 at 2.5 μg/mL or ApoEp at 10 μM in the absence or presence of 22C11 or 6H3B5 at 5, 10 or 20 μg/ml or 6KApoEp at 5, 10 or 20 μM for 2 h followed by Aβ ELISAs (FIG. 22B). CHO cells were also transfected with pCMV6 APP695 or pCMV6 E1 depleted APP695, yielding cells expressing wild type APP695 (CHO/APPwt) or truncated APP695 lacking the N-terminal E1 region (CHO/APPdE1). After 24 h, the cells were treated with 6KApoEp or ApoEp at 10 μM, or apoE3 or apoE4 at 10 μg/mL, for 4 h followed by Aβ ELISA (FIG. 22C). Diagram indicates important domains identified in APP, including N-terminal E1 region (top panel), and APPwt and APPdE1 proteins in cell lysates and conditioned media derived from the transfected CHO cells as determined by WB using 6E10 (middle panels). ApoE3- and 4- and ApoEp-mediated Aβ production was reduced by 22C11, 6H3B5 and 6KApoEp in a dose-dependent fashion, as well as by truncation of the N-terminal region of APP, indicating that apoE-mediated Aβ production is mediated by interaction of apoE LDLR binding domain with N-terminal region of APP. Isotype-matched control IgG failed to reduce apoE3, 4 or ApoEp-mediated Aβ production (data not shown). ELISA results are representative of three independent experiments with each condition triplicated and presented as mean±s.d. of Aβs (μg/mg of total intracellular protein). Asterisk indicates P<0.05 compared with the corresponding absence of 22C11, 6H3B5 or 6KApoEp, or with corresponding CHO/APPwt cells, as determined by one-way ANOVA.

FIGS. 23A-23H can demonstrate that 6KApoEp can inhibit APP trafficking to the cell surface and can mitigate p44/42 MAPK phosphorylation induced by apoE. CHO/APPwt cells were treated with PBS control (Ctrl), 6KApoEp at 10 μM, apoE4 at 10 μg/mL, or 6KApoEp and apoE4, for 2 h followed by Aβ and sAPPα ELISA (FIGS. 23A-23B, top panels) and WB using 82E1 or 2B3 antibody, respectively (FIGS. 23A-23B, lower panels). In addition, full-length APP (f-APP, short and long exp, exposure), α/β-CTF and total APP in cell lysates were analyzed by WB using anti-C-terminal APP750/70 (pC-APP, left panel, FIG. 23C) and anti-N-terminal APP antibody (6E10, right panel, FIG. 23C). Cell lysates were also biotinylated and immunoprecipitated using Neutravidin beads and intracellular proteins obtained by Neutravidin depletion (intra) and cell surface proteins obtained by Neutravidin precipitation (cell surf) were analyzed for f-APP, LDLR and LRP1 by WB using 6E10, anti-LDLR and anti-LRP1 (FIG. 23D). Band density ratios of cell surf or intra to total f-APP was determined by densitometry analysis (FIG. 23E). Cellular membrane associated full-length APP was analyzed by flow cytometry and presented as means±s.d. median FL-1-A (FIG. 23F). Cultured cells were observed by confocal microscopy using anti-N-terminal (22C11) and pC-APP primary antibodies after permeabilization (perm, 0.05% Triton X-100, 5 min) or directly (without perm) (FIG. 23G). CHO/APPwt cells were treated with 6KApoEp or ApoEp at 10 μM, or apoE3 or 4 at 10 μg/mL, in the absence or presence or 6KApoEp at 10 μM, for 0 to 180 min followed by determination of total p44/42 (t-p44/42), t-p38 and phospho-p44/p42 (pp44/42/) and pp38 MAPK levels in cell lysates by WB (FIG. 23H). ELISA results are representative of three independent experiments with each condition triplicated and presented as mean±s.d. Asterisk indicates P<0.05 compared with Ctrl as determined by t test.

FIGS. 24A-24J can demonstrate that peripheral administration of ApoEp significantly enhances amyloid and tau associated pathology. (FIGS. 24A-24B) 3×Tg-AD mice (n=6 mice, 3 female/3 male) at 9 months of age received ApoEp at 250 μg/kg or vehicle control (Ctrl, PBS alone) i.p. daily for 12 weeks. Following sacrifice, Aβ in brain coronal sections were analyzed by immunohistochemistry staining (IHC) using 4G8 antibody (FIGS. 24A-24B). Percentage of Aβ immunoreactive plaques from hippocampus and cortex was quantified by densitometry analysis (FIG. 24C). In addition, Aβ1-40 and Aβ1-42 in brain homogenates were analyzed by ELISA (FIG. 24D), soluble Aβ was determined by WB using 82E1 and total APP (t-APP) and β-CTF were determined using 6E10 antibodies (FIG. 24E). Acetylated tau (ac-tau, K174 and K274) and phosphorylated tau (p-tau, Thr231 and Thr181) were determined by IHC (FIG. 24F) and WB (FIGS. 24G and 24I). Band density ratios of acetylated or phosphorylated tau to total tau was determined by densitometry analysis (FIGS. 24H and 24J). Asterisk indicates P<0.05 compared with vehicle control. ELISA results and band density ratios represented as mean±s.d.

FIGS. 25A-25N can demonstrate that peripheral administration of 6KApoEp markedly reduces β-amyloid and tau associated pathology. 5×FAD mice (n=10 mice, 5 female/5 male) at 6 weeks of age received 6KApoEp at 250 μg/kg or vehicle control (Ctrl, PBS alone) i.p. daily for 12 weeks followed by analysis of Aβ in brain tissue coronal sections by IHC using 4G8 (FIGS. 25A-25B). Percentage of Aβ immunoreactive plaques from hippocampus and cortex was quantified by desitometry analysis (FIG. 25C). In addition, Aβ1-40,-42 in blood plasma and brain homogenates were analyzed by ELISA (FIG. 25D), total APP (t-APP) and β-CTF were determined in brain homogenates by WB using 6E10 antibody and soluble Aβ was determined using 82E1 (FIG. 25E). Phosphorlated tau (p-tau, Thr231 and Thr404) and acetylated tau (ac-tau, K174 and K274) were also determined by IHC (FIGS. 25F and 25G) and WB (FIGS. 25H and 25J) and band density ratios of acetylated or phosphorylated tau to total tau was determined by densitometry analysis (FIGS. 25H and 25K). Neuronal (β-tubulin III, NeuN, synapsin I), pre-synaptic snynaptophysin (synapto), post-synaptic PDS95 and apoptotic cleaved caspase 3 were determined in brain tissue coronal sections by immunofluorescence staining (FIG. 25L) and in brain homogenates by WB (FIGS. 25M and 25N, upper panels). Band density ratios of each protein to β-actin was determined by densitometry analysis (FIGS. 25M and 25N, lower panes). ELISA results and band density ratios represented as mean±s.d. Asterisk indicates P<0.05 versus Ctrl.

FIGS. 26A-26I can demonstrate that 6KApoEp treatment improves hippocampal-dependent learning and memory in 5×FAD mice. Cognitive function and anxiety were determined after treatment of 5×FAD and non-transgenic control mice (n=10 mice, 5 female/5 male) with 6KApoEp or vehicle PBS (Ctrl). Spontaneous alternation, alternate arm returns and total arm entries were determined for Y-maze (FIGS. 26A-26C). Total freezing times during training and contextual and cued testing after training were determined for fear condition testing (FIGS. 26D-26F). In addition, total times exploring familiar and novel objects were determined for novel object recognition testing (FIG. 26G) and total times exploring central and peripheral zones were determined for open field testing (FIGS. 26H and 26I). 5×FAD mice exhibited impaired cognitive functioning as shown by reduced spontaneous alternation, freezing in context and cued test and ability to discriminate between novel and familiar objects compared with Non-Tg mice, which was reversed by 6KApoEp treatment. 5×FAD mice or treatment with 6KApoEp did not exhibit altered anxiety as determined by time spent in center or perimeter of open field test. Data are presented as mean±s.e.m. Asterisk Indicates P<0.05 as determined by ANOVA.

FIGS. 27A-27B can demonstrate that 6KApoEp markedly inhibits cell surface APP trafficking. CHO/APPwt cells were treated with 6KApoEp (5 μM) or ApoE4 (10 μg/mL) in the absence or presence of 6KApoEp, under pre-treatment, co-treatment or pre-incubation conditions, for 2 h followed by analysis of Aβ levels in conditioned media by ELISA (FIG. 27A). In addition, some of the cells were biotinylated, cell lysates were immunoprecipitated using Neutravidin beads and cell surface (cell surf) proteins obtained by Neutravidin precipitation, intracellular (intra) proteins obtained by Neutravidin depletion and total proteins were analyzed for full-length APP by WB using pC-APP (pAPP751/770) (FIG. 27B). A t test revealed a significant difference between ApoE4 and 6KApoEp, co-treatment of 6KApoEp/apoE4, pre-treatment of 6KApoEp/apoE4 or pre-incubation of 6KApoEp/apoE4 (*P<0.05) (FIG. 27A).

FIGS. 28A-28F ApoE4 and 6KApoEp have a more profound effect on Aβ generation in CHO/APPwt cells than in CHO/APPswe cells. CHO/APPwt and CHO/APPswe cells were treated with apoE4 (0, 5 and 10 μg/mL) or 6KApoEp (0, 5 and 10 μM) for 2 h in serum-free medium. Immediately following the treatment, cell supernatants and cell lysates were analyzed by Aβ ELISA (FIGS. 28A-28B) and WB analysis for holo APP and alp-CTF (FIGS. 28C-28D; 6E10, top panels; pC-APP, bottom panels). The Aβ results are represented as mean±s.d. of Aβ1-40, 42 (μg/mg of total intracellular protein) from three independent experiments. Band density ratios (mean±s.d.) of β-CTF to full-length APP was determined by densitometry analysis (FIGS. 28E-28F). The Aβ results are represented as mean±s.d. of Aβ1-40, 42 (μg/mg of total protein) from three independent experiments. The percentages increase or decrease represent the amount of Aβ changed compared to control treatment.

FIGS. 29A-29C can demonstrate that LDLR recycling is not dependent on APP amyloidogenic processing. CHO/APPwt cells were treated with ApoE4 (10 μg/mL), in the absence or presence of β-secretase inhibitor (400 nM) or γ-secretase inhibitor (200 nM), or PBS (Ctrl) in serum-free medium for 2 h followed by analysis of cell supernatants by Aβ1-40, 42 ELISA to confirm Aβ production by ApoE4 and inhibition by β-inhibitor and γ-inhibitor (data not shown). Total APP and β-CTF in cell lysates were analyzed by WB using pC-APP (top panel) and 6E10 antibodies (lower panel, FIG. 29A). The cell lysates were also incubated with anti-LDLR antibody at 4° C. overnight and then LDLR was immunoprecipitated with anti-rabbit magnetic beads, followed by WB analysis of APP and LDLR in immunoprecipitates using pC-APP (top panel) and anti-LDLR antibodies (lowerpanel, FIG. 29B). Some of the remaining cells were biotinylated, lysed and immunoprecipitated using Neutravidin beads. The intracellular (intra) proteins obtained by Neutravidin depletion, cell surface (cell surf) proteins obtained by Neutravidin precipitation and total proteins were subjected to WB analysis using anti-LDLR and anti-A-actin antibodies (FIG. 29C). These results indicate that cell surface expression of LDLR, and by extension LDLR recycling, is not dependent on APP amyloidogenic processing.

FIGS. 30A-30B can demonstrate that 6KApoEp treatment does not alter LDLR and APP mRNA levels. CHO/APPwt cells were treated with 6KApoEp at 10 μM for 2 or 24 h, followed by analysis of LDLR (30A) and APP (30B) mRNA levels using one step real-time PCR kit with SYBR Green. The results are represented as mean±s.d. of threshold cycle (Ct) ratio of APP/β-actin or LDLR/β-actin from two independent experiments.

FIGS. 31A-31C can demonstrate that 6KApoEp treatment does not alter cholesterol uptake or LDLR and apoE expression. CHO/APPwt cells were treated with ApoE3 at 0, 5 and 10 μg/mL or 6KApoEp at 0, 5 and 10 μM overnight followed by analysis of total cell associated cholesterol levels using the flourometric cholesterol quantitation kit (FIG. 31A). Total cell associated cholesterol levels are represented as mean±s.d. (μg/mg of total intracellular protein) from three independent experiments. In addition, total plasma cholesterol levels in 3×Tg-AD mice treated with ApoEp (n=6) and 5×FAD mice treated with 6KApoEp (n=10) or PBS (Ctrl, n=10), as described in FIGS. 25A-26G, and in associated wild-type mice (WT) were analyzed by flourometric cholesterol quantitation kit (FIG. 31B). LDLR (FIG. 31C, top panel) and ApoE (middle panel) expression levels in brain homogenates were also analyzed by WB. These results indicate that brain cholesterol, LDLR and ApoE expression levels in 3×Tg-AD and 5×FAD mice were not altered by ApoEp or 6KApoEp treatment. (*P<0.05) and P>0.0.5, non-significant compared to control treatment.

FIGS. 32A-32C can demonstrate that 6KApoEp treatment significantly inhibits basal and LPS-induced microglial TNFα production by reducing p44/42 MAPK phosphorylation. Murine microglial BV2 cells were treated with 6KApoEp at 0, 2.5, 5 or 10 μM or PBS (Ctrl) in the absence or presence of LPS (100 ng/mL) followed by determination of TNFα in cell supernatants and cell lysates after 3 h by ELISA (FIG. 32A) and of total and phospho-p44/42 MAPK in cell lysates after 30 min by WB (FIG. 32B). In addition, splenocytes were isolated from 5×FAD mice (n=5, 3 F/2 F) treated with 6KApoEp or 6K (300 μg/kg/day) or PBS (Ctrl) i.p. daily for 4 weeks, followed by treatment with anti-CD3 antibody at 5 μg/mL overnight and determination of IFNγ in conditioned media by ELISA (FIG. 32C). ELISA results representative of triplicates and presented as means±s.d. of IFNγ (ng/mg total intracellular protein).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, cell biology, and the like, which are within the skill of the art. Such techniques are explained fully in the literature.

### Definitions

As used herein, “control” is an alternative subject or sample used in an experiment for comparison purpose and included to minimize or distinguish the effect of variables other than an independent variable.

As used herein, “biocompatible” or “biocompatibility” refers to the ability of a material to be used by a patient without eliciting an adverse or otherwise inappropriate host response in the patient to the material or a derivative thereof, such as a metabolite, as compared to the host response in a normal or control patient. As used herein, “biodegradable” refers to the ability of a material or compound to be decomposed by bacteria or other living organisms or organic processes.

As used herein, “about,” “approximately,” and the like, when used in connection with a numerical variable, generally refers to the value of the variable and to all values of the variable that are within the experimental error (e.g., within the 95% confidence interval for the mean) or within ±10% of the indicated value, whichever is greater.

As used herein, “effective amount” is an amount sufficient to effect beneficial or desired results. An effective amount can be administered in one or more administrations, applications, or dosages.

As used herein, “administering” can refer to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “preventative” refers to hindering or stopping a disease or condition before it occurs or while the disease or condition is still in the sub-clinical phase.

As used herein, “therapeutic” can refer to treating or curing a disease or condition.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term farm animal includes a horse, sheep, goat, chicken, pig, cow, donkey, llama, alpaca, turkey, and the like.

The terms “operatively linked” or “operatively coupled” as used herein can refer to the association of nucleic acid sequences on a single nucleic acid fragment so that the function of one is regulated by the other. For example, a promoter is operatively linked with a coding sequence when it is capable of regulating the expression of that coding sequence (i.e., the coding sequence is under the transcriptional control of the promoter). Coding sequences can be operatively linked to regulatory sequences in a sense or antisense orientation. In one example, the complementary RNA regions can be operatively linked, either directly or indirectly, 5′ to the target mRNA, or 3′ to the target mRNA, or within the target mRNA, or a first complementary region is 5′ and its complement is 3′ to the target mRNA. The term “operatively linked” as used herein can also refer to the direct or indirect linkage of any two nucleic acid sequences on a singly nucleic acid fragment such that they are indirectly or directly physically connected on the same nucleic acid fragment. The term “operatively linked” as used herein can also refer to the insertion of a nucleic acid within the 5′ and 3′ end of another nucleic or the direct coupling of a nucleic acid to the 5′ or 3′ end of another nucleic acid.

As used herein, “specific binding,” “specifically bind,” and the like refer to binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins. As another non-limiting example, a miRNA can specifically bind preferably to a miRNA target and not to a non-specific nucleic acid sequence or if binding to a non-specific nucleic acid sequence occurs that no change in the expression or function of the non-specific nucleic acid can be observed or detected.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “polypeptides” or “proteins” are amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lie, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “gene” can refer to a hereditary unit corresponding to a sequence of DNA that occupies a specific location on a chromosome and that contains the genetic instruction for a characteristic(s) or trait(s) in an organism. “Gene” also refers to the specific sequence of DNA that is transcribed into an RNA transcript that can be translated into a polypeptide or be a catalytic RNA molecule including but not limited to tRNA, siRNA, piRNA, miRNA, long-non-coding RNA and shRNA.

As used herein, “deoxyribonucleic acid (DNA)” and “ribonucleic acid (RNA)” generally refer to any polyribonucleotide or polydeoxribonucleotide, which can be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers or coding mRNA (messenger RNA).

As used herein, “nucleic acid sequence” and “oligonucleotide” also encompasses a nucleic acid and polynucleotide as defined elsewhere herein.

As used herein, “DNA molecule” includes nucleic acids/polynucleotides that are made of DNA.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, polynucleotide as used herein refers to triple-stranded regions comprising RNA or DNA or both RNA and DNA. The strands in such regions can be from the same molecule or from different molecules. The regions can include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotide” as that term is intended herein.

As used herein, “microRNA” can refer to a small non-coding RNA molecule containing about 21 to about 23 nucleotides found in organisms, which functions in transcriptional and post-transcriptional regulation of transcription and translation of RNA. “MicroRNA” can exist as part of a larger nucleic acid molecule such as a stem-loop structure that can be processed by a cell and yield a microRNA of about 21-23 nucleotides.

As used herein, “pharmaceutically acceptable carrier, diluent, binders, lubricants, glidants, preservative, flavoring agent, coloring agent, and excipient” refers to a carrier, diluent, binder, lubricant, glidant, preservative, flavoring agent, coloring agent, or excipient that is useful in preparing a pharmaceutical formulation that is generally safe, non-toxic, and is neither biologically or otherwise undesirable, and includes a carrier or excipient that is acceptable for veterinary use as well as human pharmaceutical use.

The term “treating”, as used herein, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “overexpressed” or “overexpression” refers to an increased expression level of an RNA (coding or non-coding RNA) or protein product encoded by a gene as compared to the level of expression of the RNA or protein product in a normal or control cell.

As used herein, “underexpressed” or “underexpression” refers to decreased expression level of an RNA (coding or non-coding RNA) or protein product encoded by a gene as compared to the level of expression of the RNA or protein product in a normal or control cell.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein with reference to the relationship between DNA, cDNA, cRNA, RNA, and protein/peptides, “corresponding to” or “encoding” can refer to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “promoter” can include all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, “selectable marker” or can refer to a gene whose expression allows one to identify cells that have been transformed with a nucleic acid (naked, contained in a vector, or a plasmid) containing the selectable marker gene. For instance, a recombinant nucleic acid may include a selectable marker operatively linked to a gene of interest (e.g. a gene encoding a recombinant polypeptide) and a promoter, such that expression of the selectable marker indicates the successful transformation of the cell with the gene of interest. In some instances the selectable marker gene can be operative linked to a gene of interest such that when they are expressed as proteins the selectable marker allows for identification of expression of the gene of interest. “Selectable marker” can also be referred to herein as a reporter gene or polypeptide.

As used herein, “constitutive promoter” can refer to a promoter that allows for continual or ubiquitous transcription of its associated gene or polynucleotide. Constitutive promoters are generally are unregulated by cell or tissue type, time, or environment.

As used herein, “inducible promoter” can refer to a promoter that allows transcription of its associated gene or polynucleotide in response to a substance or compound (e.g. an antibiotic, or metal), an environmental condition (e.g. temperature), developmental stage, or tissue type.

As used herein, “electroporation” is a transformation method in which a high concentration of plasmid DNA (containing exogenous DNA) or RNA is added to a suspension of host cell protoplasts, and the mixture shocked with an electrical field of about 200 to 600 V/cm.

As used herein, “plasmid” can refer to a non-chromosomal double-stranded DNA sequence including an intact “replicon” such that the plasmid is replicated in a host cell.

As used herein, the term “vector” can refer to a vehicle used to introduce an exogenous nucleic acid sequence into a cell. A vector can include a DNA molecule, linear or circular (e.g. plasmids), which includes a segment encoding a polypeptide of interest operatively linked to additional segments that provide for its transcription and translation upon introduction into a host cell or host cell organelles. Such additional segments can include promoter and terminator sequences, internal ribosome entry site, and may also include one or more origins of replication, one or more selectable markers, an enhancer, a polyadenylation signal, microRNA target sequences etc. Expression vectors are generally derived from yeast or bacterial genomic or plasmid DNA, or viral DNA, or can contain elements of both. The term “vector” can also include RNA or circular RNA vectors linked to additional segments that provide for its translation upon introduction into a host cell or host cell organelles. Such additional segments can include 5′Cap, one or more selectable markers, an enhancer, a polyadenylation signal, polyA tail, microRNA target sequences etc. The term vector includes viral vectors and multi-vector viral vector systems (adeno, lentiviral, retroviral, and the like) that can be used to introduce a transgene into the genome of a cell. Such viral vectors and viral vector systems will be generally known by those skilled in the art.

As used herein, “identity,” can refer to a relationship between two or more polypeptide sequences, as determined by comparing the sequences. In the art, “identity” also refers to the degree of sequence relatedness between polypeptide as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W, Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA (unmodified or modified), it may be associated with various proteins or regulatory elements (e.g., a promoter and/or signal element, miRNA target sequences as described herein), or the nucleic acid may be incorporated into a vector or a chromosome.

As used herein, “transformation” or “transformed” refers to the introduction of a nucleic acid (e.g., DNA or RNA) into cells in such a way as to allow expression of the coding or non-coding portions of the introduced nucleic acid.

As used herein a “transformed cell” is a cell transformed with a nucleic acid sequence.

As used herein, a “transgene” refers to an artificial gene which is used to transform a cell of an organism, such as a bacterium or a plant.

As used herein, “transgenic” refers to a cell, tissue, or organism that contains a transgene.

As used herein, the term “recombinant” generally refers to a non-naturally occurring nucleic acid, nucleic acid construct, or polypeptide. Such non-naturally occurring nucleic acids can include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a “fusion protein” (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g, a nucleic acid and a constitutive promoter etc.). Recombinant also refers to the polypeptide encoded by the recombinant nucleic acid. Non-naturally occurring nucleic acids or polypeptides include nucleic acids and polypeptides modified by man, including but not limited to miRNA target sequences described herein.

As used herein, the term “exogenous DNA” or “exogenous RNA” or exogenous nucleic acid sequence” or “exogenous polynucleotide” refers to a nucleic acid sequence that was introduced into a cell, organism, or organelle via transfection or transduction. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

### Discussion

Alzheimer's disease (AD) is an irreversible, progressive brain disorder that slowly destroys memory and thinking skills, and eventually the ability to carry out the simplest of tasks. AD is currently the sixth leading cause of death in the United States and ranks third in as the leading cause of death in the elderly. Part of the pathology of AD is the abnormal formation of amyloid plaques and tau tangles in the brain and loss of connections between neurons. Amyloid plaques are made up of amyloid beta peptides (Aβ), which are fragments of an amyloid precursor protein (APP).

Apolipoprotein E (ApoE) is a class of apolipoprotein found in the chylomicron and Intermediate-density lipoprotein (IDL) that is essential for the normal catabolism of triglyceride-rich lipoprotein constituents (Liu et al., 2013). In peripheral tissues, ApoE is primarily produced by the liver and macrophages, and mediates cholesterol metabolism in an isoform-dependent manner. In the central nervous system, ApoE is mainly produced by astrocytes, and transports cholesterol to neurons via APOE receptors, including LRP, which are members of the low density lipoprotein receptor (LDLR) gene family (Liu et al., 2013). Importantly, ApoE is involved in Alzheimer's disease and cardiovascular disease (lan P. Stolerman, 2010).

ApoE is 299 amino acids long and contains multiple amphipathic α-helices. According to crystallography studies, a hinge region connects the N- and C-terminal regions of the protein. The N-terminal region (residues 1-167) forms an anti-parallel four-helix bundle such that the non-polar sides face inside the protein. Meanwhile, the C-terminal domain (residues 206-299) contains three α-helices which form a large exposed hydrophobic surface and interact with those in the N-terminal helix bundle domain through hydrogen bonds and salt-bridges. The N-terminal region also contains the low density lipoprotein receptor (LDLR)-binding site (residues 134-150) (Philips et al., 2014).

ApoE transports lipoproteins, fat-soluble vitamins, and cholesterol into the lymph system and then into the blood. It is synthesized principally in the liver, but has also been found in other tissues such as the brain, kidneys, and spleen (Baars et al., 2011). In the nervous system, non-neuronal cell types, most notably astroglia and microglia, are the primary producers of ApoE, while neurons preferentially express the receptors for ApoE (Zhang et al., 2013). There are seven currently identified mammalian receptors for ApoE, including LRP, which belong to the evolutionarily conserved LDLR family (Rogers et al., 2008).

APOE is polymorphic (Singh et al., 2006; Eisenberg et al., 2010) with three major alleles: ApoE2 (cys112, cys158), ApoE3 (cys112, arg158), and ApoE4 (arg112, arg158) (Baars et al., 2011; Ghebranious et al., 2005). Although these allelic forms differ from each other by only one or two amino acids at positions 112 and 158, these differences alter ApoE structure and function, which have physiological consequences.

ApoE2 (rs7412), which has an allele frequency of approximately 7 percent, binds poorly to cell surface receptors compared with ApoE3 and E4 (Giveira et al., 1996). Most interestingly, ApoE2, which is severely defective in LDLR binding activity, with 2% LDLR binding activity compared with ApoE3, differs structurally from ApoE3 and ApoE4, which bind avidly to LDLR. This reduced binding affinity of ApoE2 is likely mediated by the presence of cysteine rather than arginine in position 158, thereby altering the conformation and size of the positive potential in the binding region (Mahley et al., 2009). ApoE3 (rs429358), which has an allele frequency of approximately 79 percent (Alzheimer Research Forum), is considered the “neutral” Apo E genotype. In this isoform, the 134-150 binding region is largely solvent exposed and forms a 20 Å field of positive potential, likely available for receptor binding. ApoE4, which has an allele frequency of approximately 14 percent, has been implicated in atherosclerosis, Alzheimer's disease and impaired cognitive function (Corder et al., 1993; Deary et al., 2002.

APP can also act as a receptor for several ligands, based on its structural and functional similarities with other type I transmembrane receptors and γ-secretase substrates, including Notch and Deleted in the colorectal carcinoma (DCC)²⁵⁻²⁷ Notably, after ligand binding to these receptors its proteolytic products mediate intracellular signaling and transcription involved in neuronal outgrowth, synaptogenesis and axon guidance. APP in particular has two extracellular heparin binding domains which bind negatively charged molecules such as heparin and matrix proteins, one of high affinity named E1 or growth factor-like domain (GFLD) located at the N-terminus of APP and another of lower affinity named E2 located proximal to the membrane. Disulfide bridges between adjacent APP molecules at the E1 domain resembles other receptor-like proteins, appears to stabilize the APP structure at the cell surface and favors ligand induction of signaling cascade^{28,29}. In addition, the E2 domain can bind albeit with low affinity to membrane-anchored heparan sulfate proteoglycans (HSPGs), which can act as a co-receptor and allow putative ligands to bind with higher affinity ³⁰⁻³¹. However, unlike the case of Notch and DCC, the putative ligand(s) of APP has never been characterized.

Early studies utilizing yeast two-hybrid and immunoprecipitation of cell culture supernatants suggested that the N-terminus of APP can directly bind apoE³². The domain where interaction between APP and apoE occurred in these experiments was reported to be the amino-terminal region of APP, between amino acids 1-207 and upstream of the Aβ region, and the amino-terminal region of apoE, between amino acids 1-191. In addition, apoE enhanced intracellular APP endocytosis and/or retention and reduced sAPPα production. More recent studies indicate that both glia-derived and recombinant apoE stimulates Aβ production in embryonic stem cell-derived human neurons with a rank order of potency of apoE4>apoE3>apoE2, mediated by a signal transduction cascade involving activation of a non-canonical mitogen associated protein kinase (MAPK) p44/p42 and APP transcription/translation ³³. Altogether these studies suggest that apoE can bind to the N-terminal region of APP, thereby enhancing APP endocytosis and directing its processing from sAPPα to Aβ production.

With that said, described herein are compounds, compositions, and formulations that can modulate the binding of an ApoE protein to an LDL receptor, an ApoE receptor, and/or APP. Also described herein are recombinant polypeptides that can contain an ApoE LDLR binding domain, and in some embodiments, additional lysine residues. The compounds, compositions, and formulations described herein can reduce ApoE protein binding to an LDLR, ApoE receptor, and/or APP. Also described herein are polynucleotides configured to encode the recombinant polynucleotides. Also described herein are cells and transgenic animals that can contain one or more recombinant polypeptides or polynucleotides described herein. Also provided herein are methods of administering the compounds, compositions, and formulations described herein to a subject. The subject can be suffering from a cardiovascular, vascular, or neurologic disease or disorder. The subject can be suffering from AD. The compounds, compositions, and formulations can be useful to treat cardiovascular, vascular, and/or neurologic diseases. Also provided herein are assays for detecting compounds, compositions, and formulations capable of modulating the binding of an ApoE protein to an LDL receptor, an ApoE receptor, and/or APP.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Compositions

Provided herein are compositions configured to modulate binding of a protein to an ApoE receptor. ApoE receptors are known in the art an include LDL receptor (LDLR), Very low-density lipoprotein receptors (VLDLRs), Apoer2, and lipoprotein receptor-related protein 1 (LRP1). The LDL receptor is generally known in the art and will be instantly appreciated by those of skill in the art. See e.g. Brown and Goldstein (1979) Proc. Natl. Acad. Sci. 76(7):3330-3337; Hobbs et al., (1993) Hum. Mutat. 1(6): 445-466; May et al., (2003) Sci. STKE (175): PE12; and Braakman (2004) Cell. Mol. Life Sci. 61(19-20): 2461-2470. The compositions can be configured to reduce or decrease the amount of binding of an ApoE protein to an ApoE receptor as compared to a suitable control. Suitable controls will be instantly appreciated by one of ordinary skill in the art.

In some embodiments, the composition can include a compound that can specifically have the same sequence as the LDLR binding domain of the ApoE protein. In some embodiments, the LDLR binding domain of the ApoE protein can have a sequence according to SEQ ID NO.: 1. In other embodiments, the LDLR binding domain of the ApoE protein can have a sequence that is about 90% to about 100% identical to SEQ ID NO.: 1. In further embodiments, the compound can specifically have the same sequence as a fragment of the LDLR binding domain wherein the fragment can contain at least 5 contiguous amino acids of SEQ ID NO:.1 (LRVRLASHLRKLRKRLLRDA) or a sequence that is about 90% to about 100% identical to SEQ ID NO.: 1

In some embodiments, the compound that can modulate binding of an ApoE protein and an ApoE receptor can be an antibody or fragment thereof. The antibody can be configured to specifically bind to a LDLR binding domain of the ApoE protein. In embodiments, the LDLR binding domain of the ApoE protein can have a sequence according to SEQ ID NO.: 1. In other embodiments, the LDLR binding domain of the ApoE protein can have a sequence that is about 90% to about 100% identical to SEQ ID NO.: 1. In further embodiments, the antibody or fragment thereof can specifically bind to a fragment of the LDLR binding domain wherein the fragment can contain at least 5 contiguous amino acids of SEQ ID NO: 1 (LRVRLASHLRKLRKRLLRDA) or a sequence that is about 90% to about 100% identical to SEQ ID NO.: 1. The antibody can be a monoclonal antibody or a polyclonal antibody. Methods of producing antibodies and screening antibodies are generally known in the art and will be appreciated by one of ordinary skill in the art.

In other embodiments, the compound that can be configured to modulate binding of ApoE protein to an ApoE protein receptor can be a small molecule compound. As used in this context, “small molecule” can refer to a low molecular weight organic or inorganic compound or composition (less than about 900 daltons) that can elicit a biologic response in a cell, tissue, organ, and/or organism and having a size on the order of about 10⁻⁹ m or less.

In some embodiments, the compound can be a competitive inhibitor or antagonist of an ApoE protein for an ApoE receptor, LDLR, and/or APP. In other words, in some embodiments, the compound can compete with or otherwise inhibit ApoE binding to the ApoE receptor, LDLR, and or APP. While not being bound to theory, in this way the compound can reduce binding of the ApoE to the ApoE receptor, LDLR, and/or APP.

In some embodiments, the compound can be a recombinant polypeptide. The recombinant polypeptide can contain a polypeptide sequence according to SEQ ID NO: 1 or a polypeptide sequence that is about 90-100% identical to SEQ ID NO: 1 and at least 3 or at least 6 additional lysine residues operatively coupled to the polypeptide sequence according to SEQ ID NO: 1 or the polypeptide sequence that is about 90-100% identical SEQ ID NO: 1. In some embodiments, the number of additional lysine residues can range from 3 to 20. In other embodiments the number of additional lysine residues can range from 3-9. In some embodiments, the at least 3 lysine or at least 6 additional residues can be operatively coupled to the N-terminus of the polypeptide sequence according to SEQ ID NO: 1 or the polypeptide sequence that is about 90-100% identical SEQ ID NO: 1. In other embodiments, the at least 3 or the at least 6 additional lysine residues are operatively coupled to the C-terminus of the polypeptide sequence according to SEQ ID NO: 1 or the polypeptide sequence that is about 90-100% identical SEQ ID NO: 1. In further embodiments, the at least 3 or the at least 6 additional lysine residues are operatively coupled between the N-terminus and C-terminus of the polypeptide sequence according to SEQ ID NO: 1 or the polypeptide sequence that is about 90-100% identical SEQ ID NO: 1. In some embodiments, the recombinant polypeptide can be according to any one of SEQ ID NOs: 2-9. In some embodiments the number of additional lysine residues can be 6.

Recombinant Polypeptides, Polynucleotides, and Vectors

Provided herein are recombinant polypeptides that can contain a polypeptide sequence according to any one of SEQ ID NOs.: 1-9 or a polypeptide sequence that is about 90-100% identical to any one of SEQ ID NOs.: 1-9. The recombinant polypeptides can include one or more reporter proteins (also referred to as selectable markers) operatively linked to the polypeptide sequence according to any one of SEQ ID NOs.: 1-9 or a polypeptide sequence that is about 90-100% identical to any one of SEQ ID NOs.: 1-9. Exemplary reporter proteins include but are not limited to β-galactosidase, GUS; fluorescent proteins such as green fluorescent protein (GFP), cyan (CFP), yellow (YFP), red (RFP), luciferase, cell surface proteins and, epitope tags such as but not limited to, e.g. FLAG- and His-tags.

Modifications and changes can be made in the structure of the polypeptides of the present disclosure that result in a molecule having similar characteristics as the unmodified polypeptide (e.g., a conservative amino acid substitution). Modification techniques are generally known in the art. For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a functional variant Polypeptides with amino acid sequence substitutes that still retain properties substantially similar to polypeptides corresponding to any one of SEQ ID NOs.: 1-9 or a polypeptide sequence that is about 90-100% identical to any one of SEQ ID NOs.: 1-9 are within the scope of this disclosure.

Also provided herein are polynucleotides that can encode on or more of the recombinant polypeptides described herein. The polynucleotides can further include one or more selectable marker (or reporter) genes. Examples of selectable markers include, but are not limited to, DNA and/or RNA segments that contain restriction enzyme sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; epitope tags (e.g. FLAG- and His-tags), and, the inclusion of a DNA sequences required for a specific modification (e.g., methylation) that allows its identification. Other suitable markers will be appreciated by those of skill in the art.

In some embodiments, non-coding nucleotides can be placed at the 5′ and/or 3′ end of the polynucleotides encoding a polypeptide sequence according to any one of SEQ ID NOs.: 1-9 or a polypeptide sequence that is about 90-100% identical to any one of SEQ ID NOs.: 1-9 without affecting the functional properties of the molecule. A polyadenylation region at the 3′-end of the coding region of a polynucleotide can be included. The polyadenylation region can be derived from the endogenous gene, from a variety of other plant genes, from T-DNA, or through chemical synthesis. In further embodiments, the nucleotides encoding the polypeptide sequence according to any one of SEQ ID NOs.: 1-9 or a polypeptide sequence that is about 90-100% identical to any one of SEQ ID NOs.: 1-9 can be conjugated to a nucleic acid encoding a signal or transit (or leader) sequence at the N-terminal end (for example) of the polypeptide sequence according to any one of SEQ ID NOs.: 1-9 or a polypeptide sequence that is about 90-100% identical to any one of SEQ ID NOs.: 1-9 that co-translationally or post-translationally directs transfer of the polypeptide sequence according to any one of SEQ ID NOs.: 1-9 or a polypeptide sequence that is about 90-100% identical to any one of SEQ ID NOs.: 1-9. The polynucleotide sequence can also be altered so that the polypeptide sequence according to any one of SEQ ID NOs.: 1-9 or a polypeptide sequence that is about 90-100% identical to any one of SEQ ID NOs.: 1-9 is conjugated to a linker, selectable marker, or other sequence for ease of synthesis, purification, and/or identification of the protein. In one embodiment, the recombinant polynucleotide sequence includes at least one regulatory sequence operatively linked to the isolated nucleotide or cDNA sequences or fragments thereof.

To express a recombinant polynucleotide that encodes a polypeptide sequence according to any one of SEQ ID NOs.: 1-9 or a polypeptide sequence that is about 90-100% identical to any one of SEQ ID NOs.: 1-9 in a cell, the recombinant polynucleotide can be combined (e.g., in a vector) with transcriptional and/or translational initiation regulatory sequences, i.e. promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, μF-1α, β-actin, RSV, and PGK. Suitable constitutive promoters for bacterial cells, yeast cells, fungal cells are generally known in the art, such as a T-7 promoter for bacterial expression and an alcohol dehydrogenase promoter for expression in yeast.

In other embodiments, tissue-specific promoters or inducible promoters may be employed to direct expression of the exogenous nucleic acid in a specific cell type, under certain environmental conditions, and/or during a specific state of development. Suitable tissue-specific and inducible promoters are generally known in the art. In some embodiments, the tissue specific promoter is a brain, neuron, and/or neuron support cell specific promoter. For example, the calcium-calmodulin dependent protein kinase Ila (CaM-Klla) promotor can be used, which can be specific for neurons of the forebrain. Other suitable brain, neuron, and neuron support cell specific promoters will be appreciated by those of ordinary skill in the art.

Also provided herein are vectors that can contain one or more of the polynucleotides or described herein. In embodiments, the vector can contain one or more polynucleotides that can encode a polypeptide according to any one of SEQ ID NOs.: 1-9 or a polypeptide sequence that is about 90-100% identical to any one of SEQ ID NOs.: 1-9. The vectors can be useful in producing transgenic bacterial, fungal, yeast, animal cells, and transgenic animals that can express a polypeptide according to any one of SEQ ID NOs.: 1-9 or a polypeptide sequence that is about 90-100% identical to any one of SEQ ID NOs.: 1-9. Within the scope of this disclosure are vectors containing one or more of the polynucleotide sequences described herein.

Cells and Transgenic Animals

Also provided herein are cells that are transformed with one or more polynucleotides (including vectors) described herein. The cells that are transformed with one or more polynucleotides described can express one or more recombinant polypeptides described herein. The cells can be bacterial, yeast, fungi, plant, or mammalian. Techniques for transforming cells are generally known in the art and can include, but are not limited to, transfection, electroporation, gene gun, and viral vector mediated transduction. The cells can be useful in the production of the recombinant polypeptides described herein. The cells can be useful in an assay to screed for candidate compounds that can bind or otherwise interact with and/or modulate the polypeptides described herein.

Also provided herein are transgenic animals, including but not limited to, mice, chickens, and pigs, that express one or more polypeptides described herein. Methods for producing transgenic animals that can express recombinant polypeptides are generally known in the art and will be appreciated by those of skill in the art.

Pharmaceutical Formulations

Also provided herein are pharmaceutical formulations containing an amount of a cell-selective RNA molecule, corresponding DNA molecule (including vectors), and/or viron particle as described herein. The amount can be an effective amount. Pharmaceutical formulations can be formulated for delivery via a variety of routes and can contain a pharmaceutically acceptable carrier. Techniques and formulations generally can be found in Remmington's Pharmaceutical Sciences, Meade Publishing Co., Easton, Pa. (20^{th} Ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxyl methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

The pharmaceutical formulations can be administered to a subject in need thereof. The subject in need thereof can have a disease, disorder, or a symptom thereof. Example disease or disorder can include, but are not limited to, a cardiovascular disease, a pulmonary disease, a brain disease, a renal disease, a liver disease, a blood disease, a nervous system disease, an intestinal disease, an ocular disease, and cancer. The pharmaceutical formulations can be disposed on or otherwise coupled to or integrated with a medical device, such as, but not limited to, catheters or stents, such that the pharmaceutical formulation is eluted from the medical device over a time period. The pharmaceutical formulation can therefore be delivered to a subject in need thereof during and/or after a procedure such as an angioplasty, vein draft or organ transplant. Other procedures where such a medical device would be useful will be appreciated by those of skill in the art.

A pharmaceutical formulation can be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. The construct, biologic molecules and pharmaceutical formulations thereof described herein can be disposed on or otherwise integrated with or coupled to a medical device such as, but not limited to, a catheter or stent, such that the construct, biological molecule can be released to the surrounding local area or systemically over a period of time after insertion or implantation into a subject in need thereof. These can also be referred to as drug eluting medical devices.

Pharmaceutical formulations suitable for injectable use can include sterile aqueous solutions (where water soluble) or dispersions and sterile powders for the extemporaneous preparation of sterile injectable solutions or dispersions. For intravenous administration, suitable carriers can include physiological saline, bacteriostatic water, Cremophor EM™ (BASF, Parsippany, N.J.) or phosphate buffered saline (PBS). Injectable pharmaceutical formulations can be sterile and can be fluid to the extent that easy syringability exists. Injectable pharmaceutical formulations can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of injectable compositions can be brought about by incorporating an agent which delays absorption, for example, aluminum monostearate and gelatin.

Sterile injectable solutions can be prepared by incorporating any of the compositions or recombinant polypeptides as described herein in an amount in an appropriate solvent with one or a combination of ingredients enumerated herein, as required, followed by filtered sterilization. Generally, dispersions can be prepared by incorporating the nucleic acid vectors into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fluidic acid derivatives. Transmucosal administration can be accomplished through the use of nasal sprays or suppositories. For transdermal the compositions or recombinant polypeptides described herein can be formulated into ointments, salves, gels, or creams as generally known in the art. In some embodiments, the compositions or recombinant polypeptides can be applied via transdermal delivery systems, which can slowly release the compositions or recombinant polypeptides for percutaneous absorption. Permeation enhancers can be used to facilitate transdermal penetration of the active factors in the conditioned media. Transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475.

Administration of the compositions or recombinant polypeptides described herein is not restricted to a single route, but may encompass administration by multiple routes. For instance, exemplary administrations by multiple routes include, among others, a combination of intradermal and intramuscular administration, or intradermal and subcutaneous administration. Multiple administrations may be sequential or concurrent. Other modes of application by multiple routes will be apparent to the skilled artisan.

The pharmaceutical formulations can be administered to a subject by any suitable method that allows the agent to exert its effect on the subject in vivo. For example, the formulations or other compositions described herein can be administered to the subject by known procedures including, but not limited to, by oral administration, sublingual or buccal administration, parenteral administration, transdermal administration, via inhalation, via nasal delivery, vaginally, rectally, and intramuscularly. The formulations or other compositions described herein can be administered parenterally, by epifascial, intracapsular, intracutaneous, subcutaneous, intradermal, intrathecal, intramuscular, intraperitoneal, intrasternal, intravascular, intravenous, parenchymatous, and/or sublingual delivery. Delivery can be by injection, infusion, catheter delivery, or some other means, such as by tablet or spray.

For oral administration, a formulation as described herein can be presented as capsules, tablets, powders, granules, or as a suspension or solution. The formulation can contain conventional additives, such as lactose, mannitol, cornstarch or potato starch, binders, crystalline cellulose, cellulose derivatives, acacia, cornstarch, gelatins, disintegrators, potato starch, sodium carboxymethylcellulose, dibasic calcium phosphate, anhydrous or sodium starch glycolate, lubricants, and/or or magnesium stearate.

For parenteral administration (i.e., administration by through a route other than the alimentary canal), the formulations described herein can be combined with a sterile aqueous solution that is isotonic with the blood of the subject. Such a formulation can be prepared by dissolving the active ingredient in water containing physiologically-compatible substances, such as sodium chloride, glycine and the like, and having a buffered pH compatible with physiological conditions, so as to produce an aqueous solution, then rendering the solution sterile. The formulation can be presented in unit or multi-dose containers, such as sealed ampoules or vials.

The formulation can be delivered by injection, infusion, or other means known in the art.

For transdermal administration, the formulation described herein can be combined with skin penetration enhancers, such as propylene glycol, polyethylene glycol, isopropanol, ethanol, oleic acid, N-methylpyrrolidone and the like, which increase the permeability of the skin to the nucleic acid vectors of the invention and permit the nucleic acid vectors to penetrate through the skin and into the bloodstream. The formulations and/or compositions described herein can be further combined with a polymeric substance, such as ethylcellulose, hydroxypropyl cellulose, ethylene/vinyl acetate, polyvinyl pyrrolidone, and the like, to provide the composition in gel form, which can be dissolved in a solvent, such as methylene chloride, evaporated to the desired viscosity and then applied to backing material to provide a patch.

Dosage Forms

The pharmaceutical formulations or compositions described herein can be provided in unit dose form such as a tablet, capsule or single-dose injection or infusion vial. Where appropriate, the dosage forms described herein can be microencapsulated. The dosage form can also be prepared to prolong or sustain the release of any ingredient. In some embodiments, the complexed active agent can be the ingredient whose release is delayed. In other embodiments, the release of an auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Coatings may be formed with a different ratio of water soluble polymer, water insoluble polymers, and/or pH dependent polymers, with or without water insoluble/water soluble non polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

In some embodiments, such as for treatments of plants, the topical formulation of a composition or pharmaceutical formulation described herein can be further formulated as a spray and can include a suitable surfactant, wetting agent, adjuvants/surfactant (stickers, extender, plant penetrant, compatibility agents, buffers, drift control additives, and defoaming agents), or any combination thereof so as to formulated as a spray. The compounds, any optional auxiliary active ingredient, suitable surfactant, wetting agent, adjuvants, or any combination thereof can be formulated as a solution, suspension, or emulsion. The spray dosage from can be administered through a spraying device. In some embodiments, the spraying device can be configured to generate the sprayable formulation as a liquid solution is contacted with the complexed active agent compound or formulation thereof. In other embodiments, the sprayable dosage form is pre-made prior to spraying. As such, the spraying device can act solely as an applicator for these embodiments.

In further embodiments, such as for treatments of plants (e.g. such as a herbicide), the dosage form of composition or pharmaceutical formulation described herein thereof can be further formulated as a dust and can include a suitable dry inert carrier (e.g. talc chalk, clay, nut hull, volcanic ash, or any combination thereof so as to be formulated as a dust. The dust can contain dust particles of varying sizes. In some embodiments, the particle size can be substantially homogenous. In other embodiments, the particle size can be heterogeneous. Dosage forms adapted as a dust can contain one or more adjuvants/surfactants (stickers, extender, plant penetrant, compatibility agents, buffers, drift control additives, and defoaming agents).

In some embodiments, the dosage form can be formulated as a bait. In these embodiments, the complexed active agent compound or other formulation thereof can be further formulated to include a food or other attractive substance that can attract one or more insect or other pest. The bait dosage form can be formulated as a dust, paste, gel, or granule. Dosage forms adapted as baits can contain one or more adjuvants/surfactants (stickers, extender, plant penetrant, compatibility agents, buffers, drift control additives, and defoaming agents).

In additional embodiments, the dosage form can be formulated as granules or pellets that can be applied to the environment. These dosage formulations are similar to dust formulations, but the particles are larger and heavier. The granules can be applied to soil or other environmental area. Dosage forms adapted as granules or pellets can contain one or more adjuvants/surfactants (stickers, extender, plant penetrant, compatibility agents, buffers, drift control additives, and defoaming agents).

The dusts, granules, and pellets described herein can be formulated as wetable dusts, granules, and pellets, soluble dusts granules, and pellets, and/or water-dispersible granules, and/or dry flowables.

The dosage form can be adapted for impregnating (saturating) an object or device, which then can be carried by, worn, or otherwise coupled to an organism in need thereof. In some embodiments, the dosage form can be impregnated onto a collar, bracelet, patch, adhesive tape, livestock ear tags, clothing, blankets, plastics, nets, and paints. The composition or pharmaceutical formulation thereof can be formulated and impregnated in the object or device such that the composition or pharmaceutical formulation evaporates over time, which releases the composition and/or pharmaceutical formulation into the air and/or environment surrounding the organism and/or onto the organism.

The dosage form can be adapted as a fumigant, which is a formulation that forms a gas when utilized or applied. In some embodiments, the composition and/or pharmaceutical formulation thereof can be supplied as a liquid when packaged under pressure and change to a gas when they are released. In other embodiments, the composition and/or pharmaceutical formulation thereof can be supplied as a volatile liquid when enclosed in a container (not under pressure). Others can be formulated as solids that release gases when applied under conditions of high humidity or in the presence of high water vapor. Dosage forms adapted as fumigants can contain one or more adjuvants/surfactants (stickers, extender, plant penetrant, compatibility agents, buffers, drift control additives, and defoaming agents).

Effective Amounts

The pharmaceutical formulations can contain an effective amount of a composition described herein and/or an effective amount of an auxiliary agent. In some embodiments, the effective amount ranges from about 0.001 μg to about 1,000 g or more of a composition described herein. In some embodiments, the effective amount of the composition described herein can range from about 0.001 mg/kg body weight to about 1,000 mg/kg body weight. In yet other embodiments, the effective amount of the composition can range from about 1% w/w to about 99% or more w/w, w/v, or v/v of the total pharmaceutical formulation. In some embodiments, the effective concentration of a polypeptide according to any one of SEQ ID NOs.: 1-9 can be between about 3 to about 10 mM.

Combination Therapy

The pharmaceutical formulations or other compositions described herein can be administered to a subject either as a single agent, or in combination with one or more other agents. Additional agents include but are not limited to DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepressants, and monoamine oxidase inhibitors), mebicar, afobazole, selank, bromantane, emoxypine, azapirones, barbituates, hyxdroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidio, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyttoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and 32-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscamet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecydine), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Screening Assays

The recombinant polypeptides and transformed cells expressing one or more of the polypeptides described herein can be used in an in vitro screening assay to screen candidate compounds to determine if they are capable of modulating binding of an ApoE protein to an ApoE receptor. As such, provided herein are methods including the steps of contacting a candidate compound with a recombinant polypeptide as described herein or an ApoE protein; and measuring binding of the recombinant polypeptide or the ApoE protein to an ApoE receptor present on the surface of a cell. In embodiments, the ApoE receptor is a LDL receptor. Measurement of the binding between the ApoE receptor and the ApoE protein or recombinant polypeptide as described herein can be conducted by measuring ApoE receptor activity.

In some embodiments, the amount and/or concentration of one or more Aβ peptides in cell cultured media can be measured. By measuring Aβ peptide production by the cell culture, candidate compounds including, but not limited to any small molecular compounds, small peptides, and antibodies, can be screened for their ability to specifically block ApoE binding to an ApoE receptor, such as LDLR. While not being bound to theory, measuring Aβ peptide production can be stimulated by ApoE binding to an ApoE receptor, such as LDLR. Therefore, if Aβ peptide production, as measured by the amount and/or concentration of one or more Aβ peptides in the cell culture media, is modulated in response to the presence of a candidate compound, it can be determined (in view of suitable controls) that the candidate compound influenced the binding of the ApoE to the ApoE receptor. In some embodiments, the binding of ApoE or a polypeptide as described herein to the ApoE receptor can be compared to a suitable control. One of ordinary skill in the art will instantly appreciate what constitutes a suitable control.

Methods of Treating a Cardiovascular, Vascular, and/or Neurologic Disease or Disorder

The compositions, recombinant polypeptides, and pharmaceutical formulations described herein can be useful for treating a disease or disorder where abnormal ApoE binding to an ApoE receptor is part of the pathology of the disease or disorder. As such, the compositions, recombinant polypeptides, and pharmaceutical formulations described herein can be useful for treating a cardiovascular, vascular, and/or neurological disease or disorder in a subject. The compositions, recombinant polypeptides, and pharmaceutical formulations described herein can be useful for treating AD.

In some embodiments, a compositions, recombinant polypeptide, or a pharmaceutical formulation described herein can be administered to a subject in need thereof. In some embodiments the composition or pharmaceutical formulation that can be administered to a subject in need thereof can include a polypeptide sequence according to SEQ ID NO: 1 or a polypeptide sequence that is about 90-100% identical to SEQ ID NO: 1 and at least 3 or at least 6 additional lysine residues operatively coupled to the polypeptide sequence according to SEQ ID NO: 1 or the polypeptide sequence that is about 90-100% identical SEQ ID NO: 1. In some embodiments the number of additional lysine residues can be 6. In some embodiments the recombinant polypeptide sequence can be any one of SEQ ID NOs: 1-9. The subject in need thereof can be suffering from or at risk for a disease or disorder where abnormal ApoE binding to an ApoE receptor is part of the pathology of the disease or disorder. The subject in need thereof can be suffering from or at risk for a cardiovascular, vascular, and/or neurological disease or disorder. The subject in need thereof can be suffering from or at risk for AD. In embodiments, ApoE binding to an ApoE receptor can be measured in the subject in need thereof. Methods for measuring specific binding of Apoe to an ApoE receptor can be determined by methods generally known in the art and will be instantly appreciated by those of skill in the art.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1. Apo3 and ApoE4, but not ApoE2, can Promote Amyloid p (an) Production

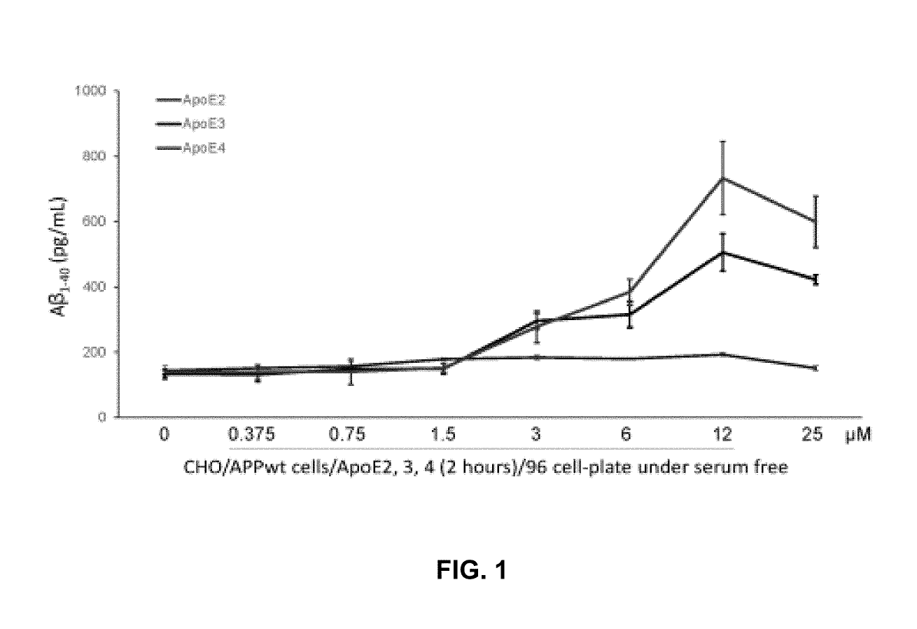

Chinese hamster ovary (CHO) cells overexpressing human wild-type amyloid precursor protein (APPwt) (CHO/APPwt cells) were cultured in 96-well plate at about 2×10⁴/well in Dulbecco's modified Eagle's medium (DMEM) with 10% fetal bovine serum (FBS), 1 mM sodium pyruvate and 100 U/mL of penicillin/streptomycin for 18 hours. These cells were then further cultured in serum-free DMEM in the presence or absence of ApoE2, ApoE3, or ApoE4 at various concentrations as indicated for about 2 hours, followed by analysis of conditioned media and cell lysates by Aβ₁₋₄₀ ELISA and protein assay, respectively. The results are shown in FIG. 1

As shown in FIG. 1, human LDL, ApoB100 or ApoE3 and 4 treatment can promote APP β-cleavage in human wild type (wt) APP expressing CHO cells (CHO/APPwt) as evidenced by (a) increased Aβ_{1-40, 42} production and (b) decreased sAPPα secretion.

### Example 2. ApoE LDLR Binding Domain Peptide Treatment can Increase Aβ Generation

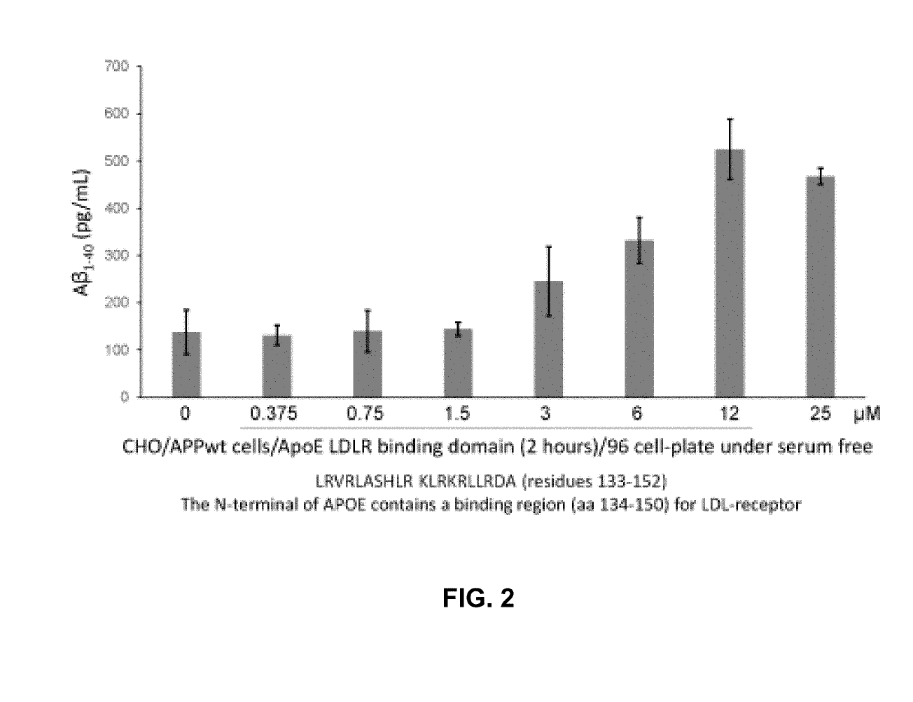

CHO/APPwt cells were cultured in DMEM with 10% FBS for about 18 hours and then serum-free DMEM in the presence or absence of ApoE LDLR binding domain peptide (LRVR LASH LRKL RKRL LRDA, residues 133-152 (SEQ ID NO: 1) as indicated for about 2 hours. The conditioned media and cell lysates were analyzed by Aβ₁₋₄₀ ELISA and protein assay, respectively. The results are shown in FIG. 2.

As shown in FIG. 2 peptide (LRVRLASHLRKLRKRLLRDA, SEQ ID NO: 1), representing the ApoE receptor binding region, promotes APP β-cleavage in CHO/APPwt cells as evidenced by (a) increased Aβ_{1-40, 42} production and (b) decreased sAPPα secretion.

### Example 3. ApoE LDLR Binding Domain Peptide Induced A8 Production is Structurally Dependant

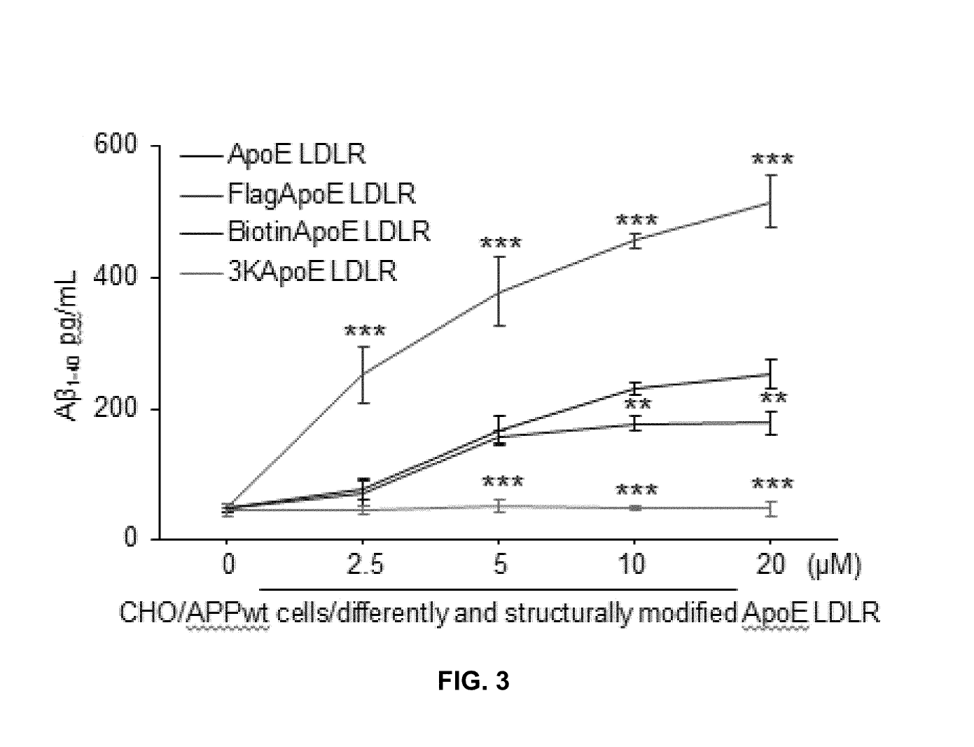

CHO/APPwt cells were cultured in 96-well plate (about 2×10⁴/well) in serum-free medium and treated with ApoE LDLR, FlagApoE LDLR (SEQ ID NO.: 1 with an N-terminal FLAG tag), BiotinApo LDLR (SEQ ID NO.: 1 with an N-terminal Biotin) and 3 lysinesApoE LDLR (3KApoE LDLR (SEQ ID NO.: 2)) at indicated doses for about 2 hours. The conditioned media and cell lysates were prepared, and subjected to Aβ ELISA (**P<0.01; ***P<0.001 when compared to ApoE LDLR) and protein assay. The results are demonstrated in FIG. 3 These results are representative of three independent experiments with each condition triplicated. Addition of 3-9 additional lysine residues at the N-terminal of the LDLR binding domain of ApoE (SEQ ID NO.: 1 LRVRLASHLRKLRKRLLRDA) can result in ApoE LDLR domain acting as an LDLR competitive antagonist to ApoE.

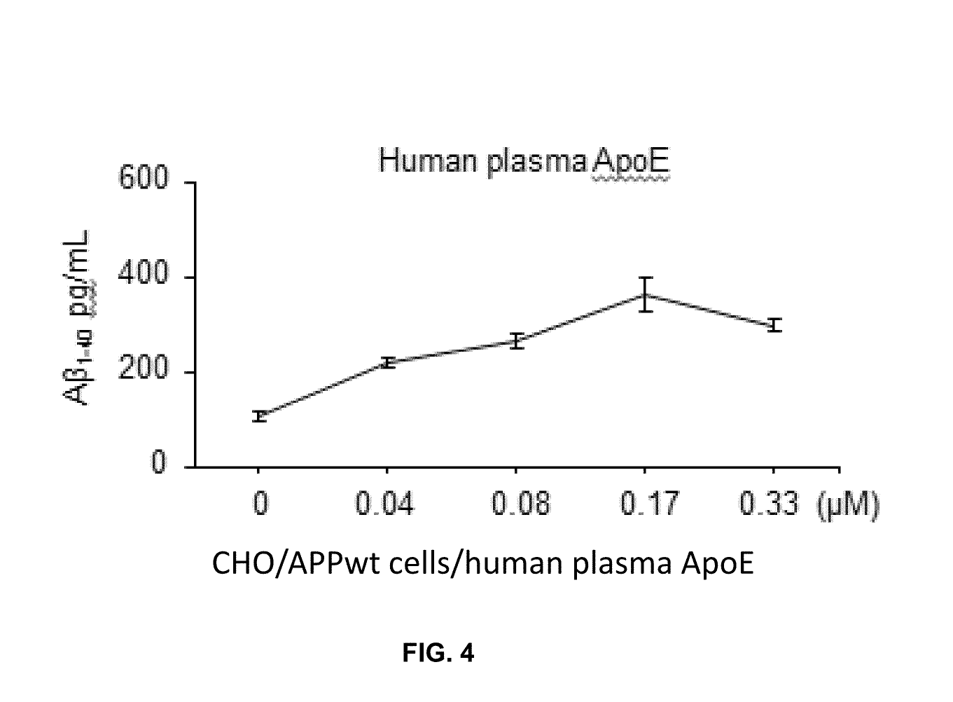

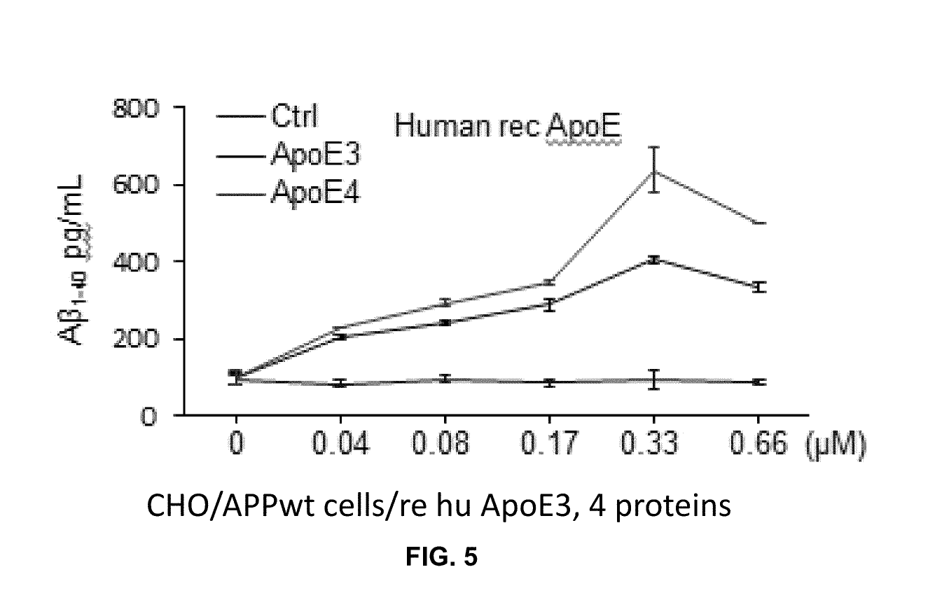

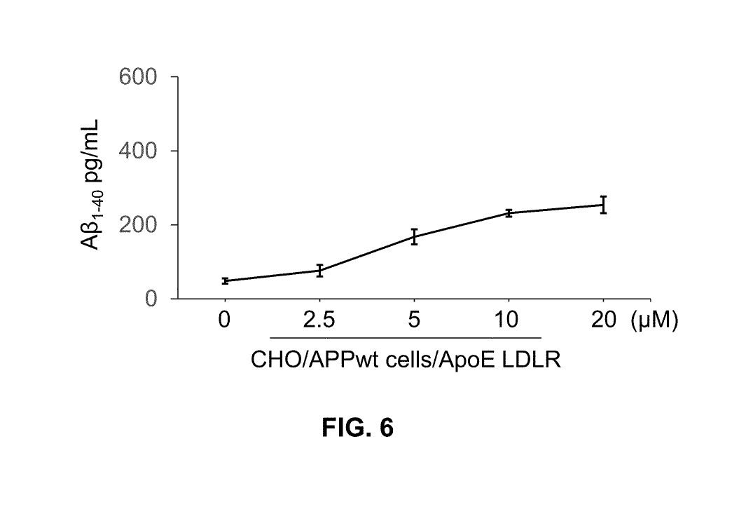

CHO/APPwt cells were cultured in 96-well plate (about 2×10⁴/well) in serum-free medium and treated with human plasma ApoE, human recombinant ApoE3, 4 (ApoE3, ApoE4) and ApoE LDLR as indicated doses and PBS (Ctrl) for about 2 hours. The conditioned media and cell lysates were prepared, and subjected to Aβ ELISA and protein assay. The results are demonstrated in FIGS. 4-6. These results are representative of three independent experiments with each condition triplicated and presented as mean±SD.

### Example 4. 3KApoE LDLR Markedly Inhibits Aβ Generation Induced by Human Plasma ApoE3, Human Recombinant ApoE3 and ApoE4, Human Plasma LDL, and ApoB100

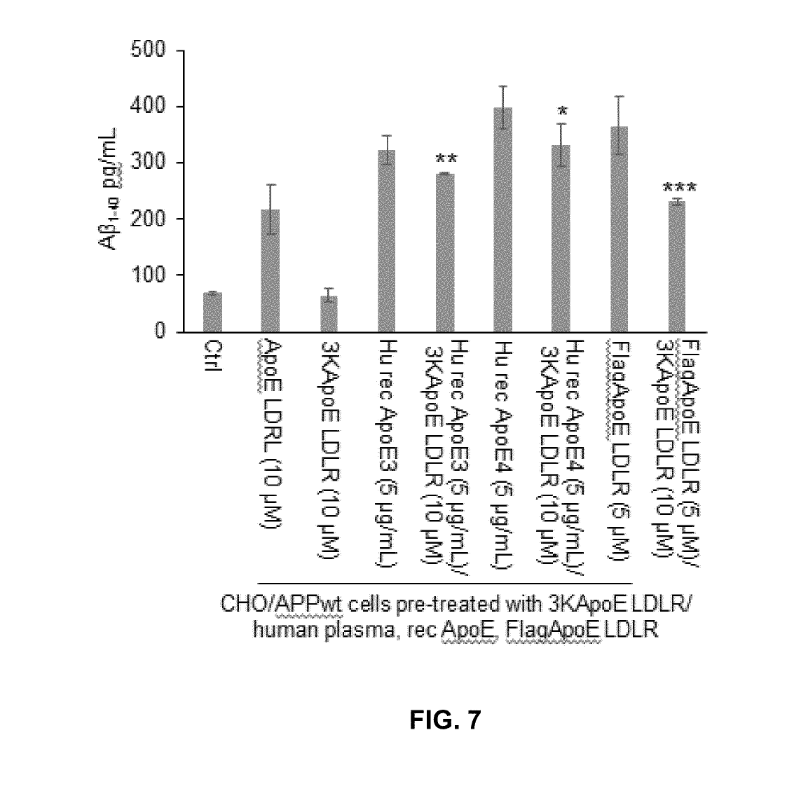

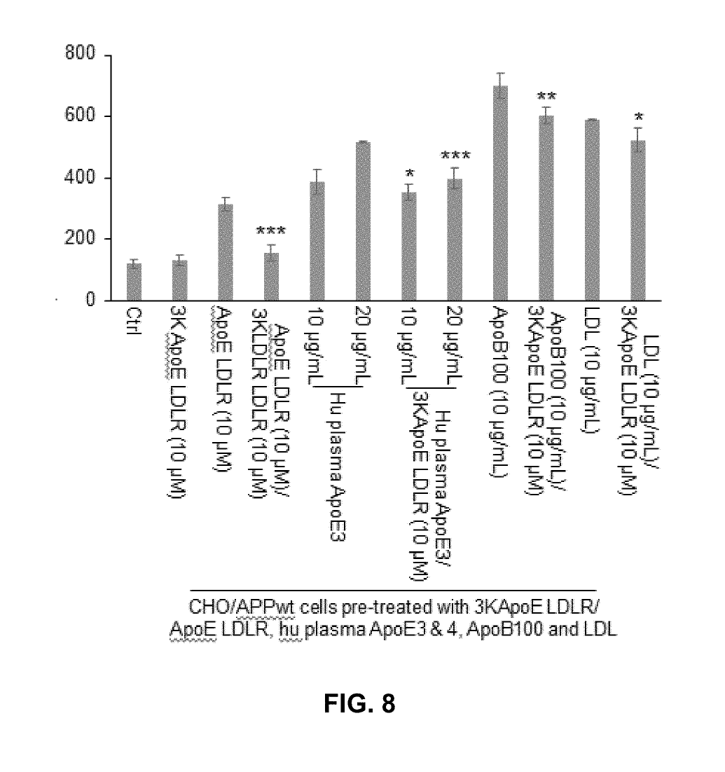

CHO/APPwt cells were cultured in 96-well plate (about 2×10⁴/well) in serum-free medium and pre-treated with 3KApoE LDLR (SEQ ID NO.: 2) at about 10 μM, followed by treatment with ApoE LDLR, human recombinant ApoE3, ApoE4, or FlagApoE LDLR (SEQ ID NO.: 1 with an N-Terminal FLAG tag) (results demonstrated in FIG. 7), human plasma ApoE3, ApoB100, or LDL (results demonstrated in FIG. 8) at indicated doses for about 2 hours. The conditioned media and cell lysates were prepared, and subjected to Aβ ELISA (*P<0.05, **P<0.01; ***P<0.001 when compared to control) and protein assay. These results are representative of three independent experiments with each condition triplicated and presented as mean±SD.

### Example 5. 6KApoE Markedly Inhibits Aβ Generation Induced by Human Recombinant ApoE4 Protein

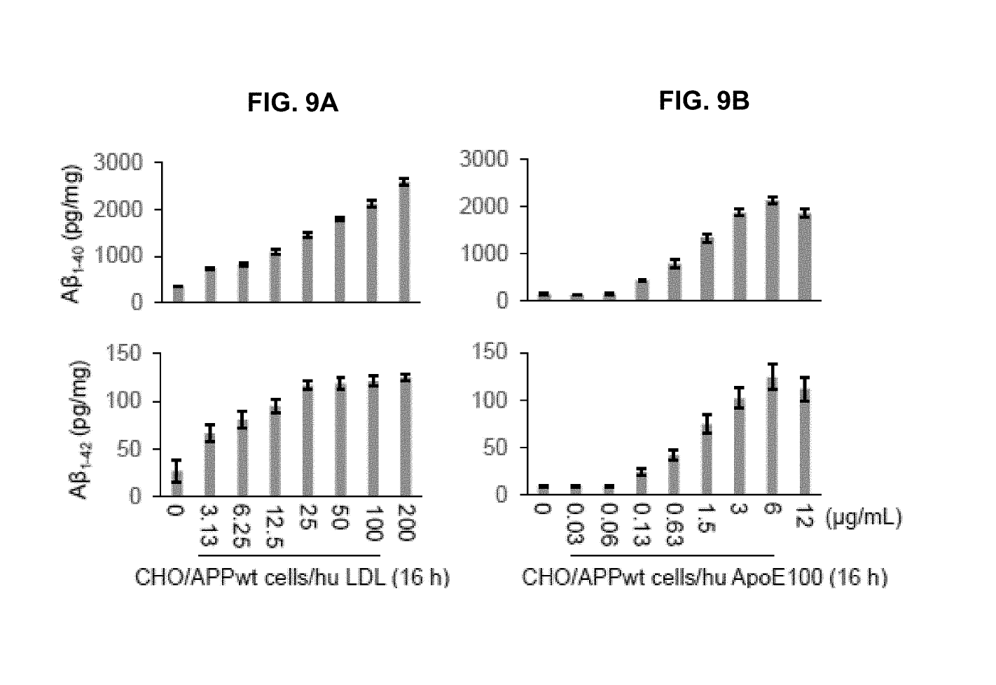

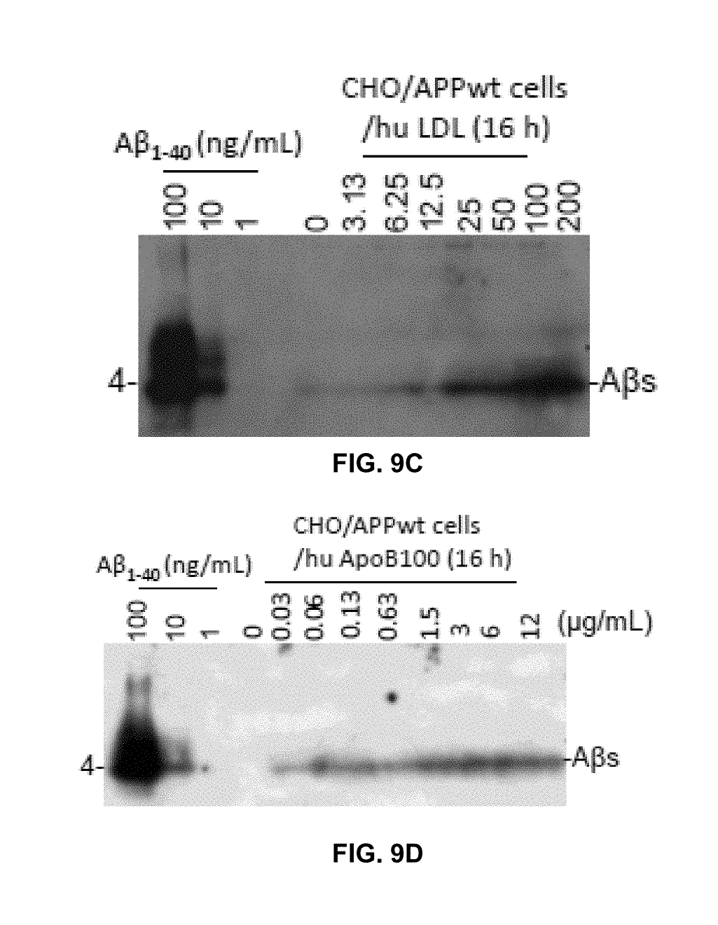

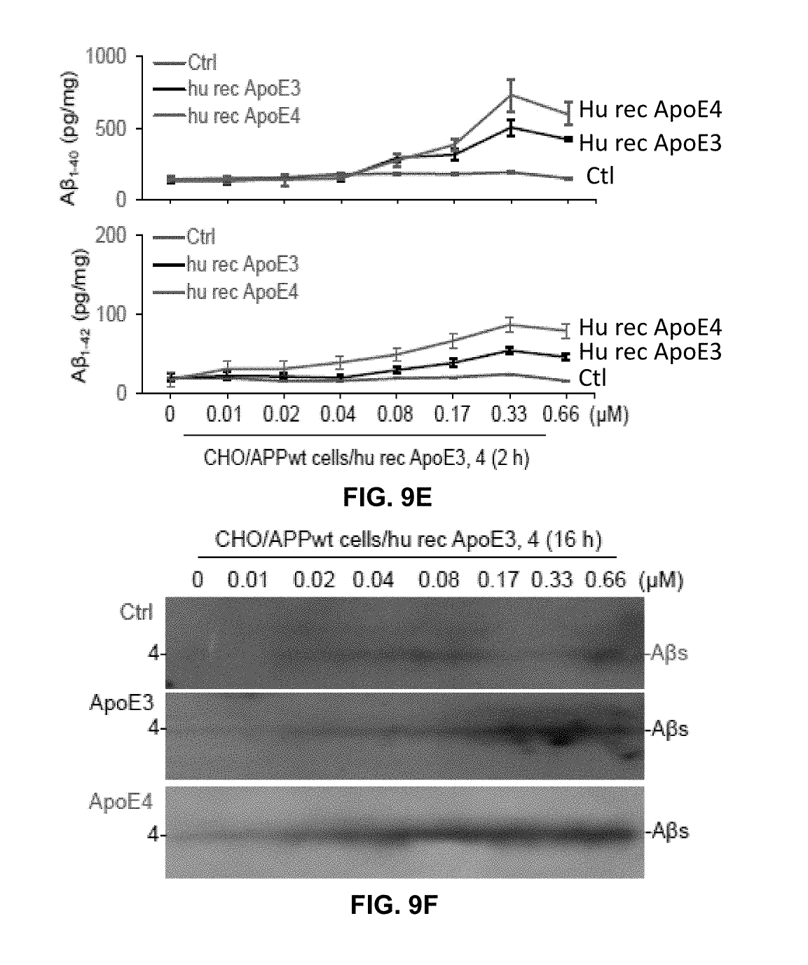

FIGS. 9A-9F show data demonstrating human plasma LDL, ApoB100 and human recombinant ApoE3 &4 proteins markedly promote amyloid β protein (Aβ) production. CHO cells overexpressing human wild-type APP (CHO/APPwt) cells were cultured in 96-well plate at 2×104/well in Dulbecco's modified Eagle's medium (DMEM) with 10% fetal bovine serum (FBS), 1 mM sodium pyruvate and 100 U/mL of penicillin/streptomycin for 18 hours. These cells were further treated with human plasma LDL (hu LDL) (FIGS. 9A and 9C), human plasma ApoB100 (hu ApoB100) (FIGS. 9B and 9D), human recombinant ApoE3, 4 protein (hu rec ApoE3 or 4) or PBS (Ctrl) (FIGS. 9E and 9F) at various concentrations in serum-free DMEM as indicated for 2 and 16 hours (h), followed by analysis of conditioned media and cell lysates by Aβ1-40, 42 ELISA, 82E1 western blotting (WB) analysis and protein assay, respectively. 82E1 WB analyses as shown below the Aβ ELISA result histograms. The Aβ ELISA results are representative of three independent experiments with each condition triplicated and presented as mean±SD.

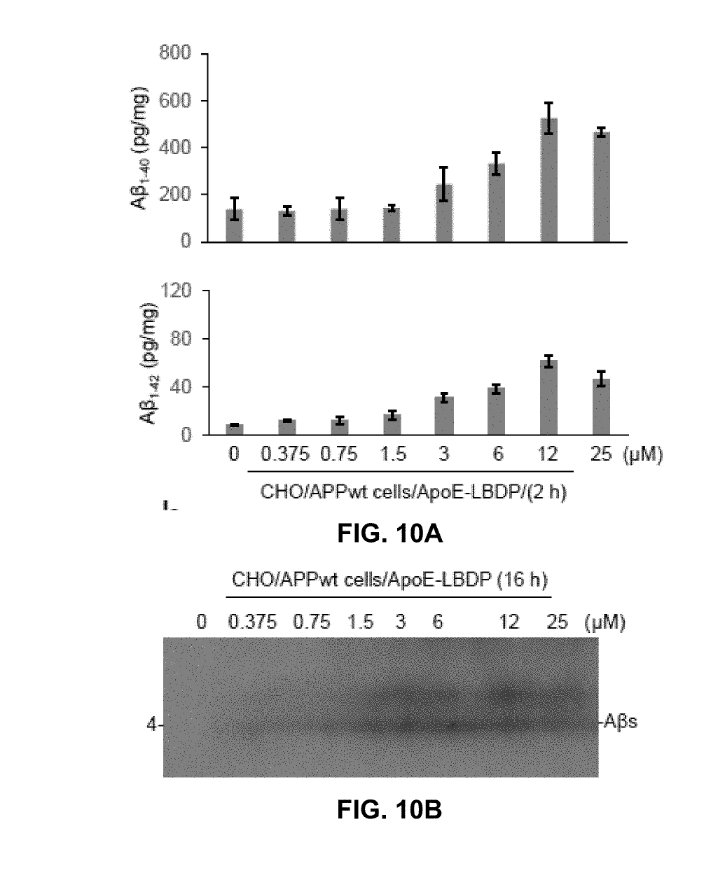

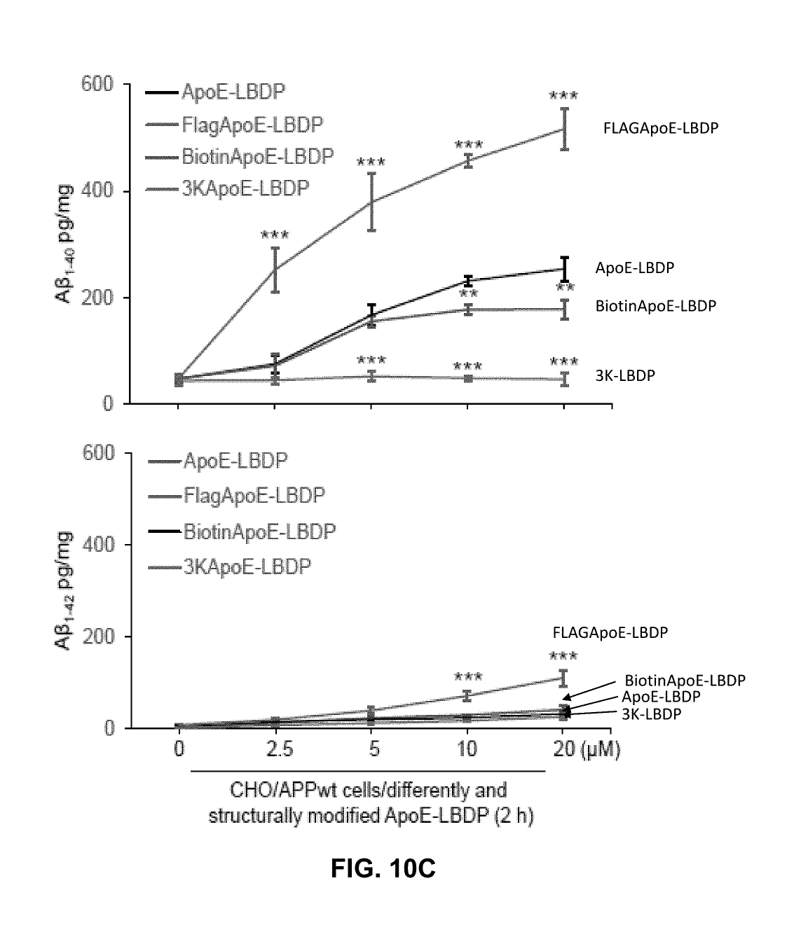

FIGS. 10A-10C show data demonstrating ApoE LDL receptor binding domain peptide treatment significantly increases Aβ generation, which is structurally dependant. CHO/APPwt cells were cultured in 96-well plate at 2×104/well in DMEM with 10% FBS for overnight and then serum-free DMEM in the presence or absence of ApoE LDL receptor binding domain peptide [ApoE-LBDP, LRVRLASHLRKLRKRLLRDA (residues 133-152, SEQ ID NO: 1)] as indicated for 2 and 16 hours (h). The conditioned media and cell lysates were analyzed by Aβ1-40, 42 ELISA (FIG. 10A), 82E1 WB analysis (FIG. 10B) and protein assay, respectively. In addition, (FIG. 10C) CHO/APPwt cells were cultured in 96-well plate (2×104/well) in serum-free DMEM and treated with FlagApoE-LBDP, BiotinApoE-LBDP and 3 lysineApoE-LBDP (3KApoE-LBDP) in comparing to ApoE-LBDP at indicated doses for 2 hours (h). The conditioned media and cell lysates were prepared, and subjected to Aβ1-40, 42 ELISA (**P<0.01; ***P<0.001 when compared to ApoE-LBDP) and protein assay. These results are representative of three independent experiments with each condition triplicated. Note: KKK-LRVRLASHLRKLRKRLLRDA (SEQ ID NO: 2); DYKDDDDK-LRVRLASHLRKLRKRLLRDA (SEQ ID NO: 10); Biotin-LRVRLASHLRKLRKRLLRDA (SEQ ID NO: 1 with an N-Terminal Biotin).

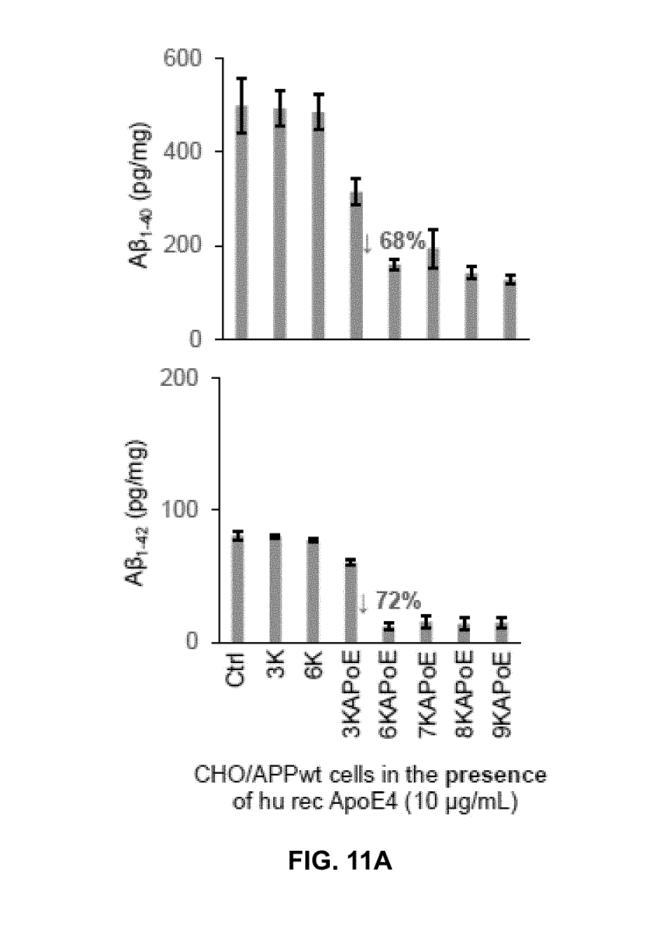

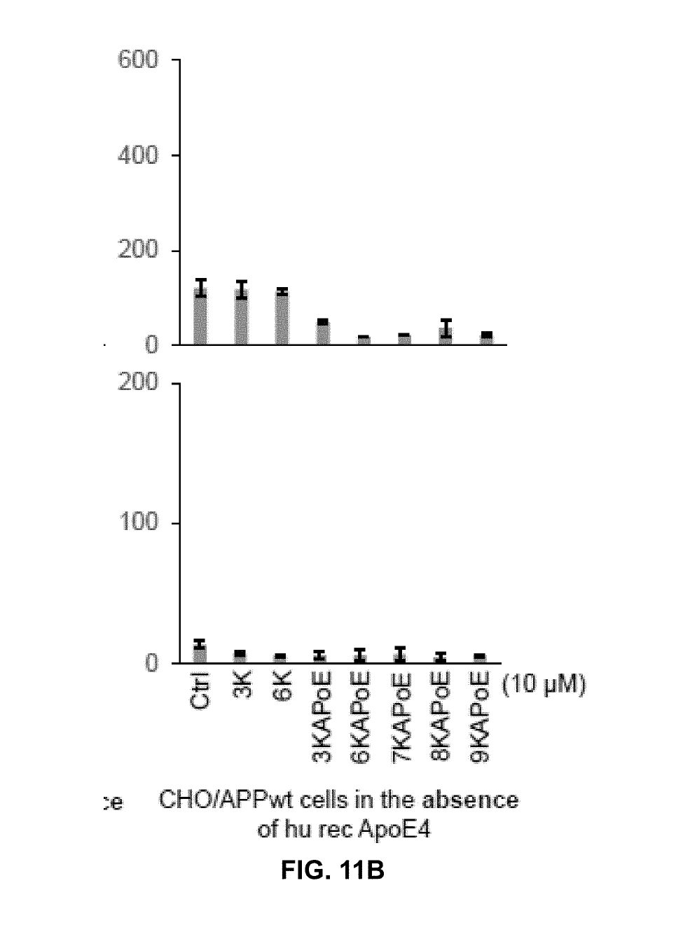

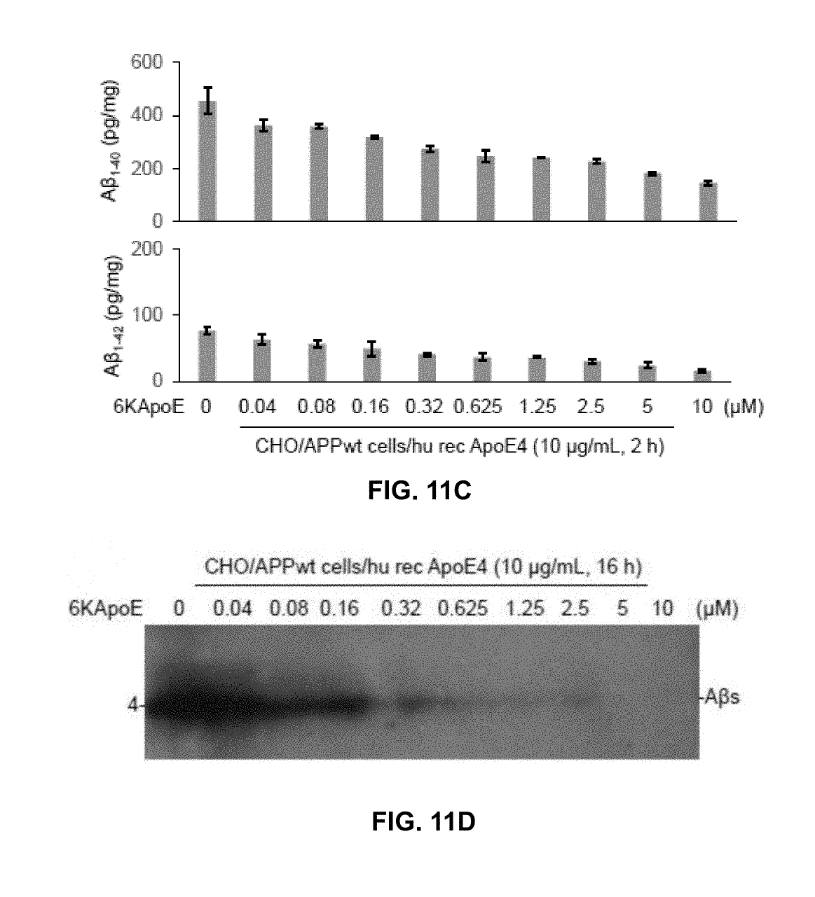

FIGS. 11A-11D show data demonstrating 6KApoE markedly inhibits Aβ generation induced by human recombinant ApoE4 protein. CHO/APPwt cells were cultured in 96-well plate (2×104/well) in serum-free medium and pre-treated with PBS (Ctrl), 3 lysine (3K), 6K lysine (6K), 3KApoE-LBDP (3KApoE) (SEQ ID NO: 2), 6KApoE-LBDP (6KApoE)) (SEQ ID NO: 5), 7KApoE-LBDP (7KApoE)) (SEQ ID NO: 6), 8KApoE-LBDP (8KApoE)) (SEQ ID NO: 7), 9KApoE-LBDP (9KApoE)) (SEQ ID NO: 8) peptide at 10 μM for 15 minutes and went to treatment with or without human recombinant ApoE4 protein (hu rec ApoE4, 10 μg/mL) for 2 hours. The conditioned media and cell lysates were prepared, and subjected to Aβ1-40, 42 ELISA and protein assay. Further, CHO/APPwt cells were cultured in 96-well plate (2×104/well) in serum-free DMEM and pre-treated with 6KApoE for 15 minutes at various doses as indicated and then went to treatment with human recombinant ApoE4 protein (hu rec ApoE4) for 2 and 16 hours. The conditioned media and cell lysates were prepared, and subjected to Aβ1-40, 42 ELISA (FIG. 11C), 82E1 WB analysis (FIG. 11D) and protein assay, respectively. The Aβ1-40, 42 ELISA results are representative of three independent experiments with each condition triplicated and presented as mean±SD.

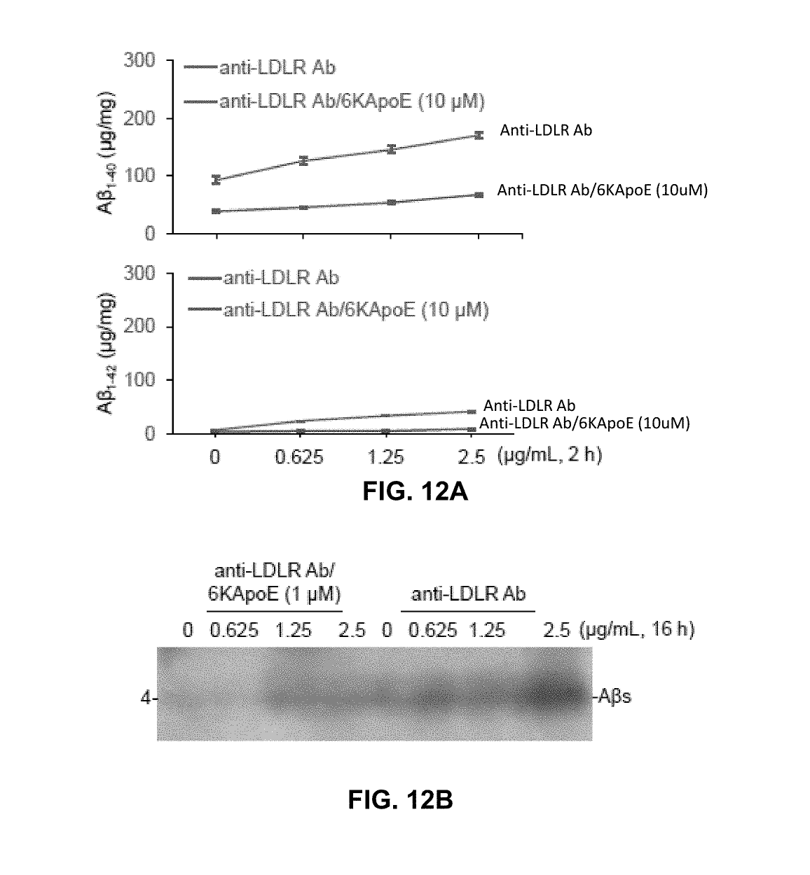

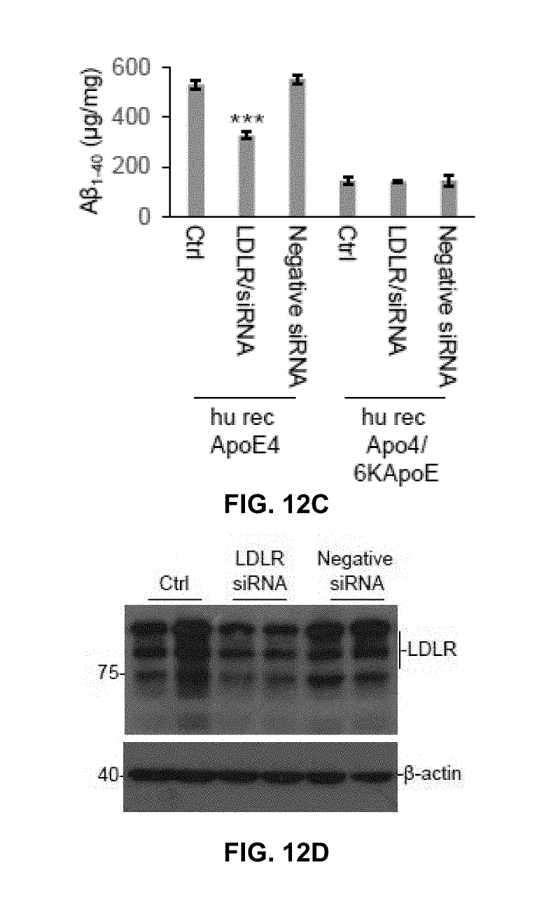

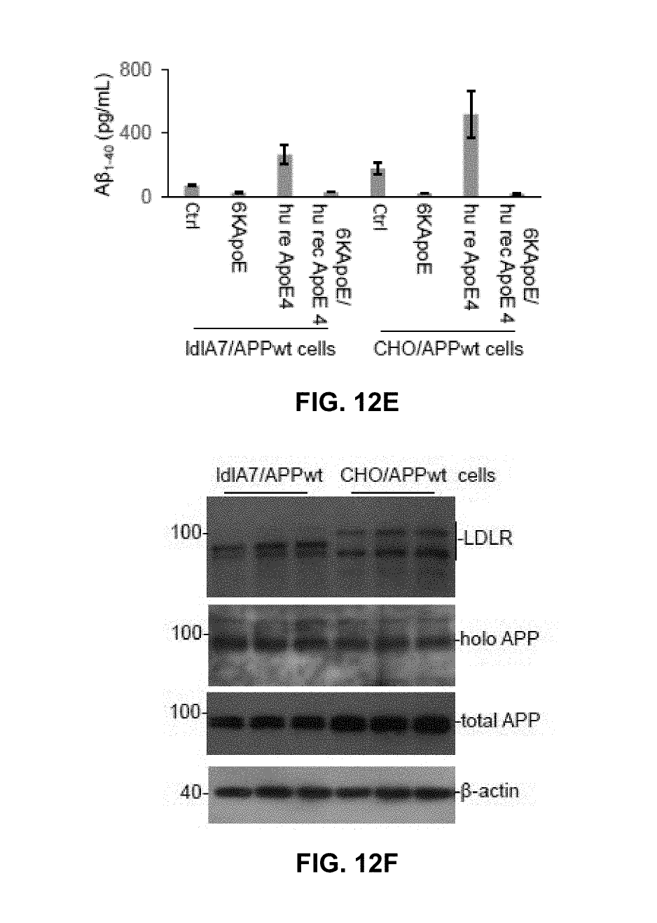

FIGS. 12A-12F show data demonstrating LDL receptor is in part responsible for 6KApoE inhibition of Aβ generation induced by human recombinant ApoE4 protein. CHO/APPwt cells were cultured in 96-well plate (2×10⁴/well) in serum-free medium and pre-treated with 6KApoE at 1 μM for 15 minutes and went treatment with an agonist anti-LDLR antibody (anti-LDLR Ab) from 0-2.5 μg/mL for 2 hours. The conditioned media and cell lysates were prepared, and subjected to Aβ1-40, 42 ELISA (FIG. 12A), 82E1 WB analysis (FIG. 12B) and protein assay. In addition, hamster LDLR siRNA and negative control were ordered from Thermo Fisher Scientific, transfection was performed following the protocol of lipofectamine RNAiMAX reagent provided by Thermo Fisher Scientific. Briefly, CHO APPwt cells were plated in 24 well plate 1×10⁵ cells/well for overnight, siRNAs were transfected at 10 nM as indicated, 24 hours after these cells were washed and treated with human recombinant ApoE4 protein (hu rec ApoE4) at 10 μg/mL for 2 hours (h) in the presence or absence of 15-minute pre-treatment of 1 μM 6KApoE. The conditioned media and cell lysates were prepared, and subjected to Aβ1-40, 42 ELISA (FIG. 12C), anti-LDLR antibody WB evaluation (FIG. 12D) and protein assay. CHO/IdlA7 (IdlA7) and CHO wild-type (CHO) cells were provided by Dr. Monty Krieger (Massachusetts Institute of Technology, Cambridge, Mass.). The two cells were cultured in Ham's F-12 medium supplemented with 5% FBS, 2 mM L-glutamine. The cells were plated into 24-well plate at 1×10⁵ each well the day before transfection. PCMV6-APP695 (OriGene Technologies, Inc. Rockville, Md.) was transfected to these cells using Lipofectamine) 3000 Transfection Reagent (Thermofisher Scientific) according to the instructions. Twenty-four hours after transfection, these cells were washed and treated with human recombinant ApoE4 protein (hu rec ApoE4) at 10 μg/mL for 2 hours (h) in the presence or absence of 15-minute pre-treatment of 1 μM 6KApoE. The conditioned media and cell lysates were prepared, and subjected to Aβ1-40 ELISA (FIG. 12E), and LDLR and APP WB evaluation (FIG. 12F), and protein assay. For Aβ ELISA, these results are representative of three independent experiments with each condition triplicated and presented as mean±SD.

### Example 6

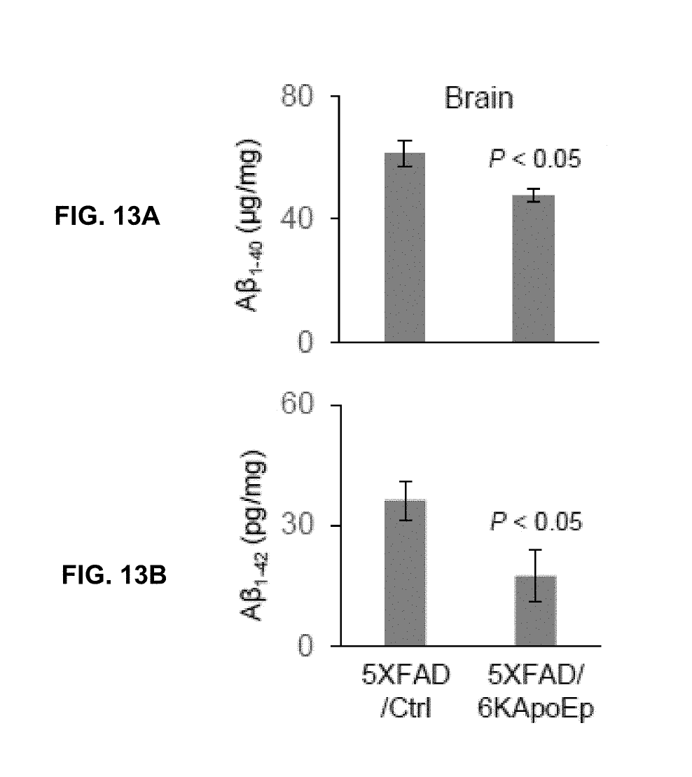

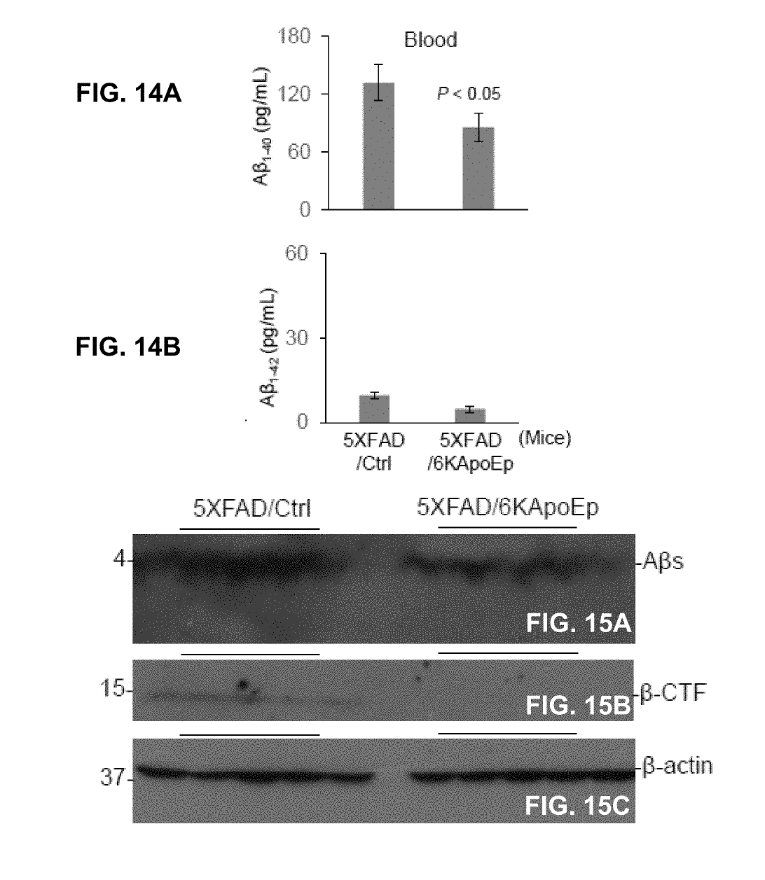

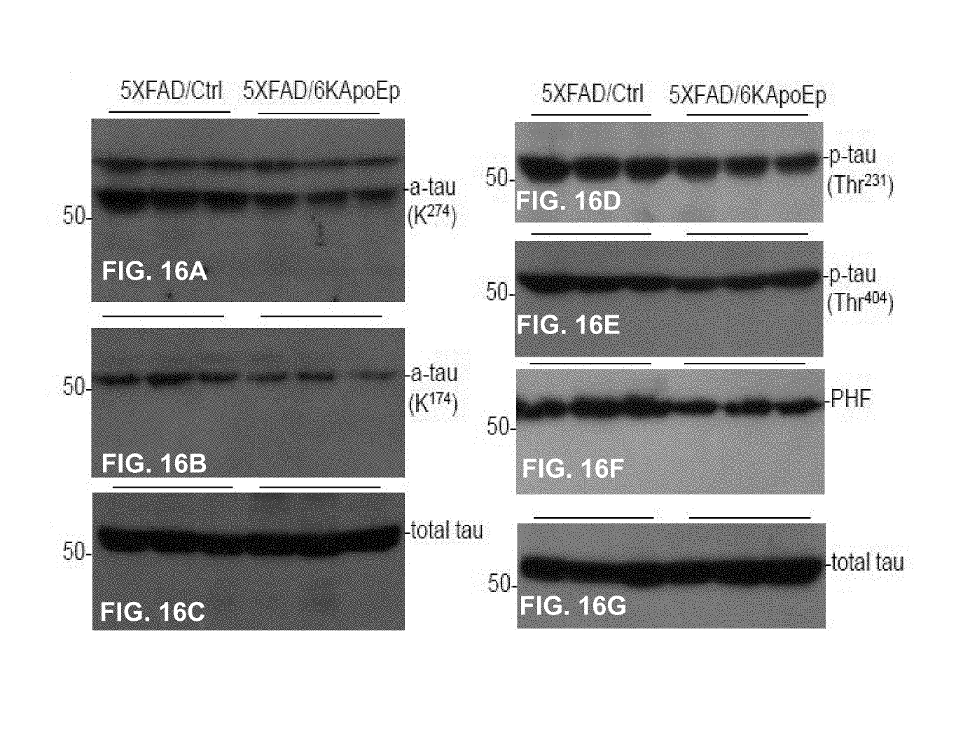

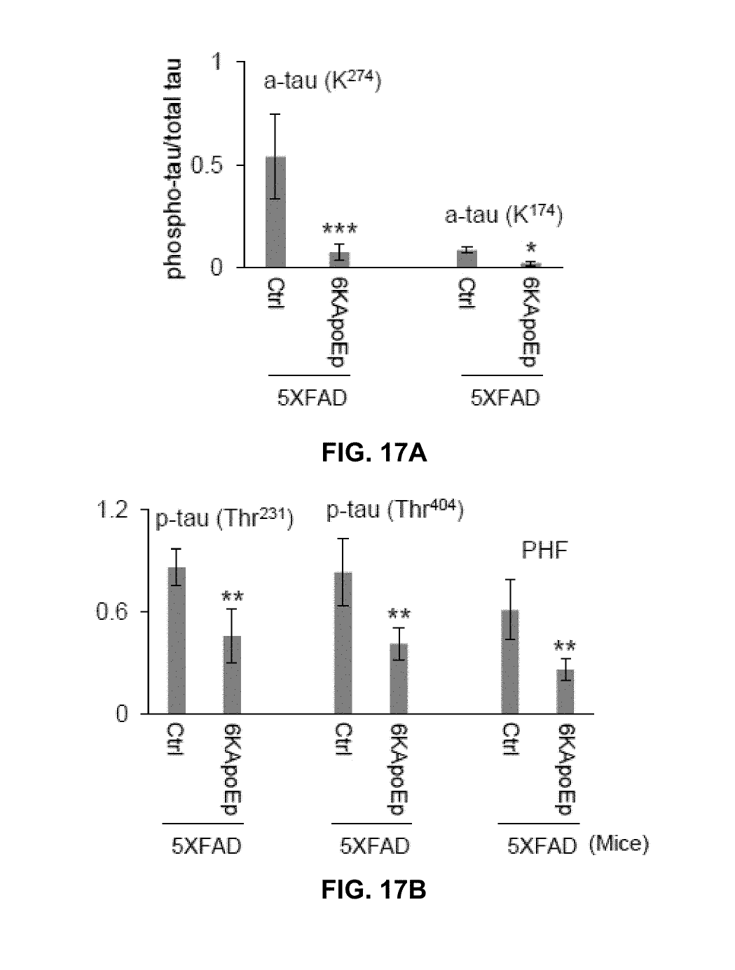

The effect of peripheral 6KApoEp treatment on Aβ levels and Alzheimer-like hyperphosphorylated and acetylated tau pathologies in an AD mouse model was examined. Briefly, 5×FADmice, with 3 APP and 2 PS1 AD mutations, at 6 weeks of age (n=10, 5female/5male) were intraperitoneally (i.p.) treated with 6KApoEp (250 μg/kg in 50 μL PBS) or PBS (50 μL) daily for 10 weeks. Following sacrifice, blood plasma and brain tissue homogenates were analyzed by Aβ_{1-40,42} ELISA (FIGS. 13A-13B and FIGS. 14A-14B) and Western blot for total Aβ, APP β-CTF (FIGS. 15A-15C) and Alzheimer-like acetylated (a-tau K²⁷⁴ and a-tau K¹⁷⁴) and phosphorylated tau (p-tau Thr²³¹, p-tau Thr⁴⁰⁴ and PHF (FIGS. 16A-16G). The ELISA results are represented as mean±SD (μg of Aβ1_{40,42} per total cerebral protein). Band density ratios of acetylated or phosphorylated tau to total tau as determined by densitometry analysis shown in FIGS. 17A-17B. Statistical t-test analyses of Western blot data revealed a significant decrease in the ratios of p-tau (Thr²³¹), p-tau (Thr⁴⁰⁴), PHF, a-tau(K²⁷⁴) and a-tau(K¹⁷⁴) total tau in 6KApoEp-treated compared with PBS injected 5×FAD mice (Ctrl,*P<0.05, **P<0.01, ***P<0.005).

### Example 7

Introduction.

Afflicting as many as 47 million people worldwide, Alzheimer's disease (AD) is characterized by the accumulation of amyloid-β peptide (Aβ) plaques and neurofibrillary tangles (NFTs) in the brain^{1,2}. Aβ is produced via β- and γ-secretase-mediated proteolysis of amyloid precursor protein (APP)^{3,4}, a type I transmembrane protein, which can then aggregate to form plaques. According to the amyloid hypothesis, overproduction of Aβ and its aggregation in senile plaques triggers neuronal apoptosis^{5,6}, inflammation^{7,8} and oxidative stress^{9,10}. In addition, the accumulation of Aβ can promote tau phosphorylation and aggregation in intracellular neurofibrillary tangles (NFTs)¹¹. Inherited early-onset familial AD (FAD) results from autosomal dominant mutations in APP or presenilin (PS) genes leading to excessive Aβ generation and neurodegeneration², lending support for the amyloid hypothesis. In the more common late-onset sporadic AD (SAD), excess Aβ generation is enhanced by age-related factors, metabolic dysfunction, cardiovascular disease and brain injury¹²⁻¹⁴. In addition, the apolipoprotein E4 (apoE4) allele has been found to be the major genetic risk factor for the development of SAD¹⁵. However, the pathogenesis of AD is still not completely understood and various therapeutic approaches targeting Aβ have failed, including Aβ-targeted immunotherapy. Thus, the relevance of Aβ in the pathogenesis of AD has been questioned.

ApoE, a major lipoprotein component of chylomicron remnants, very-low-density lipoproteins (VLDL), high-density lipoproteins (HDL) and intermediate-density lipoproteins (IDL) in the periphery, as well as of brain-derived lipoproteins, plays an important role in receptor-mediated cholesterol endocytosis^{16,17}. ApoE has three common alleles encoded by the APOE gene on chromosome¹⁹, which occur in different frequencies in the human population, namely ε2 (5-10%), ε3 (65-70%) and ε4 (15-20%). The proteins encoded by these genes differ by only two residues at positions 112 and 158. Thus, apoE2 has cysteine at both sites, apoE3 has a cysteine at site 112 and arginine at site 158, while apoE4 has arginine at both sites. Due to intramolecular folding, apoE3 and apoE4 have approximately 50-fold greater affinity for lipoprotein receptors than apoE2¹⁸. The presence of apoE4 dramatically increases the incidence of SAD, with one allele increasing the risk 3-fold and two alleles increasing the risk 12-fold¹⁵. In contrast, the presence of apoE2 is neuroprotective, while apoE3 is neutral^{19,20}. The cause for the enhanced risk associated with the presence of apoE4 is not yet clearly understood, but has been suggested to involve enhanced formation and reduced clearance of Aβ and the formation of neurotoxic apoE4 peptide fragments. In addition, apoE4 has been implicated in mediating abnormal tau phosphorylation, neuroinflammation and neurodegeneration, events which may be independent of increased Aβ production²¹⁻²⁴.

Previous studies indicated that apoE might potentially alter Aβ formation by interaction with apoE receptors such as low-density lipoprotein receptor (LDLR). However, APP can also act as a receptor for several ligands, based on its structural and functional similarities with other type I transmembrane receptors and γ-secretase substrates, including Notch and Deleted in the colorectal carcinoma (DCC) ²⁵⁻²⁷. Notably, after ligand binding to these receptors its proteolytic products mediate intracellular signaling and transcription involved in neuronal outgrowth, synaptogenesis and axon guidance. APP in particular has two extracellular heparin binding domains which bind negatively charged molecules such as heparin and matrix proteins, one of high affinity named E1 or growth factor-like domain (GFLD) located at the N-terminus of APP and another of lower affinity named E2 located proximal to the membrane. Disulfide bridges between adjacent APP molecules at the E1 domain resembles other receptor-like proteins, appears to stabilize the APP structure at the cell surface and favors ligand induction of signaling cascade ^{28,29}. In addition, the E2 domain can bind albeit with low affinity to membrane-anchored heparan sulfate proteoglycans (HSPGs), which can act as a co-receptor and allow putative ligands to bind with higher affinity ^{30,31}. However, unlike the case of Notch and DCC, the putative ligand(s) of APP has never been characterized.

Early studies utilizing yeast two-hybrid and immunoprecipitation of cell culture supernatants suggested that the N-terminus of APP can directly bind apoE³². The domain where interaction between APP and apoE occurred in these experiments was reported to be the amino-terminal region of APP, between amino acids 1-207 and upstream of the Aβ region, and the amino-terminal region of apoE, between amino acids 1-191. In addition, apoE enhanced intracellular APP endocytosis and/or retention and reduced sAPPα production. More recent studies indicate that both glia-derived and recombinant apoE stimulates Aβ production in embryonic stem cell-derived human neurons with a rank order of potency of apoE4>apoE3>apoE2, mediated by a signal transduction cascade involving activation of a non-canonical mitogen associated protein kinase (MAPK) p44/p42 and APP transcription/translation ³³. Altogether these studies suggest that apoE can bind to the N-terminal region of APP, thereby enhancing APP endocytosis and directing its processing from sAPPα to Aβ production. Importantly, upon ligand binding, the intracellular region of APP can also directly interact with a host of other signaling cascades, notably those involving heterotrimeric Go proteins ³⁴ as well as increased production of the APP Intracellular Domain (AICD) ³⁵, which can mediate many aspects of AD pathogenesis, including tau hyperphosphorylation, neuronal inflammation and neurodegeneration. It is not understood if apoE binding to APP elicits activation of these signaling mechanisms, thereby playing a role in AD pathogenesis.

In this Example, the role of apoE binding to the N-terminus of APP in Aβ production and AD pathology was examined. It was confirmed that the LDLR binding domain of apoE physically interact with the N-terminus of APP in vivo and in vitro. Antibodies directed against the N-terminus of apoE or APP as well as truncation of the N-terminal region of APP reduced apoE-mediated Aβ production, confirming that Aβ production is elicited by this interaction of apoE with N-terminus of APP. Finally, a peptide antagonist of apoE-APP interaction was generated, which was observed to reduce cerebral Aβ and tau pathologies, neuronal apoptosis, synaptic loss and cognitive impairment in 5×FAD mice, presumably in part through decreasing APP cellular membrane trafficking, p44/42 MAPK phosphorylation and subsequent Aβ production. Taken together, these data suggest that disruption of apoE interaction with the N-terminus of APP may be a disease-modifying therapeutic strategy for AD.

Results.

6KApoEp Treatment was Observed to Markedly Suppress Human Recombinant and Lipidated apoE-Induced Aβ Production.

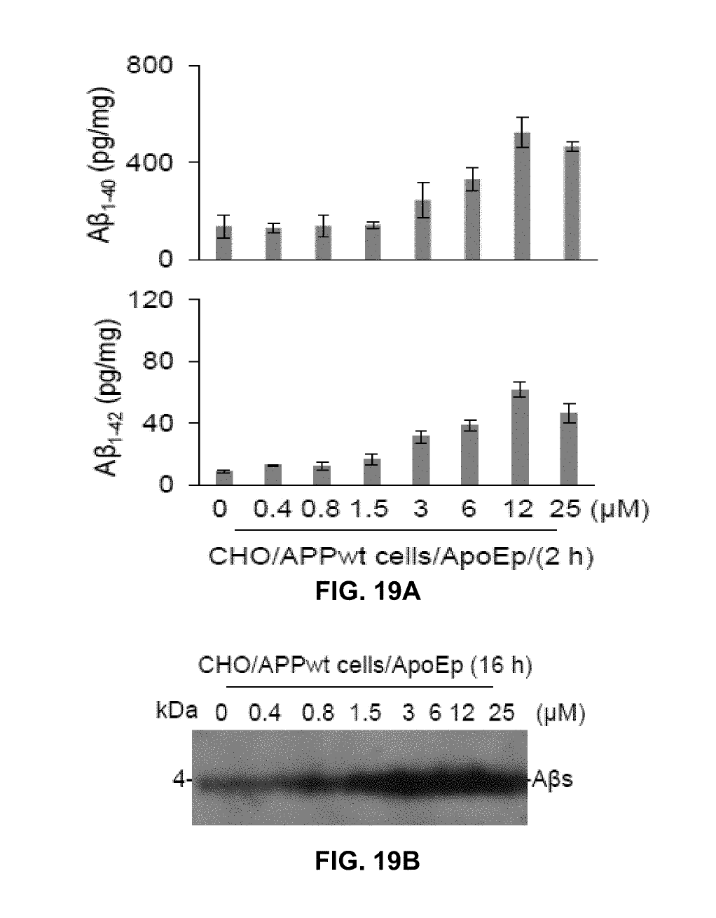

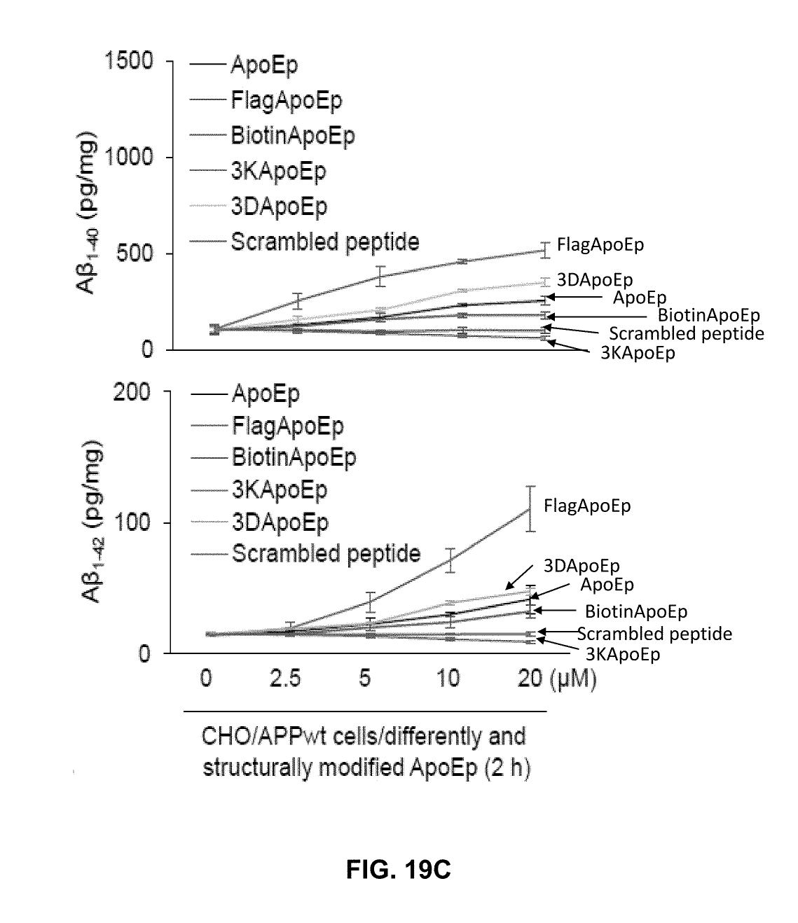

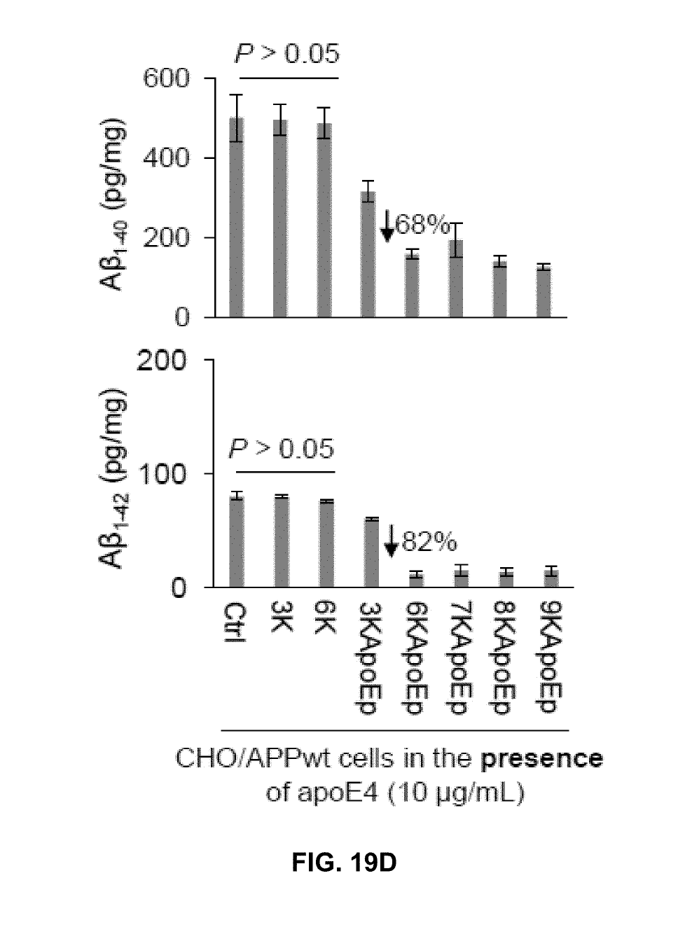

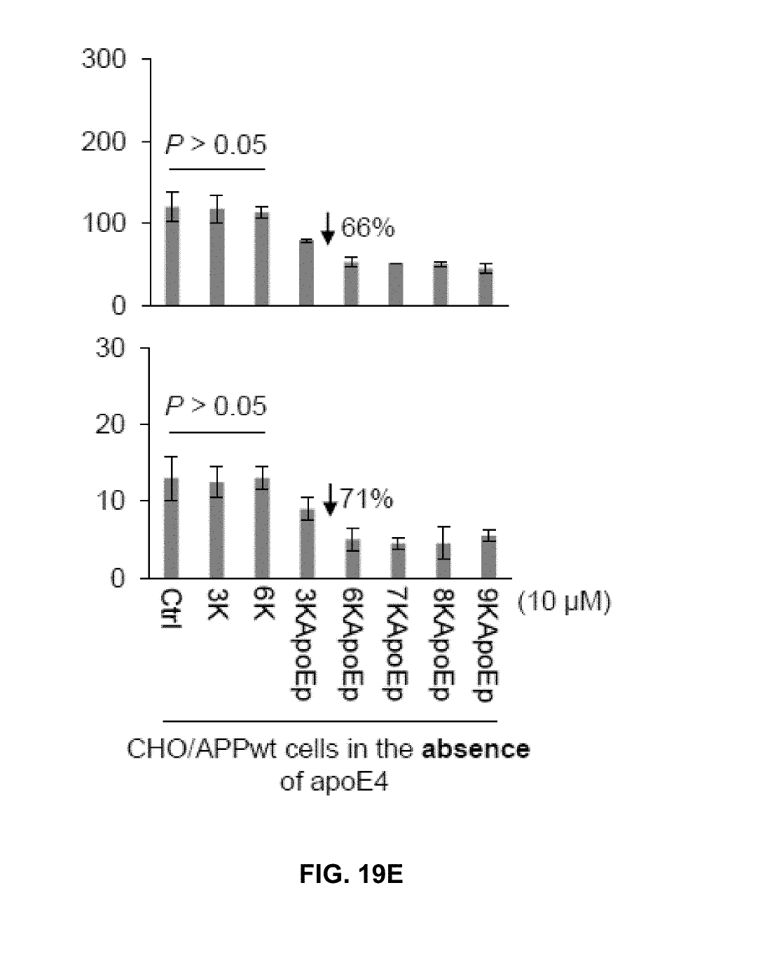

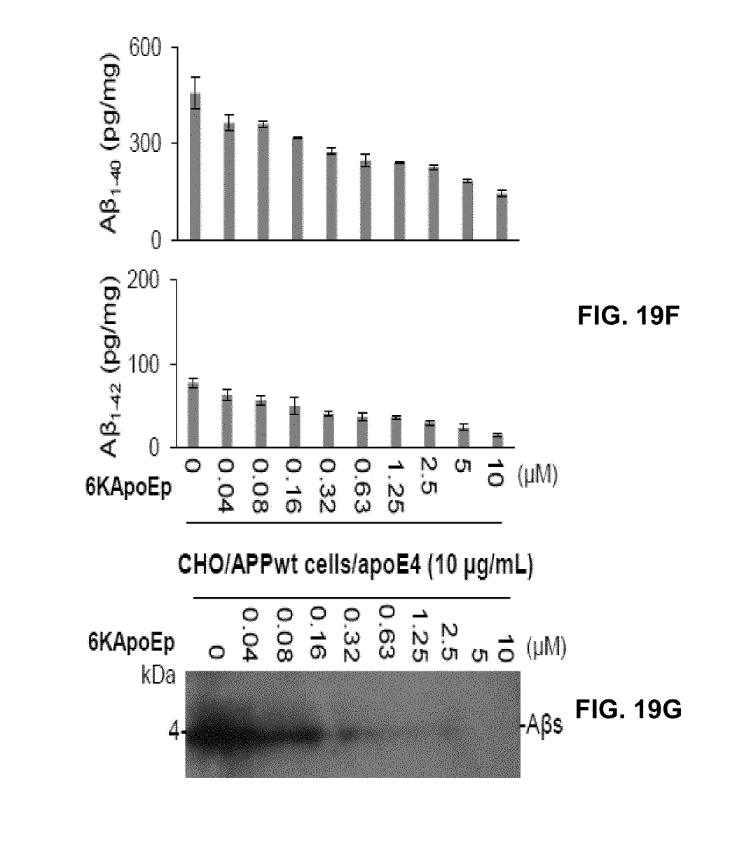

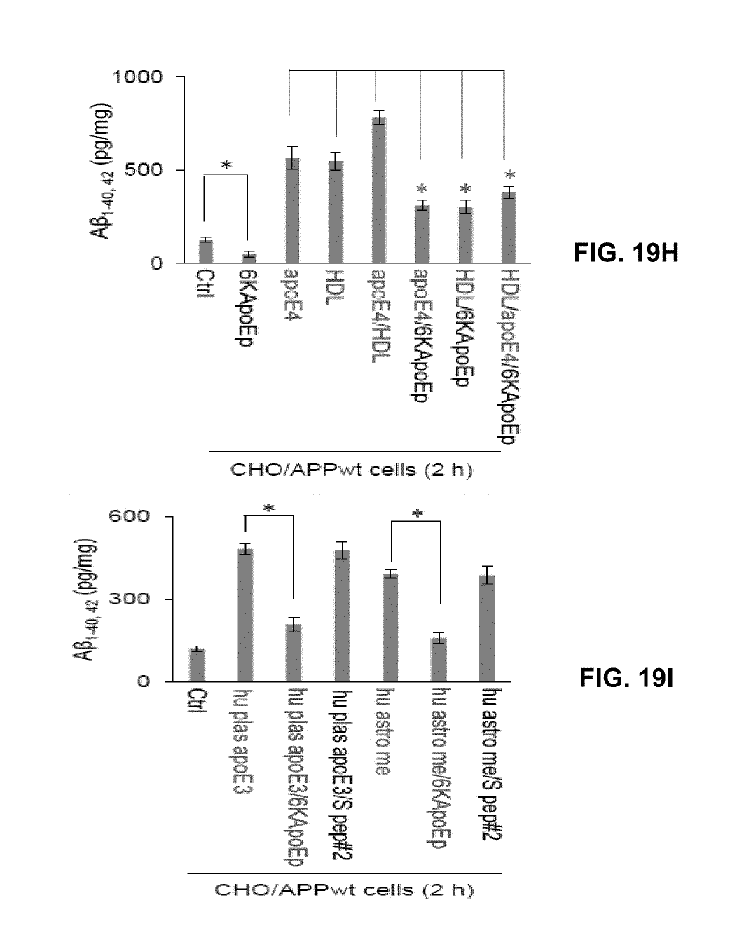

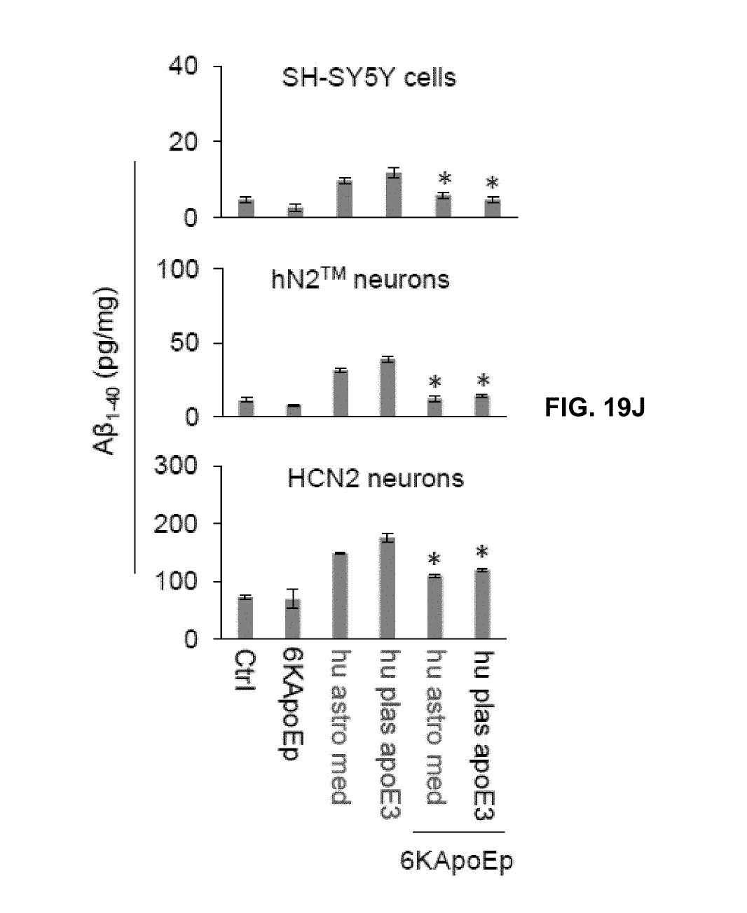

Previous findings suggest that apoE4 exacerbates AD pathology, in part, by enhancing APP amyloidogenesis ³³. In addition, the amino-terminal region of apoE (residues 133-152) is known to contain the apoE receptor binding domain, while structural modifications of apoE are known to modify intramolecular interactions and thereby mediate differential interaction of apoE isoforms with its receptor³⁶. In order to determine the specific region of apoE mediating Aβ production, as well as further investigate the effects of structural modifications of apoE, the apoE LDLR binding domain and proximal structural modifications were focused on. As expected, an apoE peptide (ApoEp, apoE mimetic), consisting only of the binding domain of apoE (residues 133-152), markedly increased Aβ production in a concentration-dependent manner in CHO/APPwt cells (FIGS. 19A-19C). While amino terminal addition of Flag-tag greatly enhanced the efficacy of ApoEp to increase Aβ1-40, 42 levels, the addition of three lysine residues (3K) terminated this amyloidogenic effect (FIG. 19C).

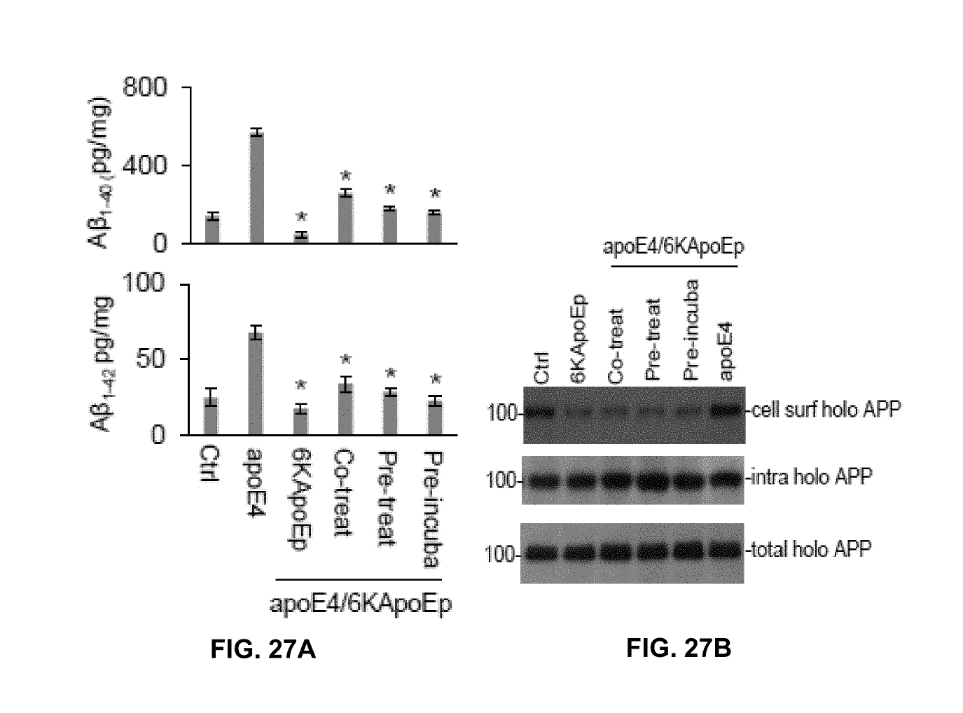

To test the hypothesis that the addition of amino terminal lysine residues might convert ApoEp to an apoE antagonist, we examined the effects of ApoEp containing 3, 6, 7, 8 or 9 lysines on apoE4-induced Aβ production. 3KApoEp moderately while 6-9KApoEp markedly and maximally reduced both basal and apoE4-induced Aβ production (FIGS. 19D-19E). In addition, 6KApoEp reduced apoE4-induced Aβ production in a concentration-dependent fashion, starting at 40 nM and with an IC50 of approximately 0.32-0.63 μM (FIG. 19F-19G). This reduction was observed regardless of whether the cells were pre-treated with 6KApoEp, co-treated with 6KApoEp together with apoE4, or treated with apoE4 pre-incubated with 6KApoEp (FIGS. 27A-27B). 6KApoEp also reduced Aβ production elicited by HDL lipidated apoE4, human plasma derived apoE3 and human astrocyte media derived apoE in CHO/APPwt cells, SH-SY5Y human neuroblastoma cells and hN2TM and HCN2 human neurons (FIGS. 19H-19J), confirming that 6KApoEp reduces natural apoE-mediated Aβ production.

6KapoEp Reduces Physical Association of apoE with N-Terminus of APP.

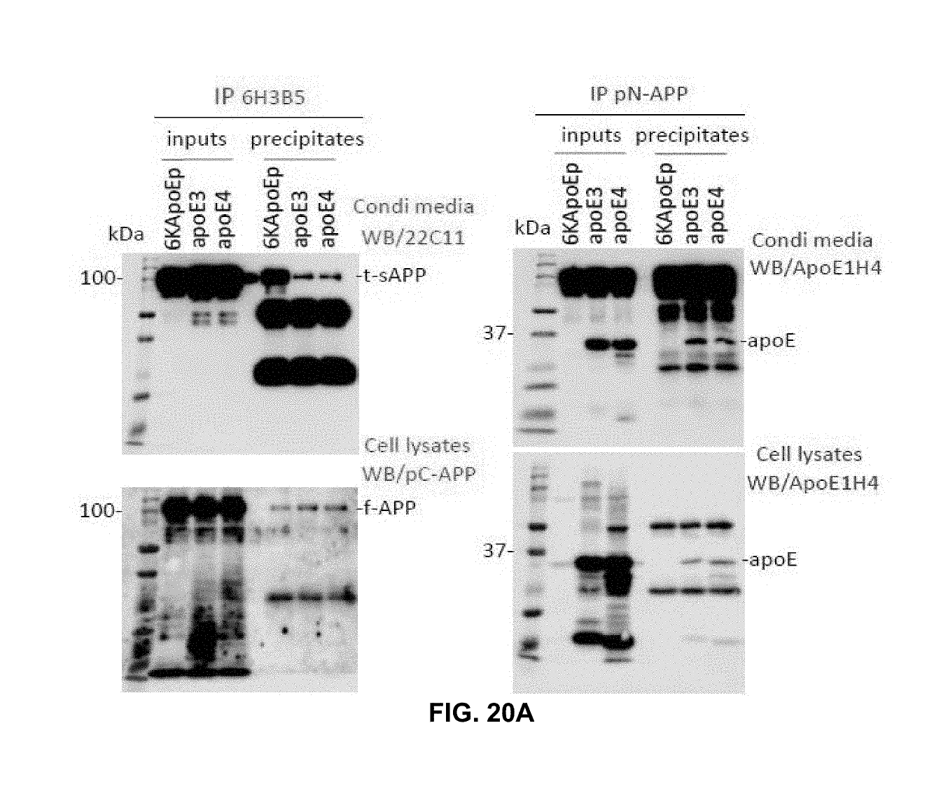

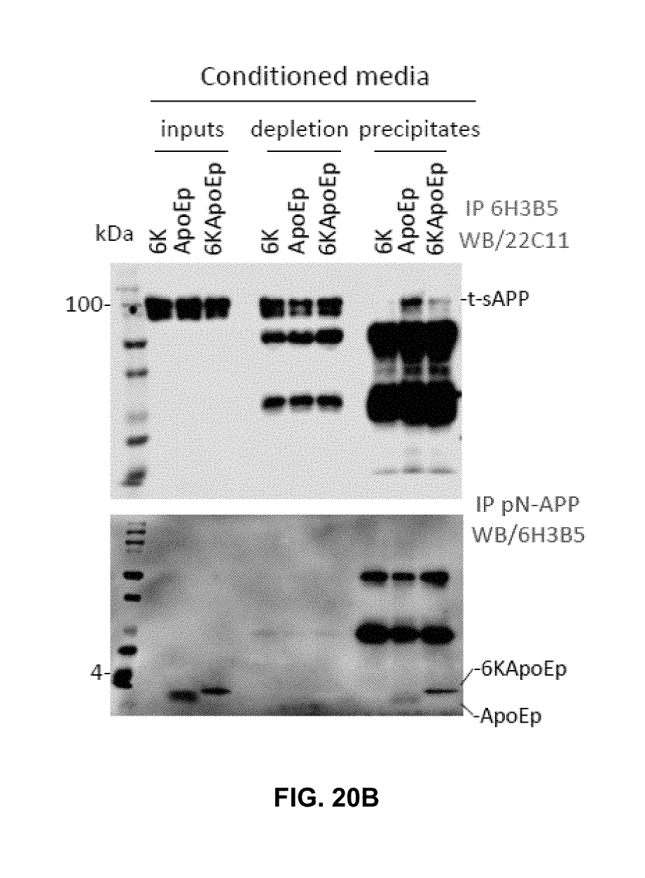

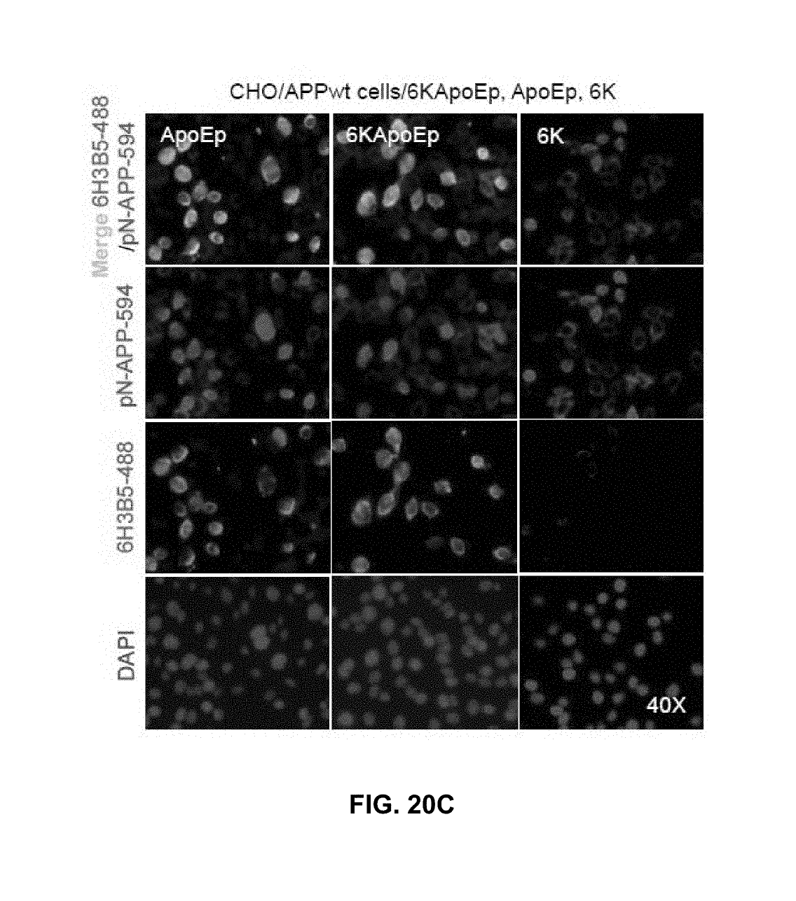

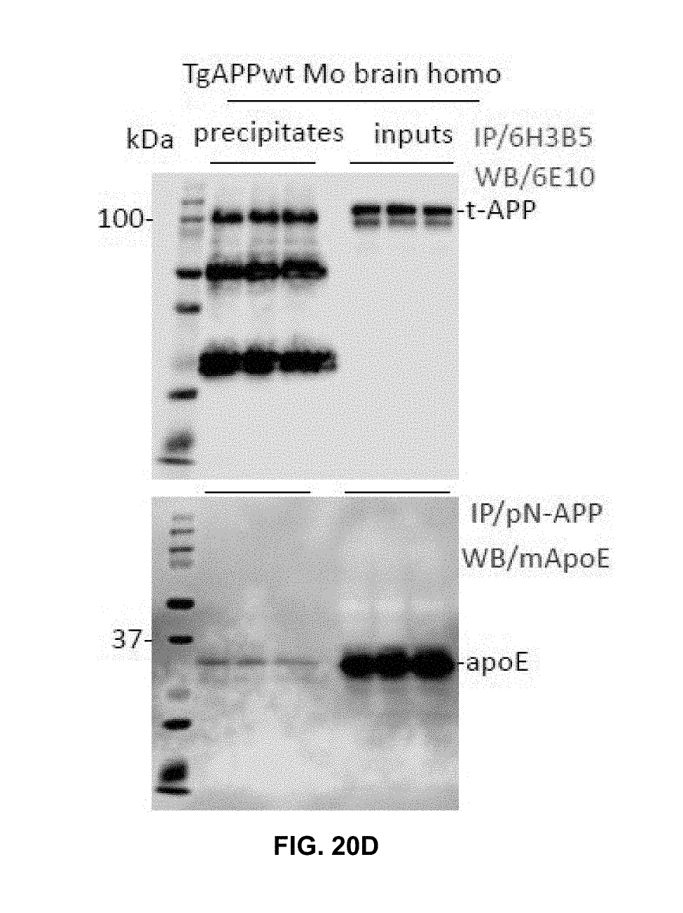

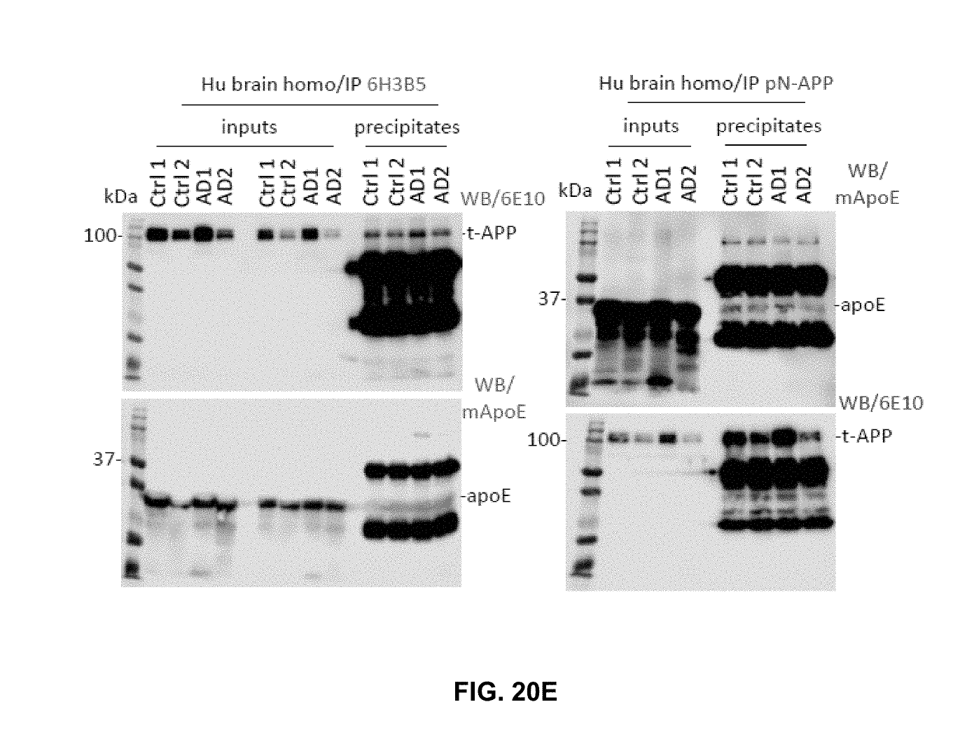

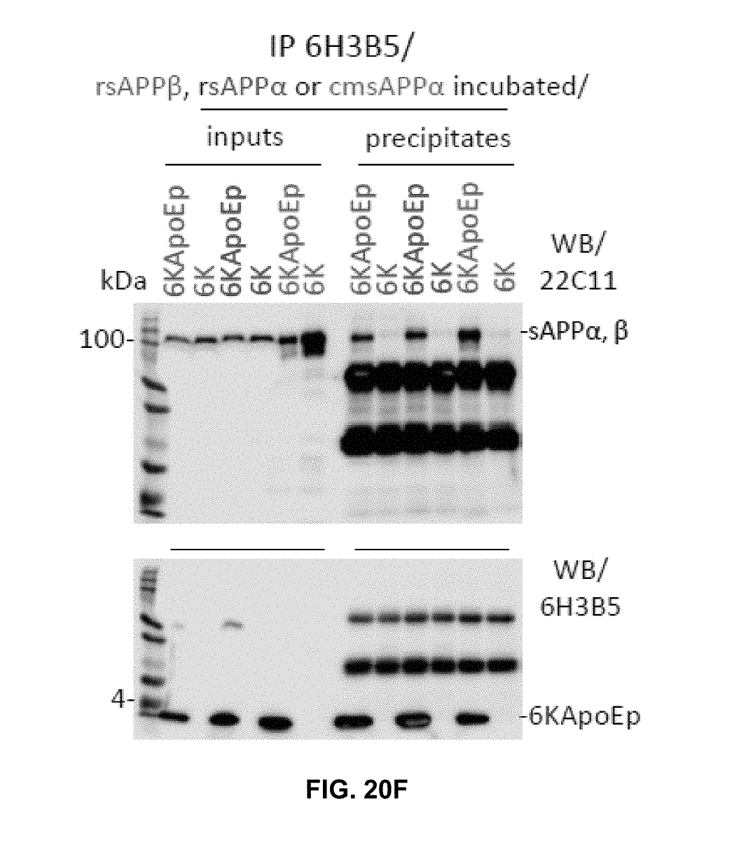

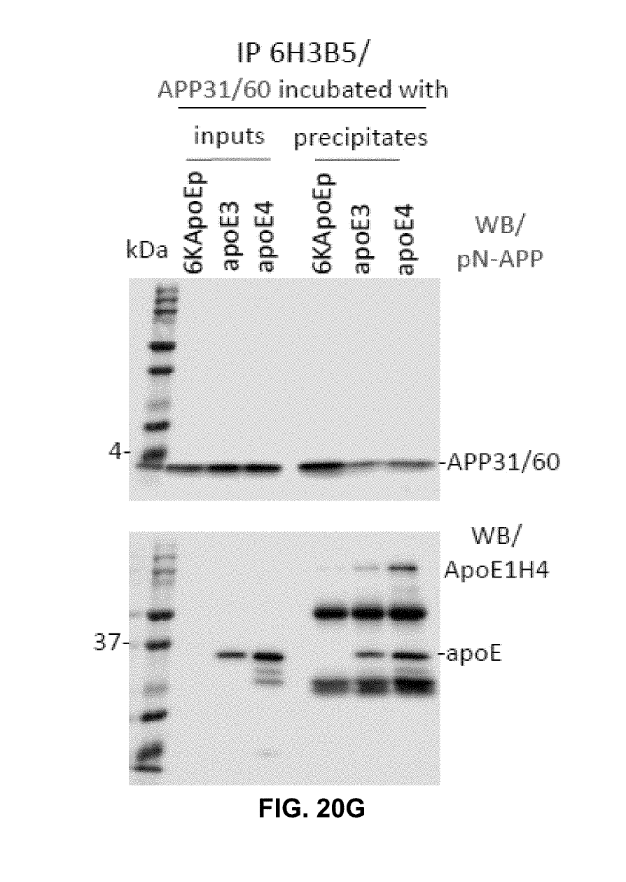

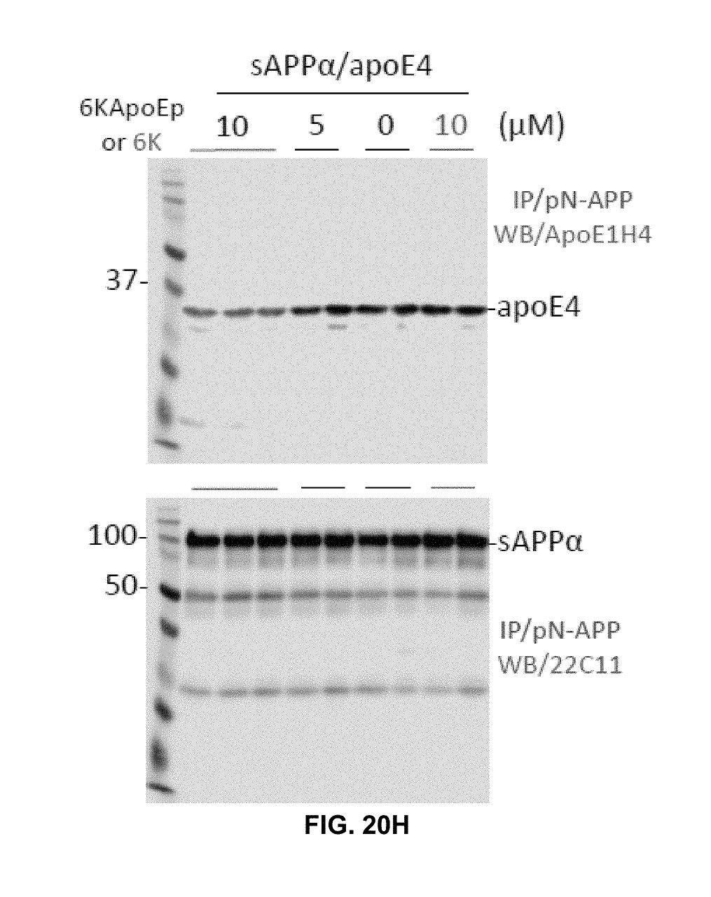

Previously, apoE was found to bind to the N-terminus of APP, upstream of the Aβ region, and thereby enhance APP endocytosis and reduce sAPPα production³². In order to confirm this physical association of apoE with N-terminal APP, CHO/APPwt cells were treated with ApoEp, 6KApoEp, 6K, apoE3 or apoE4 followed by immunoprecipitation (IP) of apoE with LDLR binding domain antibody (6H3B5). Total secreted APP in conditioned media and full length APP in cell lysates were then determined by WB using anti-N-terminal (22C11) and anti-C-terminal APP antibodies (pC-APP), respectively. Alternatively, N-terminal APP was IP with anti-N-terminal APP41/66 antibody (pN-APP) followed by analysis of apoE, 6KapoEp and ApoEp using anti-human apoE (ApoE1H4) and 6H3B5 antibodies. ApoE, ApoEp and 6KapoEp coimmunoprecipitated with secreted APP in conditioned media and full length APP in cell lysates (FIGS. 20A-20B). In addition, ApoEp and 6KApoEp colocalized with N-terminal APP in cultured CHO/APPwt cells as determined by immunofluorescence staining with 6H3B5 and pN-APP antibodies (FIG. 20C), respectively, confirming that N-terminal APP physically associates with apoE in cell culture. ApoE also coimmunoprecipated with N-terminal APP in homogenates prepared from brains of TgAPPwt mice, AD patients and age-match controls, confirming that the physical association of apoE with N-terminal APP occurs in vivo (FIGS. 20D-20E).

As in vitro confirmation of physical association of apoE with N-terminal APP, human recombinant sAPPβ (without Aβ domain), sAPPa, CHO/APPwt conditioned media-derived sAPPα (cmsAPPα) or APP N-terminal peptide (aa 31-60, APP31/60) was incubated with 6KapoEp, 6K, apoE3 or apoE4 followed by IP of apoE binding domain with 6H3B5 and analysis of sAPPα/β, APP31/60, 6KApoEp and apoE by WB (FIGS. 20F-20G). Human sAPPα was also incubated with apoE4 in the absence or presence of 6KApoEp or 6K followed by IP with pN-APP and analysis of apoE and sAPPα by WB (FIGS. 20H-20I). Overall, sAPPα/1 and APP31/60 coimmunoprecipitated with 6KApoEp and apoE in vitro, confirming that 6KApoEp and apoE physically associates with N-terminal APP, and 6KApoEp reduced this association.

6KApoEp Reduces A8 Production by Blocking apoE Interaction with N-Terminal Region of APP.

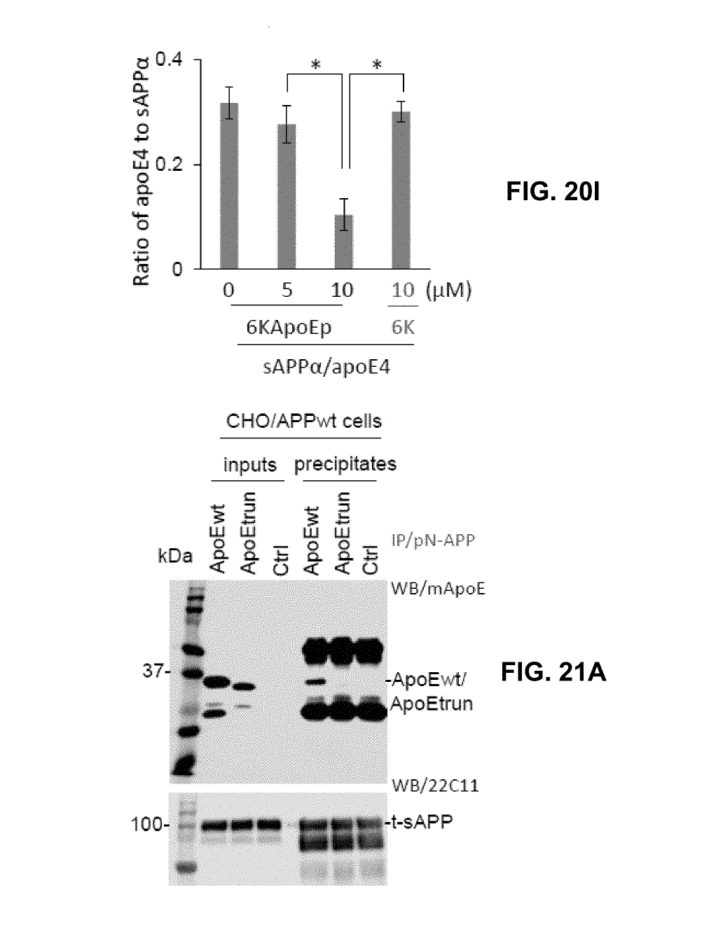

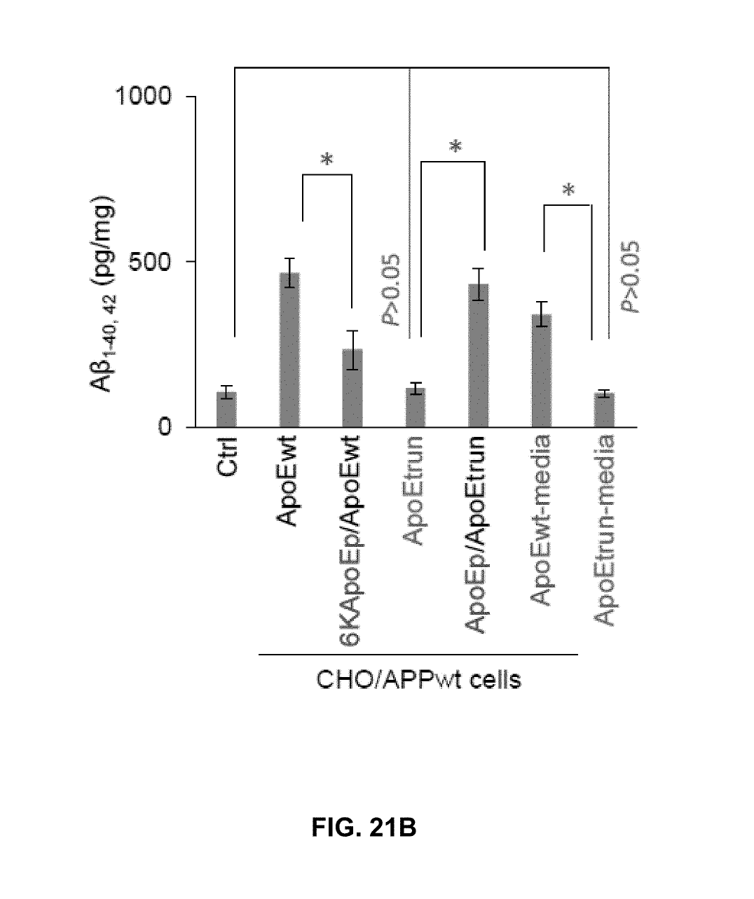

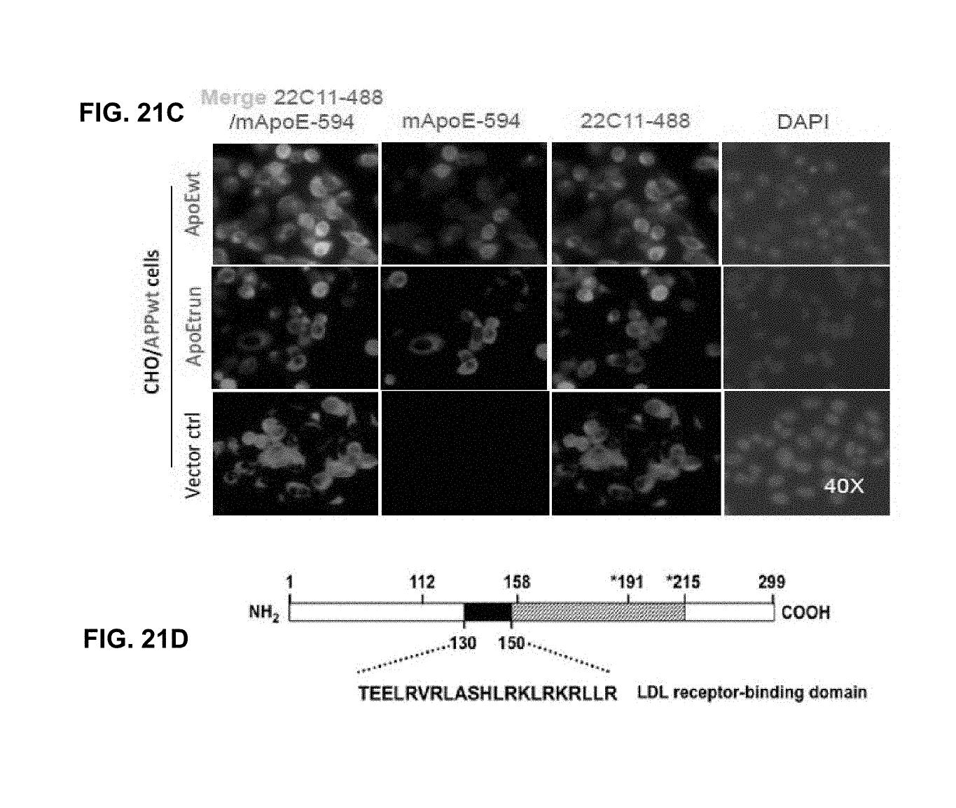

Since 6KApoEp reduced apoE and ApoEp-mediated Aβ production (FIGS. 19A-19C) as well as the physical association of apoE with N-terminal APP (FIGS. 20A-20I), we wished to determine if Aβ production is mediated by this interaction of apoE with N-terminal APP. Conditioned media collected from CHO/APPwt cells co-expressing human wild-type apoE4 (CHO/APPwt/ApoEwt cells), truncated apoE4 lacking the LDLR binding domain (CHO/APPwt/ApoEtrun cells) or control vector (CHO/APPwt/Ctrl cells) were IP with anti-N-terminal APP antibody (pN-APP) followed by analysis of apoE and sAPPα by WB. ApoEwt but not ApoEtrun coimmunoprecipitated with sAPPα (FIG. 21A). In addition, CHO/APPwt/ApoEwt cells produced markedly more Aβ than CHO/APPwt/ApoEtrun or CHO/APPwt/Ctrl cells (FIG. 21B). 6KApoEp reduced Aβ production in CHO/APPwt/ApoEwt cells and ApoEp enhanced Aβ production in CHO/APPwt/ApoEtrun cells, while conditioned media collected from CHO/APPwt/ApoEwt but not CHO/APPwt/ApoEtrun cells increased Aβ production in CHO/APPwt cells. Thus, the apoE binding domain is required for ApoE association with N-terminal APP and apoE-mediated Aβ production. As confirmation, ApoEwt but not ApoEtrun colocalized with N-terminal APP as determined by immunohistochemical staining (FIG. 21C).

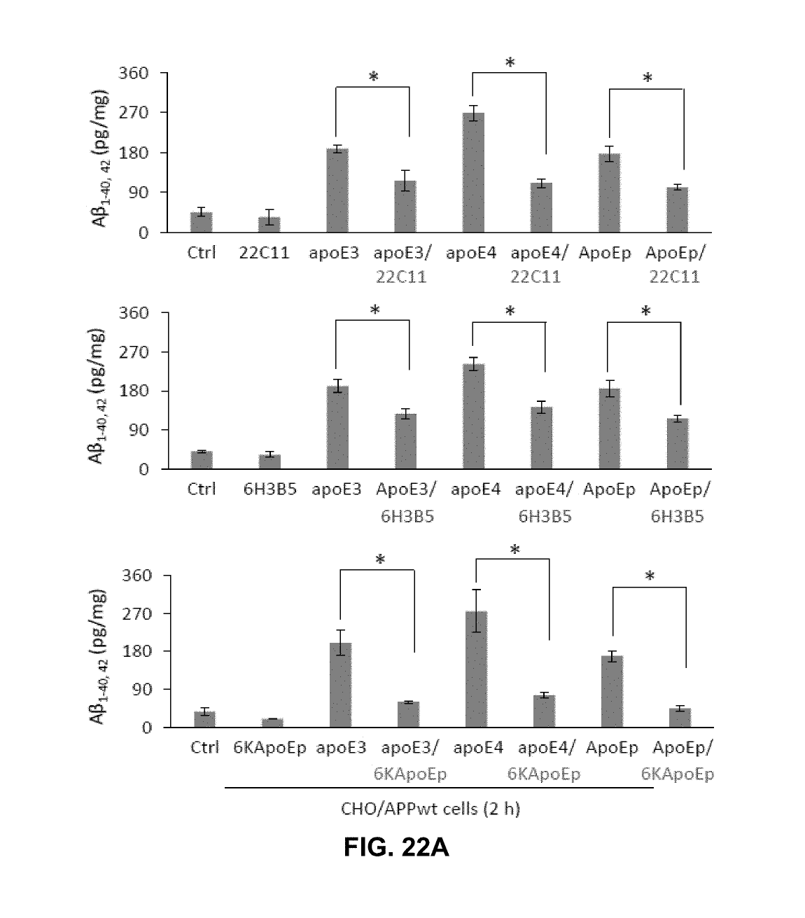

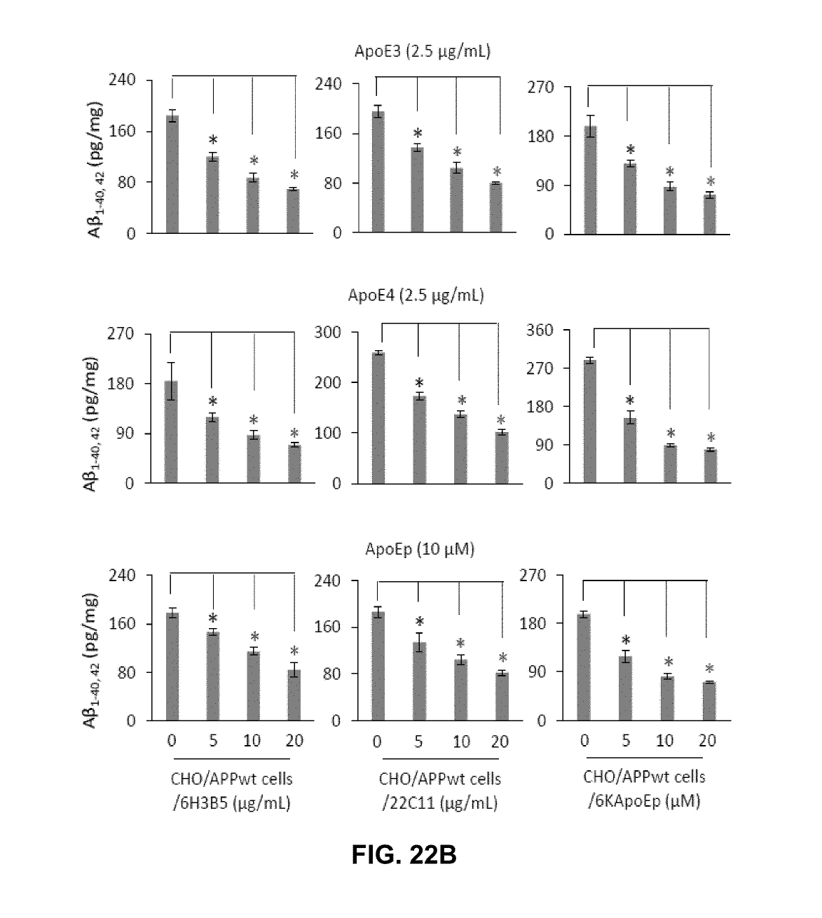

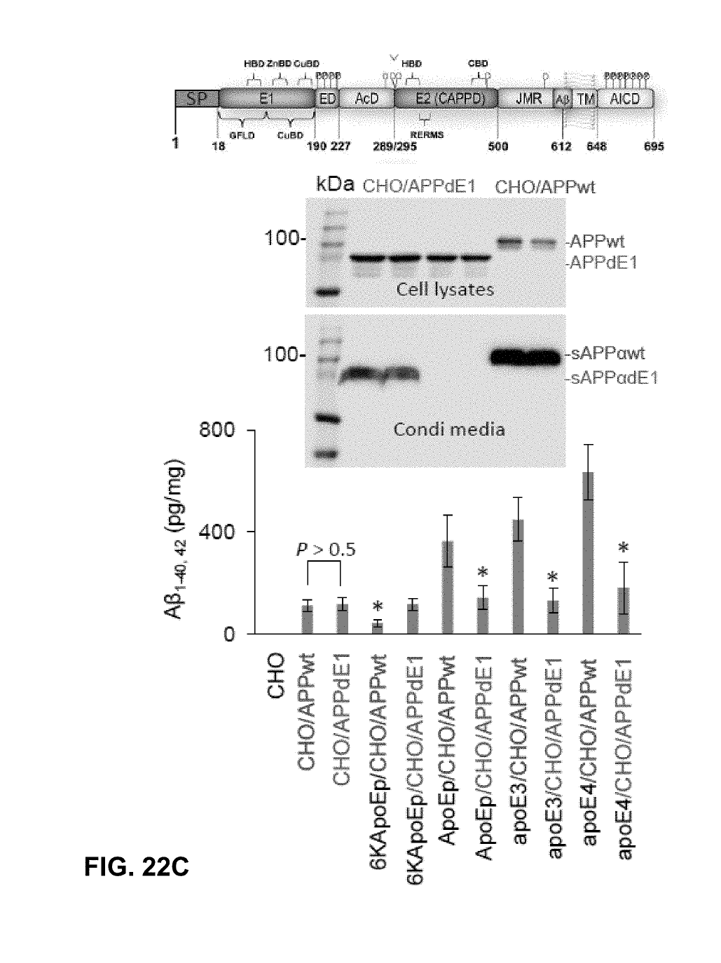

In order to confirm that apoE binding domain mediates Aβ production by interaction with the N-terminal region of APP, CHO/APPwt cells were treated with apoE3, apoE4 or apoEp in the absence or presence of N-terminal APP (22C11) or apoE binding domain antibodies (6H3B5) or 6KApoEp. Antibodies 22C11 and 6H3B5, and 6KApoEp, reduced apoE3, apoE4 and apoEp-mediated Aβ production in cultured cells in a dose-dependent fashion (FIGS. 22A-22B). In addition, CHO/APPwt cells and CHO cells expressing truncated APP lacking the N-terminal E1 region (CHO/APPdE1 cells) were treated with 6KApoEp, ApoEp, apoE3 or apoE4 followed by analysis of Aβ production. CHO/APPdE1 cells produced markedly less Aβ production compared with CHO/APPwt cells after treatment with ApoEp, apoE3 or apoE4 (FIG. 22C). In contrast, CHO/APPwt and CHO/APPdE1 cells elicited similar spontaneous Aβ production in the absence of apoE and 6KApoEp reduced Aβ production elicited by CHO/APPwt but not CHO/APPdE1 cells. Therefore, apoE and ApoEp mediates and 6KApoEp reduces Aβ production by binding to the N-terminal E1 region of APP, but this region of APP is not required for spontaneous Aβ release in the absence of apoE.

6KApoEp Inhibits Cell Surface APP Trafficking and p44/42 MAPK Phosphorylation.

Under normal physiological conditions, APP is known to be synthesized in the endoplasmic reticulum and trafficked via the trans-Golgi network to the plasma membrane where approximately 90% of it is cleaved by members of the ADAM family (α-secretase), yielding a membrane-bound α-carboxy-terminal APP fragment (α-CTF) and secreted sAPPα³⁷. The remaining unprocessed APP (about 10%) can be further trafficked back into the cell by endocytosis, followed by cleavage by β-site APP converting enzyme 1 (BACE1, known as β-secretase), yielding β-carboxy-terminal fragment (β-CTF) and sAPPβ, followed by γ-secretase processing ultimately generating A peptides which can then be secreted from the cell ³⁸⁻⁴⁰. Since α-secretase cuts APP within the Aβ region, increasing Aβ generation via the endocytic pathway precludes sAPPα production. Without being bound by theory, it was hypothesized that decreased Aβ generation by 6KApoEp might result from decreased membrane APP trafficking and subsequent amyloidogenic processing. To test this hypothesis, the effects of apoE4 and 6KApoEp on sAPPα and Aβ production, β-CTF levels and total APP levels in the plasma membrane was examined.

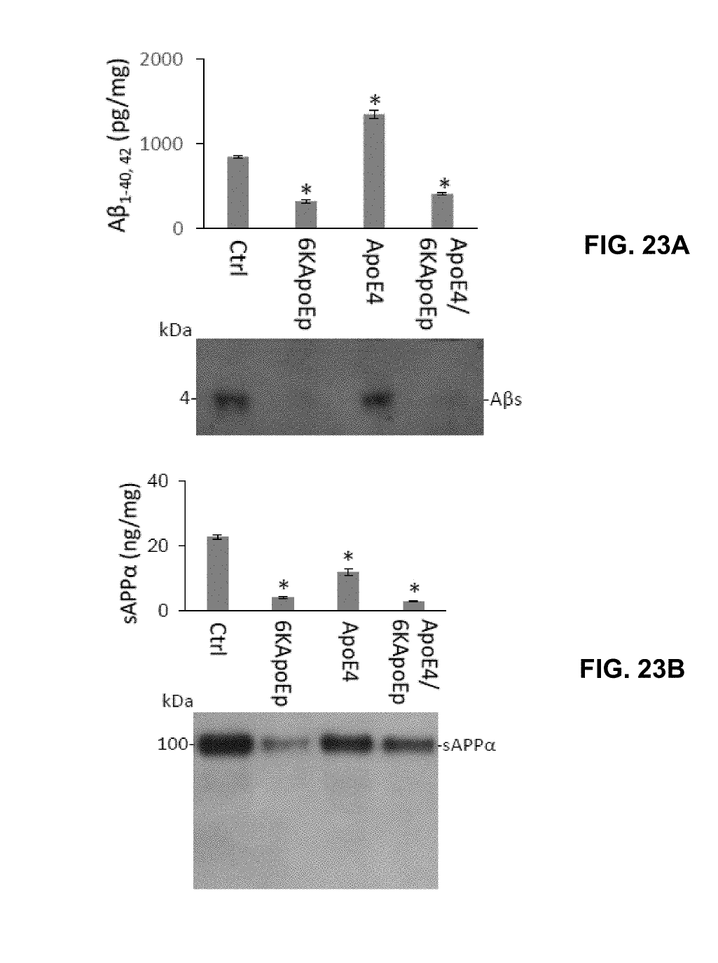

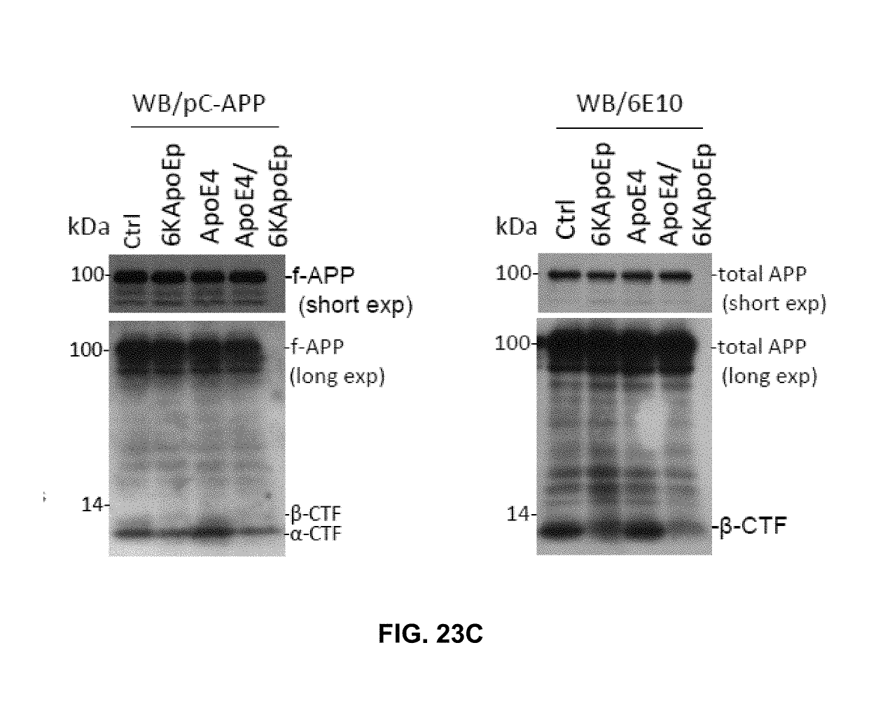

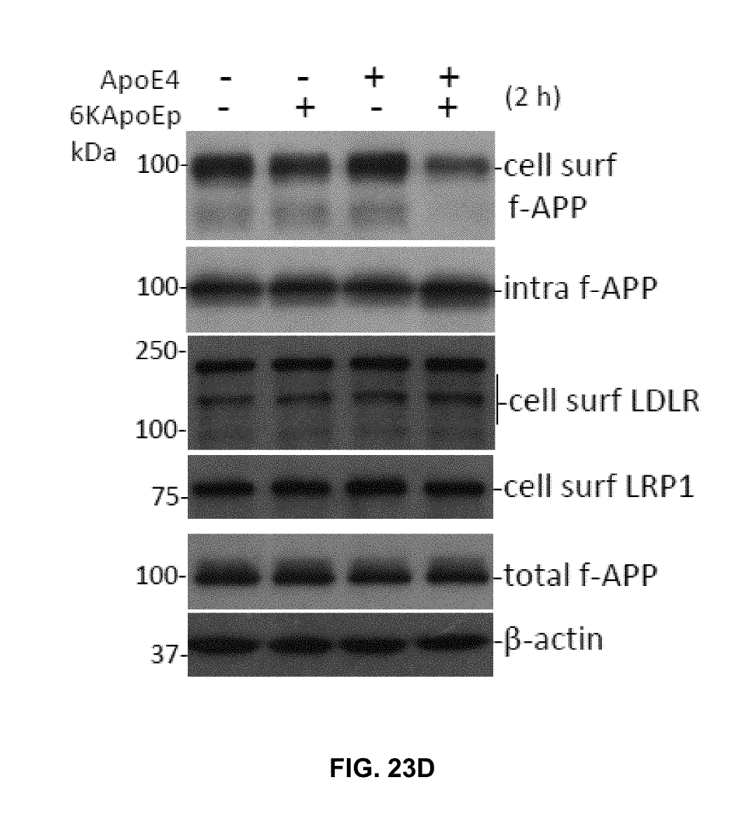

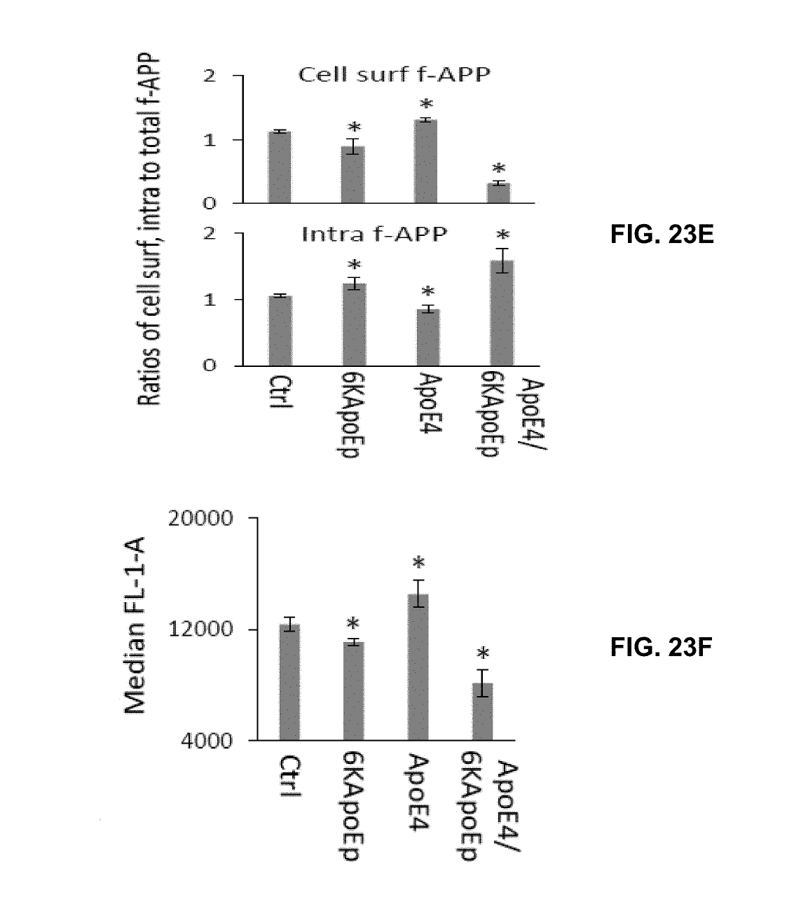

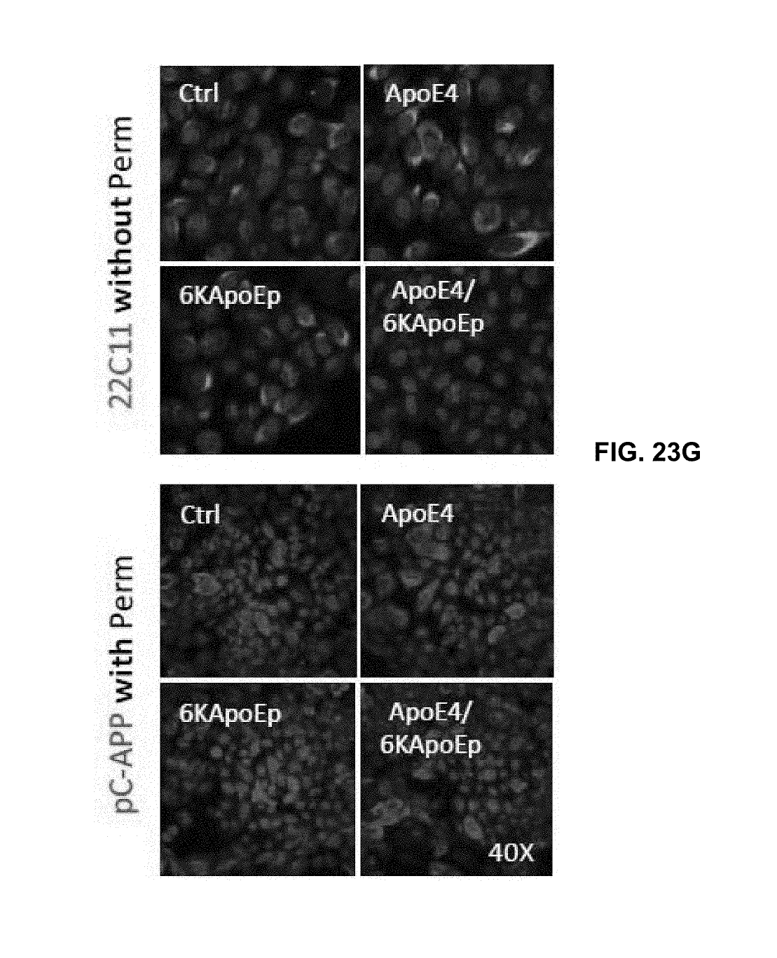

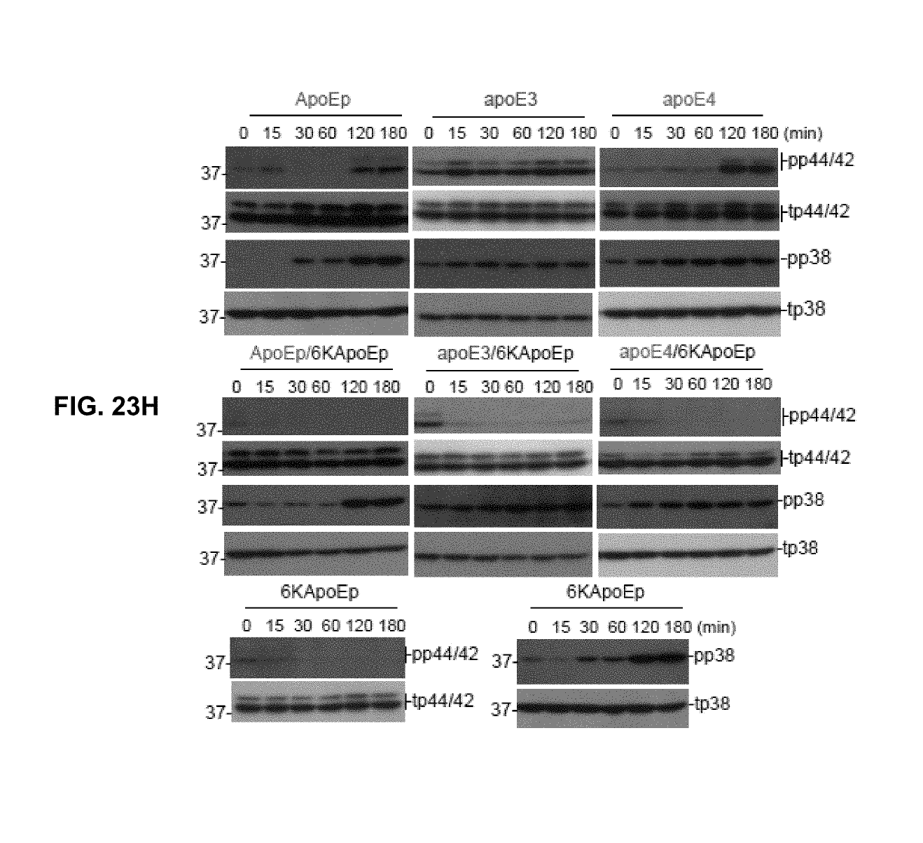

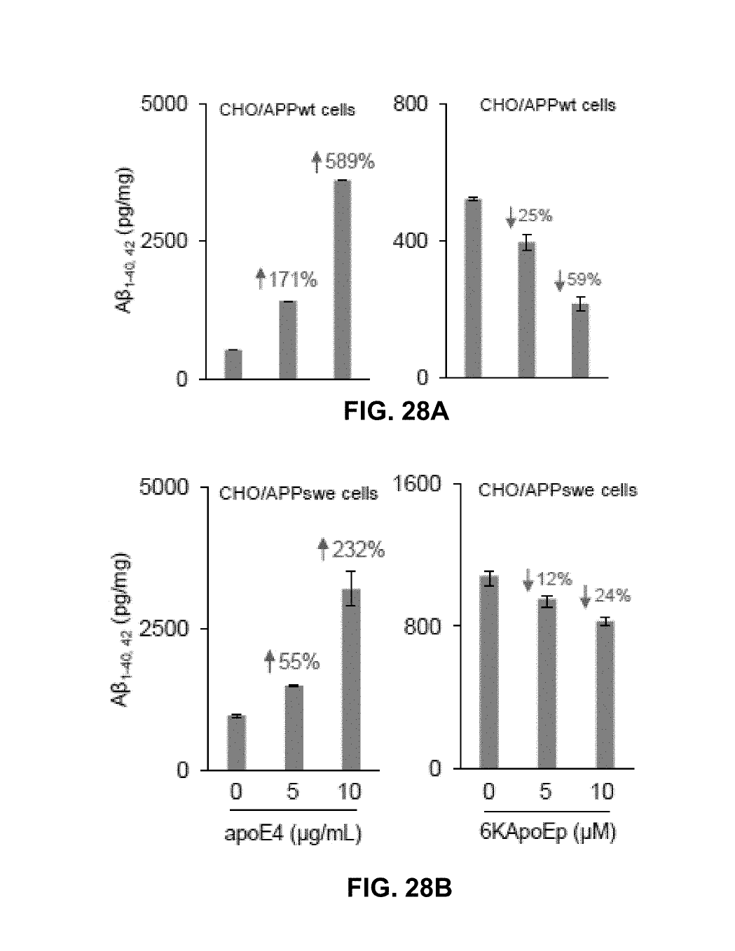

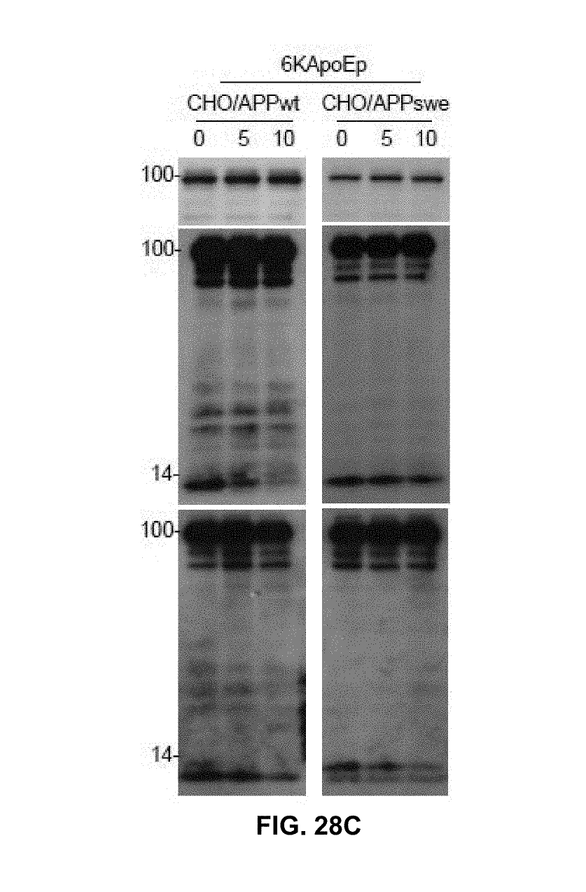

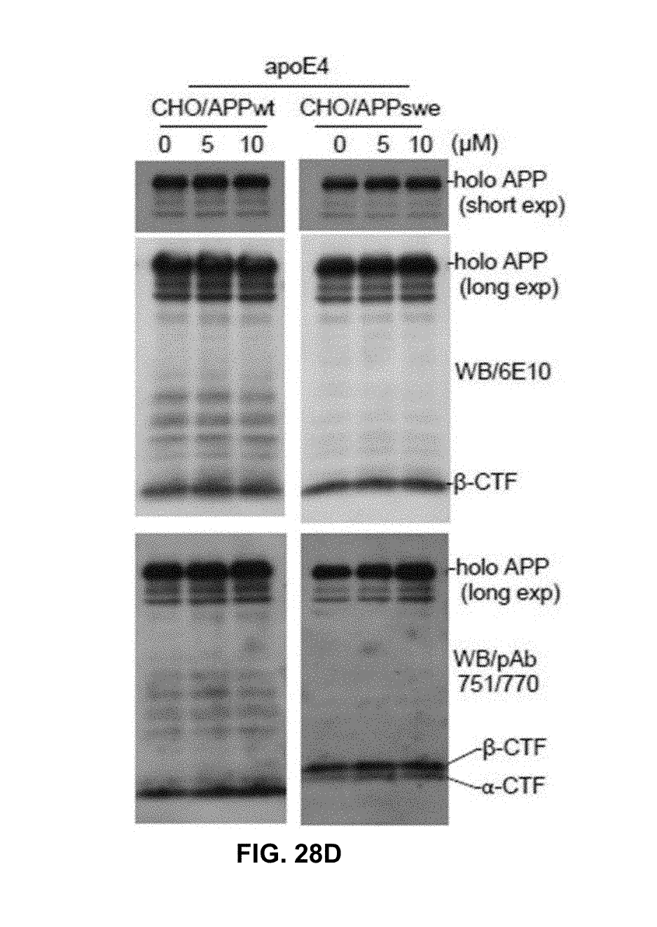

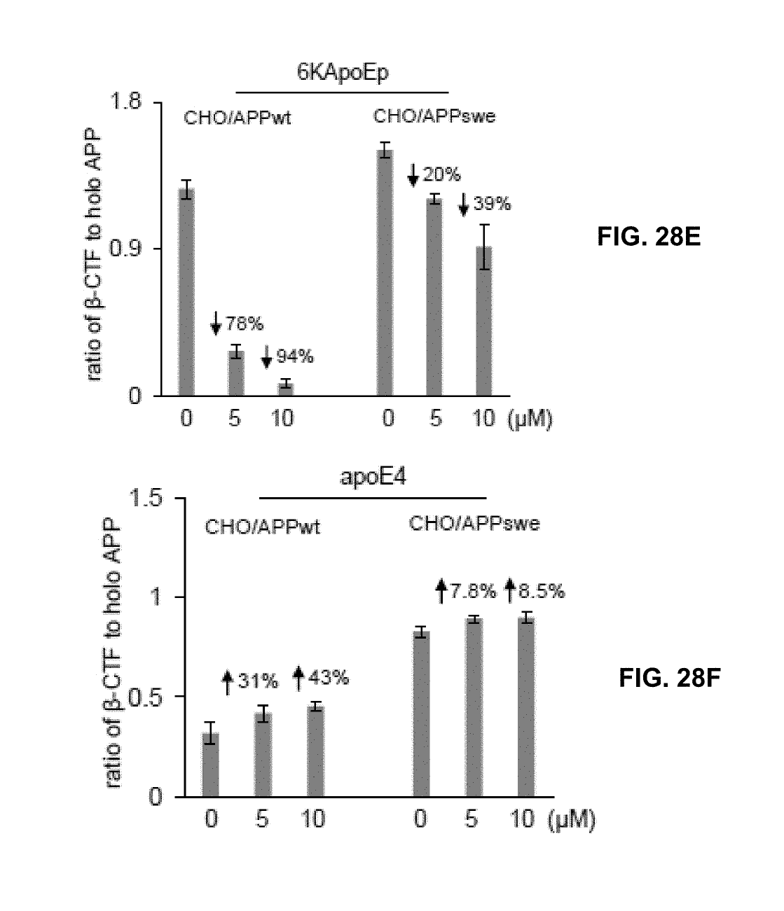

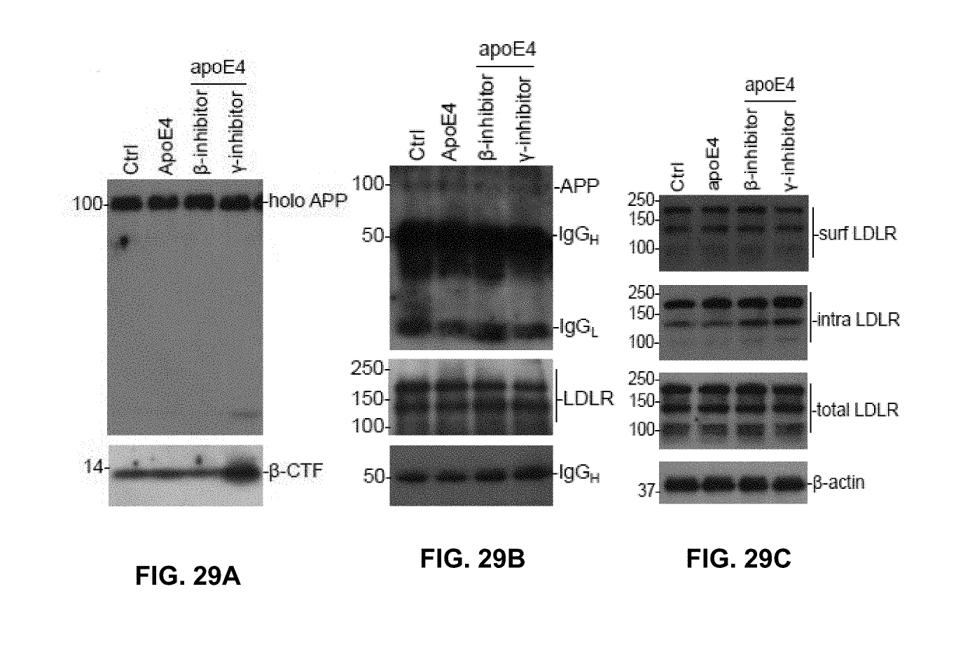

Treatment of CHO/APPwt cells with apoE4 markedly increased Aβ and β-CTF levels, while reducing sAPPα levels, presumably by enhancing APP endocytosis (FIGS. 23A-23H). Indeed, apoE4 enhanced Aβ and β-CTF levels much more in CHO/APPwt cells compared with CHO cells overexpressing APP with the Swedish mutation (CHO/APPswe cells), which is a better substrate for BACE1 and directly processed to Aβ prior to its trafficking to the cell surface (FIGS. 28A-28F). In contrast, 6KApoEp reduced both basal and more profoundly apoE4-induced production of Aβ, β-CTF and sAPPα, without altering total APP levels, suggesting that 6KApoEp inhibits an earlystage of APP processing, such as the initial trafficking of APP to the plasma membrane. As further confirmation, 6KApoEp reduced both basal and apoE4-mediated cell surface APP levels, as determined by WB, flow cytometry and confocal microscopy, regardless of whether the cells were pre-treated with 6KApoEp, co-treated with 6KApoEp together with apoE4, or treated with apoE4 pre-incubated with 6KApoEp (FIGS. 23D-23G and FIGS. 27A-27B). 6KApoEp also reduced Aβ and β-CTF levels more in CHO/APPwt than in CHO/APPswe cells, consistent with reduction of APP trafficking to the cell surface (FIGS. 28A-28F). Notably, apoE4 and 6KApoEp, in the absence or presence of β- or γ-secretase inhibitors, did not alter cell surface protein levels of LDLR or LRP1 or mRNA levels of APP or LDLR, suggesting that ApoE and 6KApoEp do not alter APP, LDLR and LRP1 expression or recycling (FIG. 23D; FIGS. 29A-29C).

In a previous study, apoE-mediated APP transcription/translation and Aβ production were found to be mediated by activation of a non-canonical p44/42 mitogen-activated protein kinase (MAPK)³³. In order to determine if apoE and ApoEp-mediated APP trafficking and processing might also be mediated by this signaling pathway, the effects of apoE, ApoEp and 6KapoEp on p44/42 and p38 MAPK phosphorylation was determined. While ApoEp, apoE3 and apoE4 activated both p44/42 and p38 MAPK phosphorylation, 6KApoEp only activated p38 MAPK phosphorylation and only inhibited ApoE-induced p44/42 phosphorylation (FIG. 23H). Taken together and without being bound by theory, these findings collectively suggest that ApoEp, ApoE4 and 6KApoEp may have different effects on APP processing, with ApoEp and ApoE4 enhancing and 6KApoEp reducing cell surface APP trafficking, APP endocytosis and amyloidogenic processing, potentially mediated by differential activation of MAPK pathways.

6KApoEp Reduces Cerebral β-Amyloid and Tau Pathologies as Well as Memory Impairment in Alzheimer Model Mice.

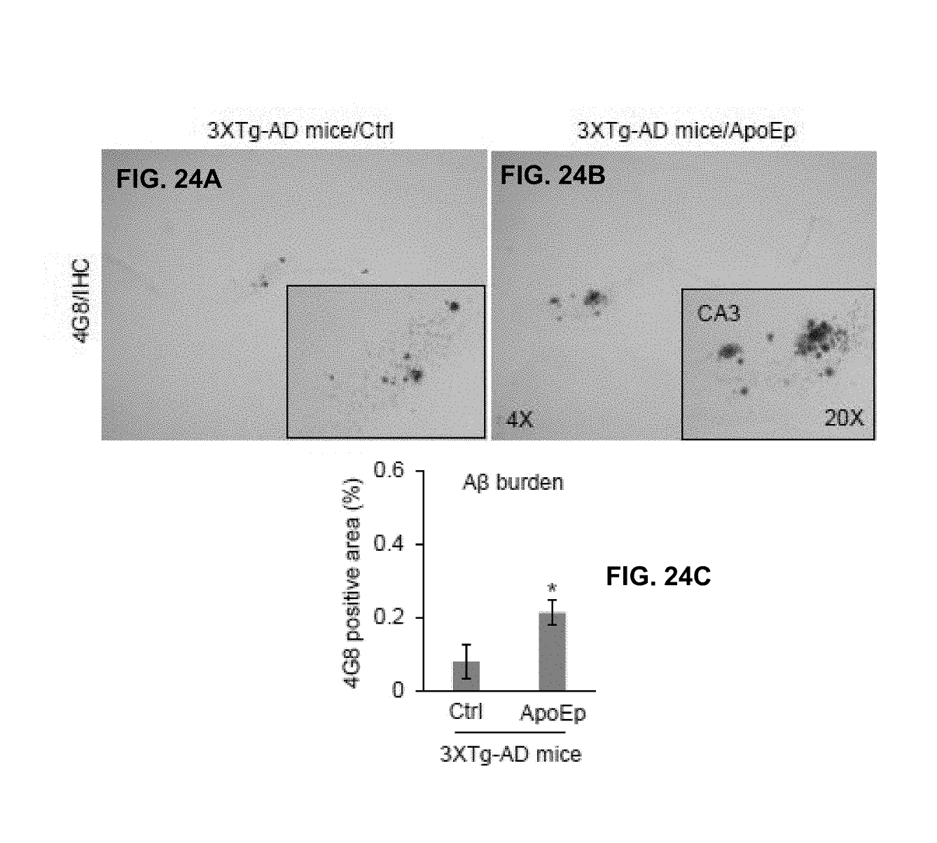

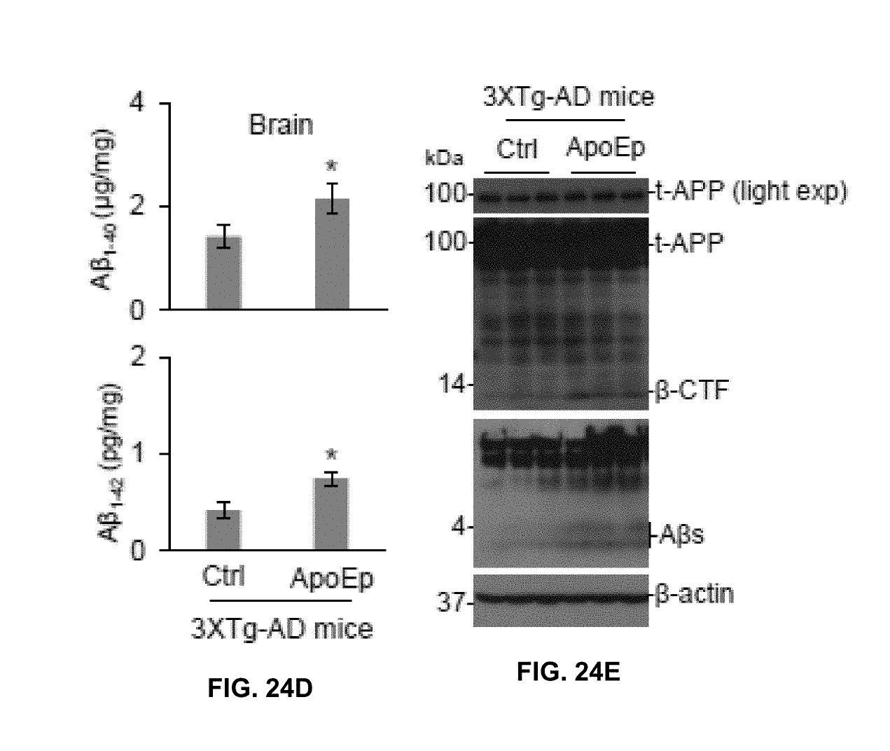

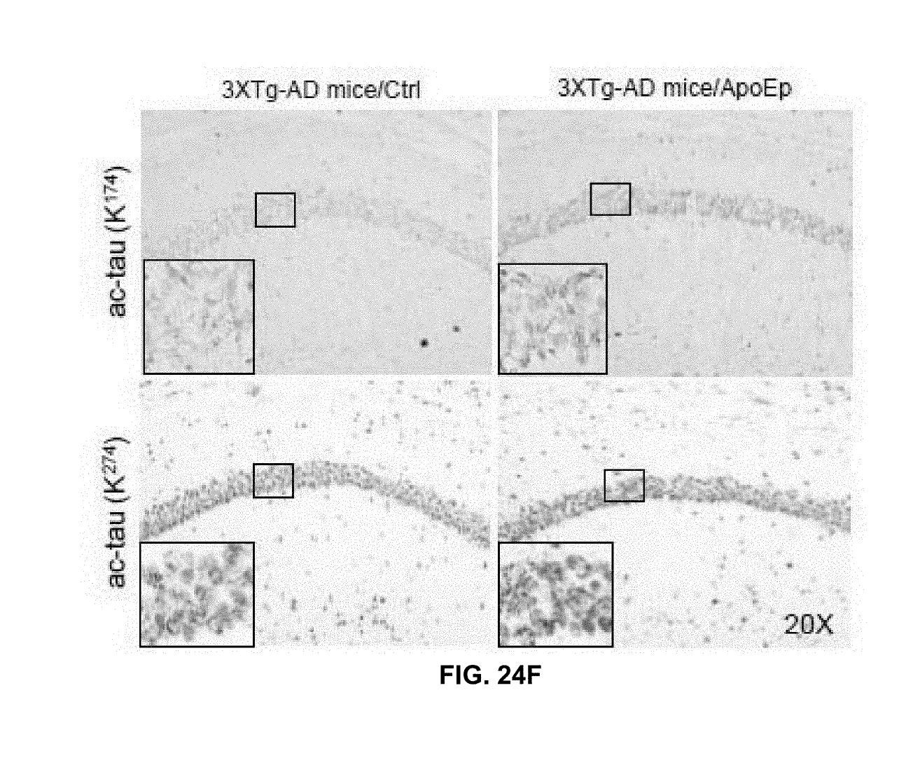

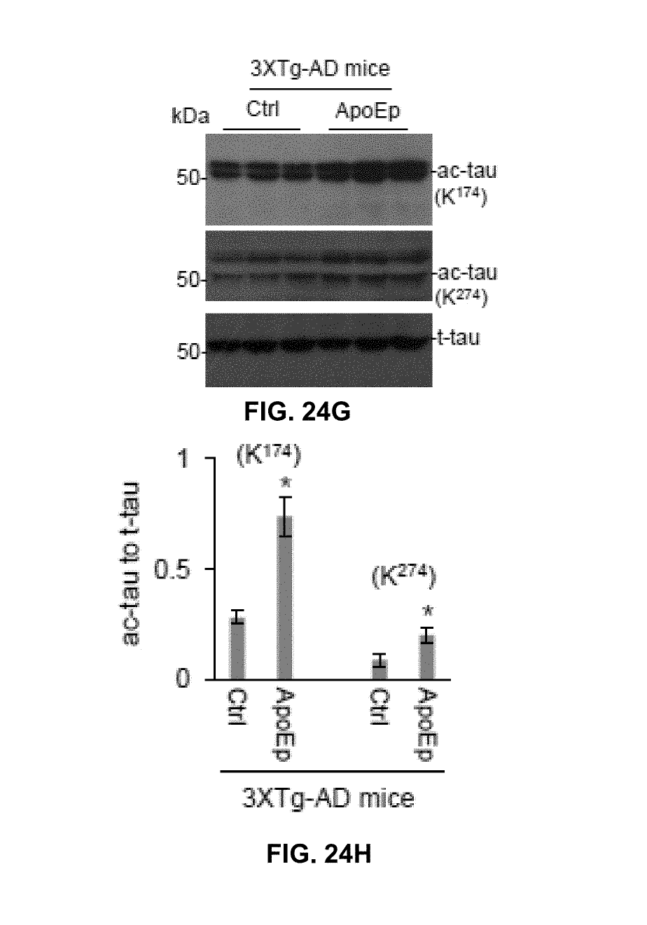

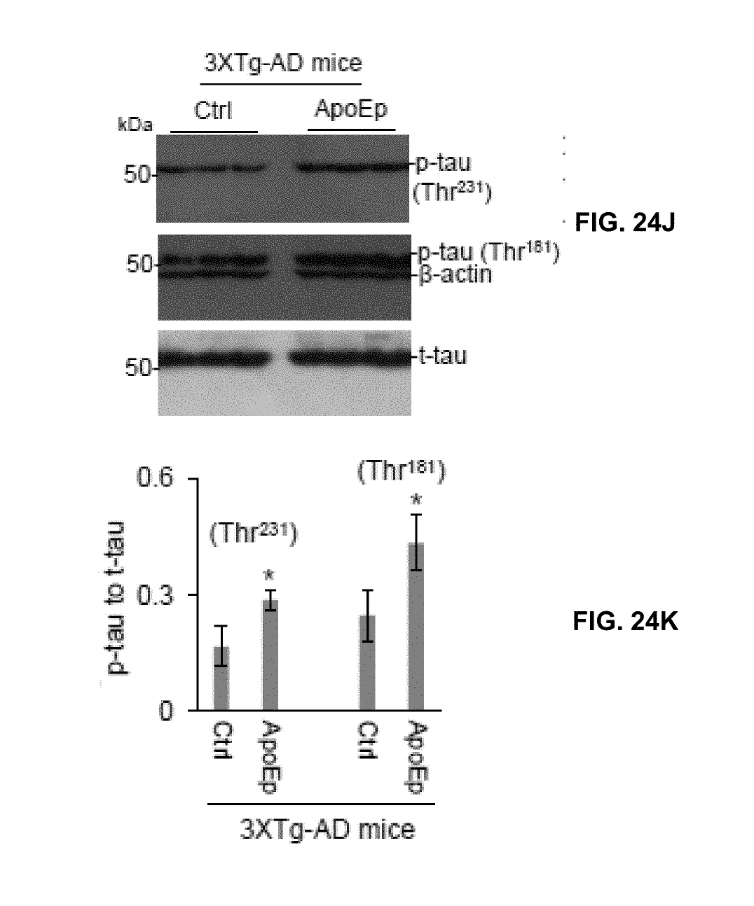

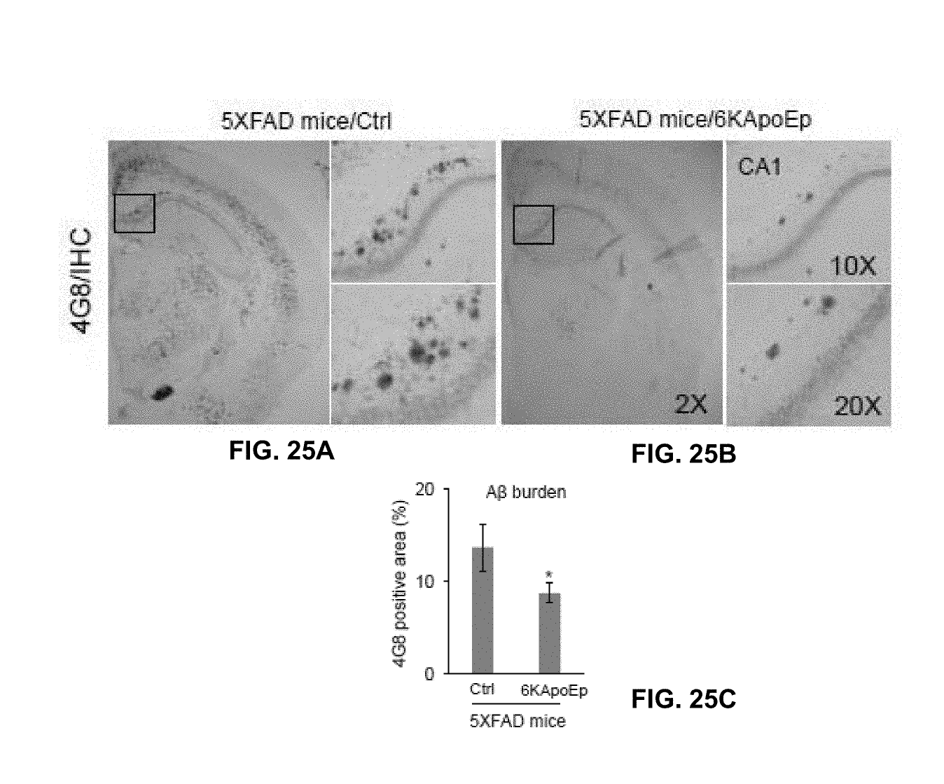

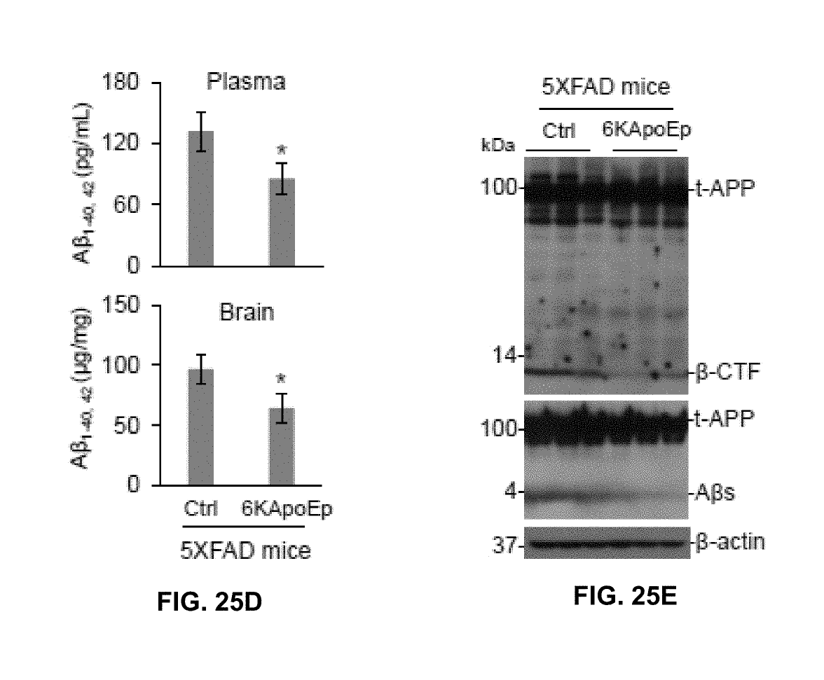

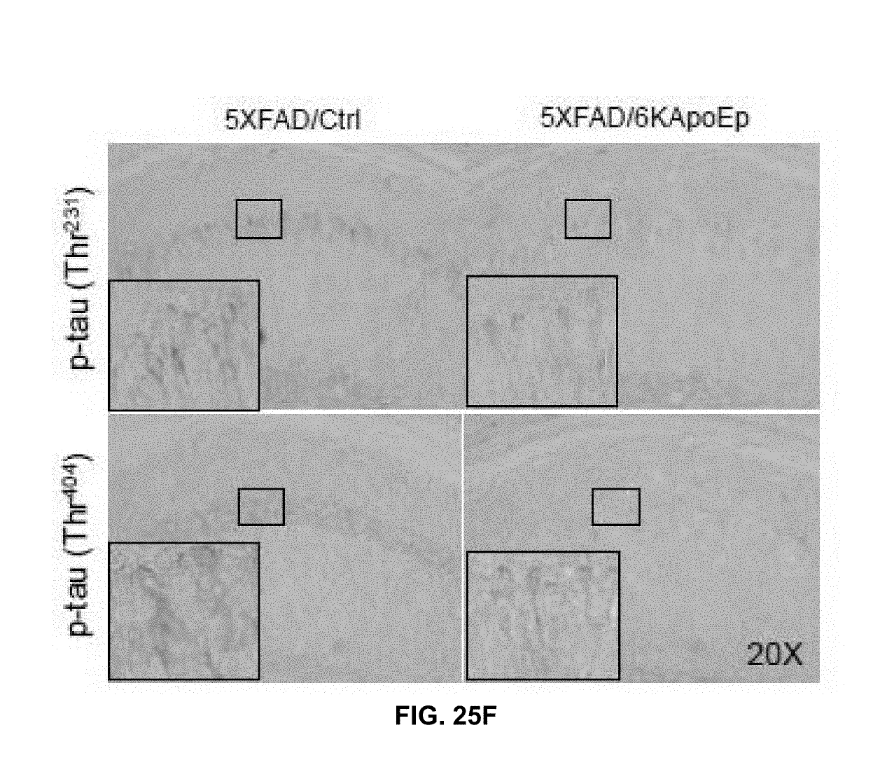

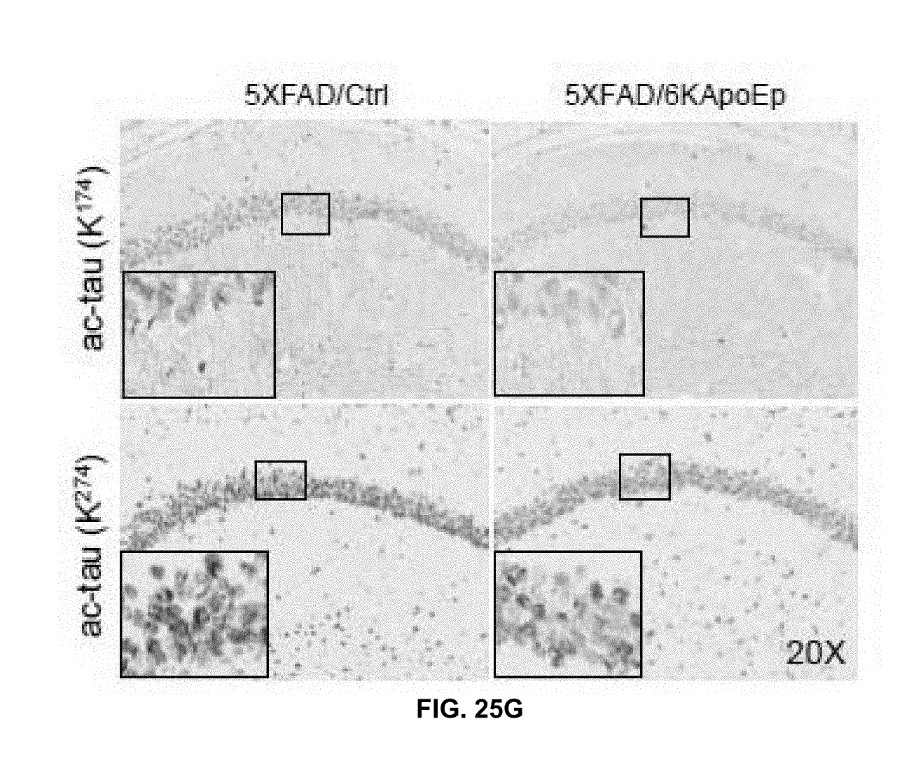

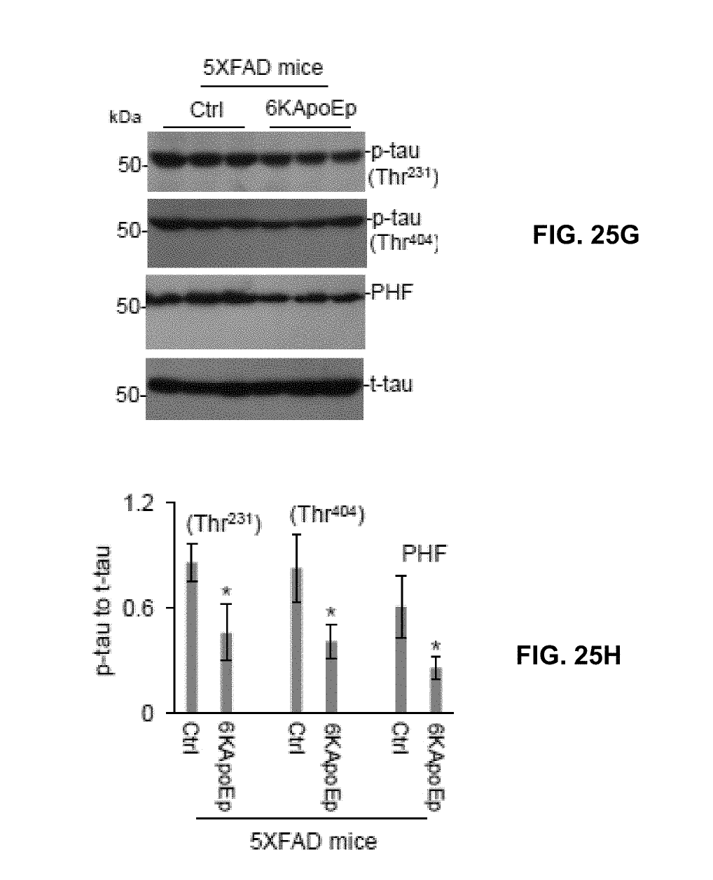

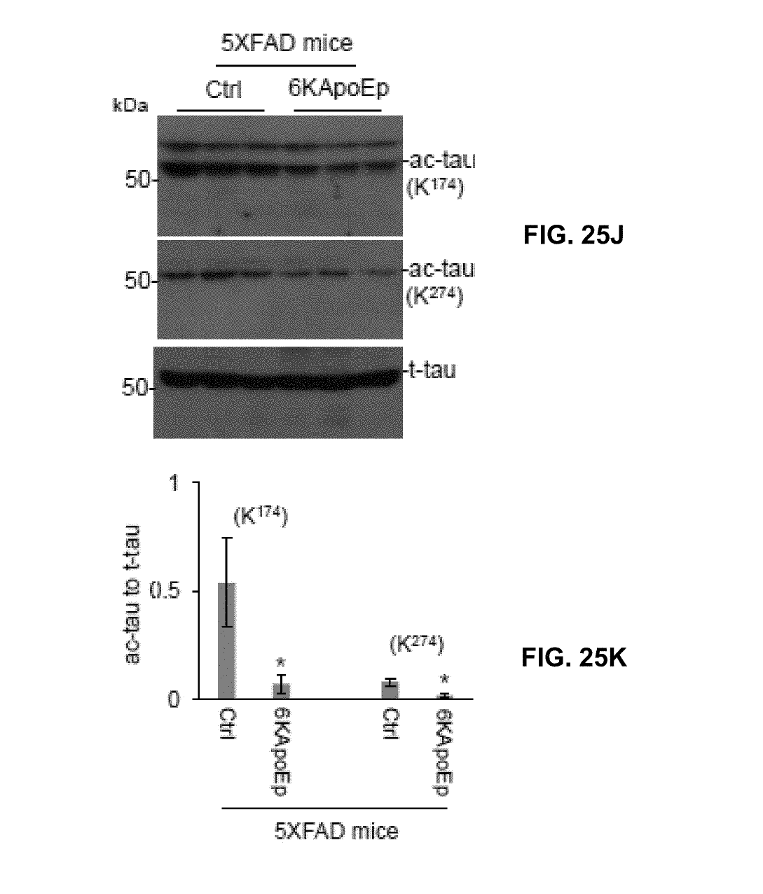

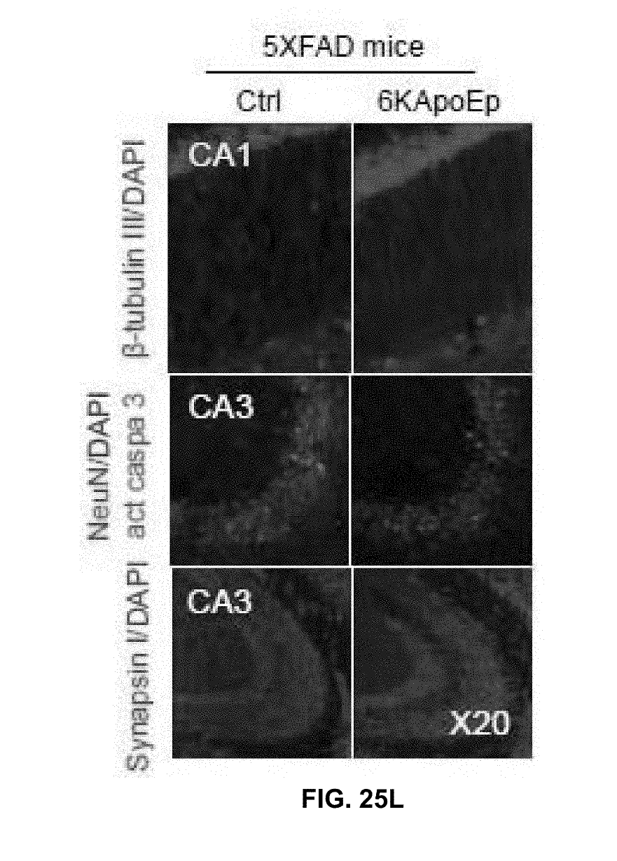

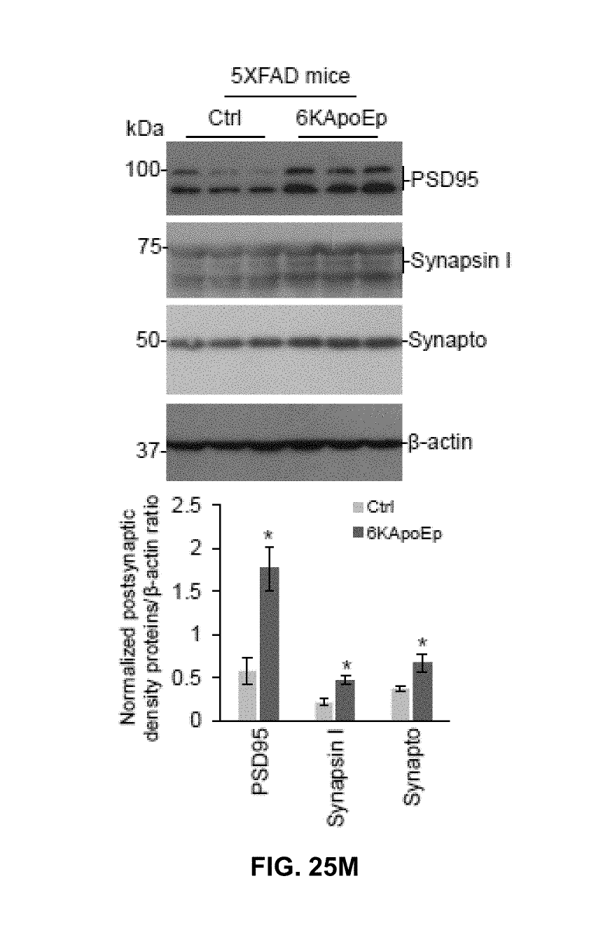

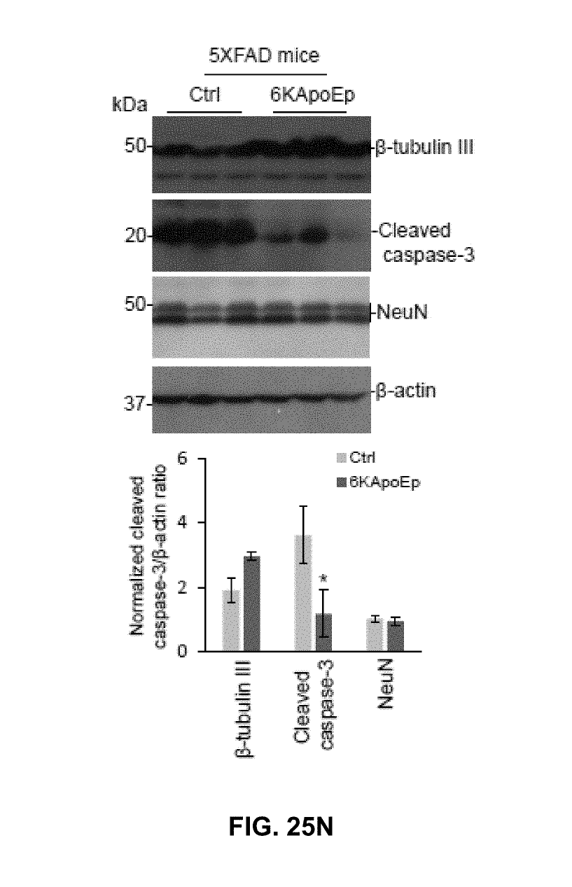

Since 6KApoEp inhibit ApoE-APP receptor mediated Aβ generation, it was examined if this apoE antagonist could reduce AD-like pathology in 5×FAD mice, known to develop extensive and aggressive amyloid pathology in the brain. In addition, it was examined if mimicking the function of apoE lipoproteins by treatment with ApoEp could accelerate AD-like pathologies in 3×Tg-AD mice, where Aβ seeding might play role in accelerating the progression of tau neuropathology. These mice were treated with ApoEp or 6KApoEp by intraperitoneal injection for 12 weeks and then euthanized, followed by analysis of cerebral Aβ and tau pathology. Peripheral treatment of 3×Tg-AD mice with ApoEp increased Aβ plaques, as visualized by immunohistochemistry (IHC) with antibody 4G8, in comparison with mice treated with PBS as control (FIGS. 24A-24C). Correspondingly, ApoEp increased levels of soluble Aβ1-40, 42 and β-CTF, as determined by ELISA and WB (FIGS. 24D-24E), as well as levels of acetylated and phosphorylated tau, as evidenced by IHC and WB (FIGS. 24F-24J). In contrast, 6KApoEp treatment reduced Aβ plaques, Aβ and β-CTF levels (FIGS. 25A-25E) as well as phosphorylated and acetylated tau in 5×FAD mice (FIGS. 25F-25K). In addition, 6KApoEp treatment reduced neuronal apoptosis (as determined by levels of cleaved caspase 3), while enhancing neurogenesis (synapsin 1) and synaptogenesis (presynaptic synaptophysin and postsynaptic PSD95; FIGS. 25L-25N). Notably, peripheral treatment with both biotin-ApoEp and biotin-6KApoEp increased levels of these peptides in the brain within 30 min of treatment, as determined by ELISA (data not shown).

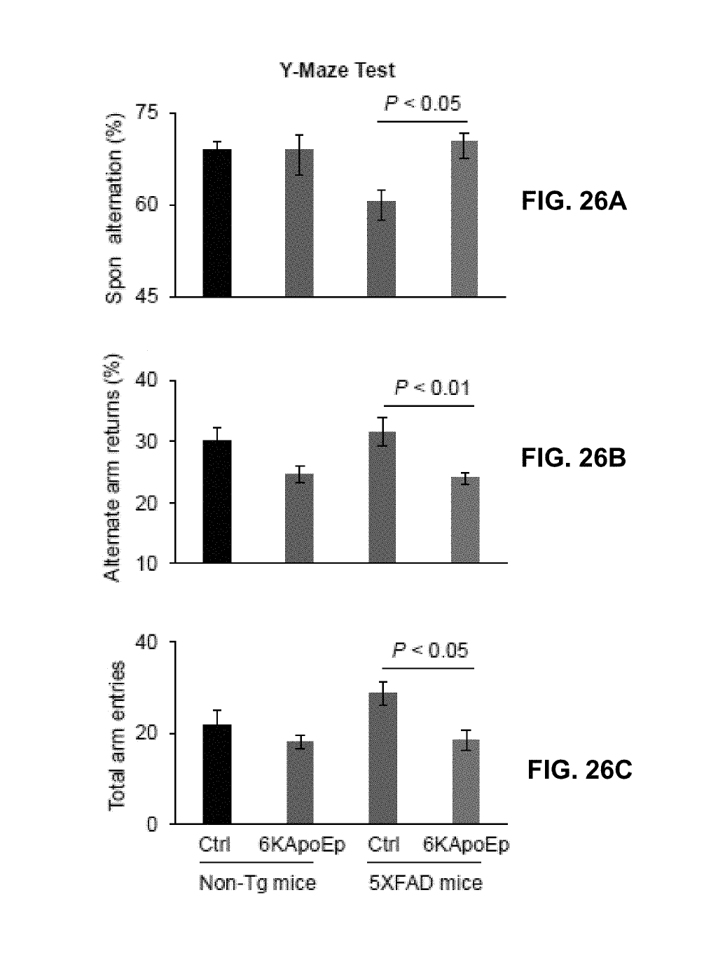

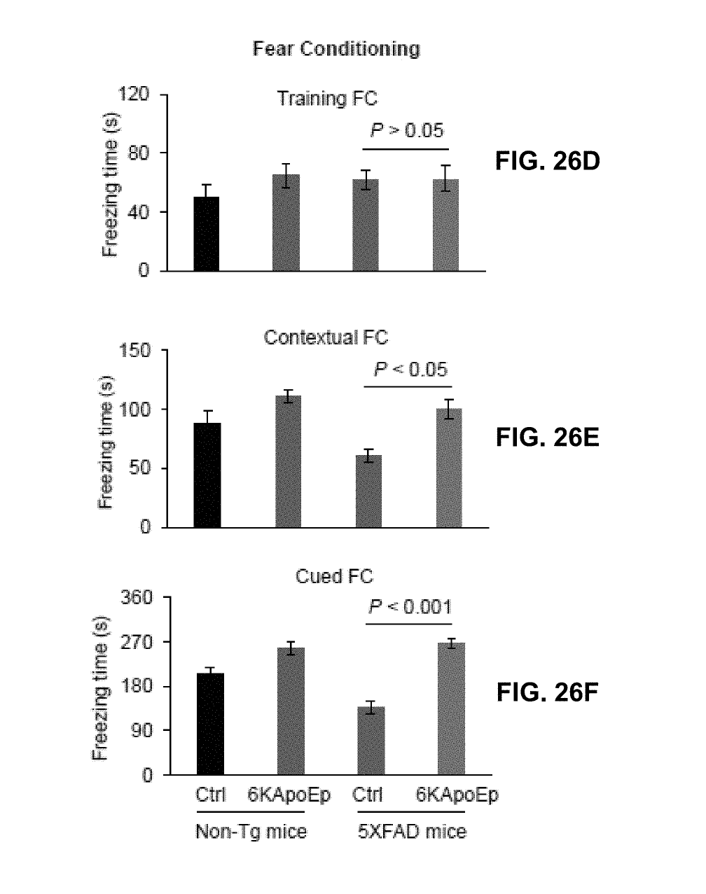

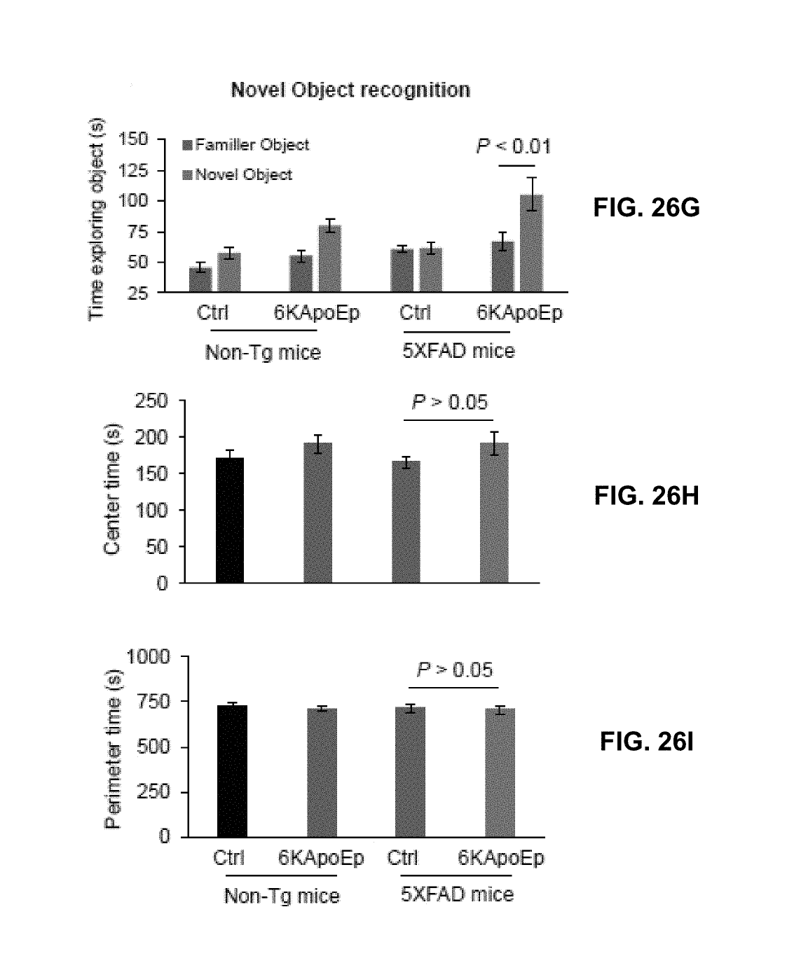

In addition to determination of Aβ and tau pathologies, the effect of 6KApoEp on AD-like hippocampus-dependent learning and memory impairment were determined in 5×FAD mice utilizing Y maze, fear conditioning and novel object recognition tests. Untreated 5×FAD mice exhibited learning and memory impairment compared with nontransgenic control mice as determined by reduced spontaneous alternation in the Y maze, which was reversed by 6KApoEp treatment (FIG. 26A). 6KApoEp treatment also reduced alternate arm entry returns in both 5×FAD and nontransgenic control mice (FIG. 26B). Likewise, learning impairment in 5×FAD mice was exhibited by reduced freezing times during contextual and cued testing after fear conditioning (FIG. 26E-26F) and reduced ability to discriminate between familiar and novel objects (FIG. 26G), which were all reversed upon treatment with 6KApoEp. Notably, 5×FAD mice exhibited some hyperactivity, as shown by enhanced total arm entries in the Y maze, which was reversed by 6KApoEp treatment (FIG. 26C). However, 5×FAD and non-transgenic mice, whether untreated or treated with 6KApoEp, exhibited similar levels of anxiety since they spent similar amounts of time in central and peripheral zones of the open field (FIGS. 26H-26I). Overall, these results confirm that while apoE accelerates Aβ and tau pathologies, antagonizing the effect of apoE by 6KApoEp reduces AD pathology, learning impairment and hyperactivity in an AD mouse model.

### Discussion

In this Example, the interaction of N-terminal region of APP with apoE as a therapeutic target for AD was examined. Based on previous studies which suggest that apoE enhances Aβ production by binding to the N-terminal region of APP^{32,33}, we initially focused on the apoE LDLR binding domain (residues 133-152, ApoEp). Like apoE, it was found that ApoEp also dose-dependently increased Aβ production in CHO/APPwt cells, showing that the apoE binding domain is responsible for this action (FIGS. 19A-19C). It was found that while structural modification of ApoEp by amino terminal addition of Flag enhanced the potency of ApoEp in mediating Aβ production, amino terminal addition of lysine residues converted ApoEp from a promoter to inhibitor. It was found 6KApoEp to be an effective antagonist against Aβ production mediated by lipidated and natural apoE in cultures of human neuroblastoma cells and neurons. It was confirmed by immunoprecipitation analysis that apoE directly binds to the N-terminal region of APP and that 6KApoEp interferes with this interaction (FIGS. 20A-20I). It was also observed that endogenous apoE also binds N-terminal APP in homogenates of APPwt mice, AD patients and age-matched controls, indicating that the interaction of apoE with N-terminal APP occurs in vivo. In addition, antibodies against the N-terminal region of APP (22C11) and the apoE binding domain (6H3B5), as well as N-terminal truncation of APP, reduced Aβ production elicited by apoE and ApoEp (FIGS. 21A-22C).Overall, these studies indicate that blocking apoE interaction with N-terminal APP reduces Aβ production and can be a viable strategy for the development of a AD therapeutic.

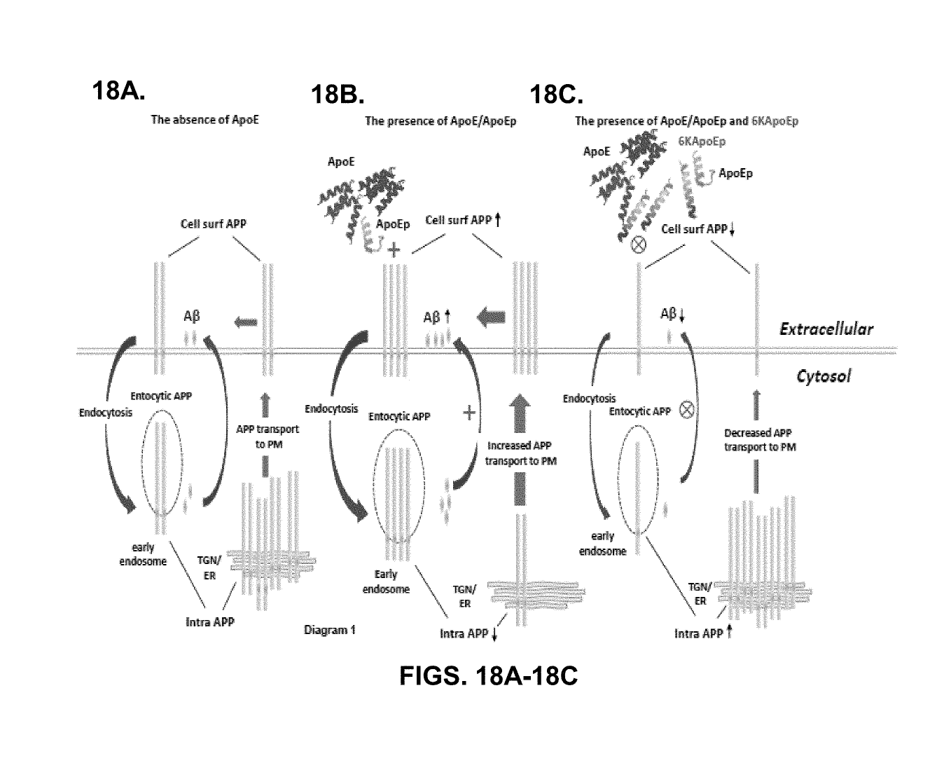

The mechanism by which 6KApoEp reduces apoE4-induced Aβ production appears to primarily involve inhibiting the early steps of APP trafficking to the cell membrane (FIGS. 18A-18C). While the ApoE mimetic peptide (ApoEp) enhanced Aβ and reduced sAPPo production, likely by enhancing APP endocytosis and subsequent amyloidogenic proteolysis, 6KApoEp reduced Aβ, β-CTF and sAPPo production, presumably by inhibiting early APP trafficking without altering total APP levels (FIGS. 23A-23H). This is supported by the observation that 6KApoEp reduced cell surface levels of APP, as determined by WB, flow cytometry and confocal microscopy. In addition, 6KApoEp reduced Aβ production and β-CTF levels more in CHO/APPwt cells, where A□production is mediated primarily by APP endocytosis and subsequent proteolysis, than in CHO/APPswe cells, where Aβ production occurs prior to APP trafficking to the cell surface as a consequence of APPswe being a better substrate for BACE1 (FIGS. 28A-28F). Therefore, 6KApoEp may be a therapeutic for treatment of SAD patients who express wild-type APP rather than for patients with familial AD who express APP mutations. ApoEp also enhanced Aβ production more in CHO/APPwt cells than in CHO/APPswe cells, presumably because ApoEp enhances APPwt trafficking and endocytosis, while having less effect on Aβ production from APPswe. Previously, Huang et al. (2017) found that apoE enhances Aβ production in human neurons by activating a non-canonical dual leucine-zipper kinase, MAP-kinase kinase (MAPKK), which activates p44/42 MAP kinase (Erk1/2) phosphorylation, cFos phosphorylation, transcription factor Aβ-1 and APP transcription/translation³³. These results support that apoE activates p44/42 MAPK phosphorylation, found that both apoE and 6KApoEp also activates p38 MAPK phosphorylation and that 6KApoEp reduces apoE-mediated p42/44 MAPK phosphorylation (FIG. 23H). Clearly apoE and 6KApoEp have different effects on APP trafficking and processing, which may be mediated in part by activating different MAPK pathways.

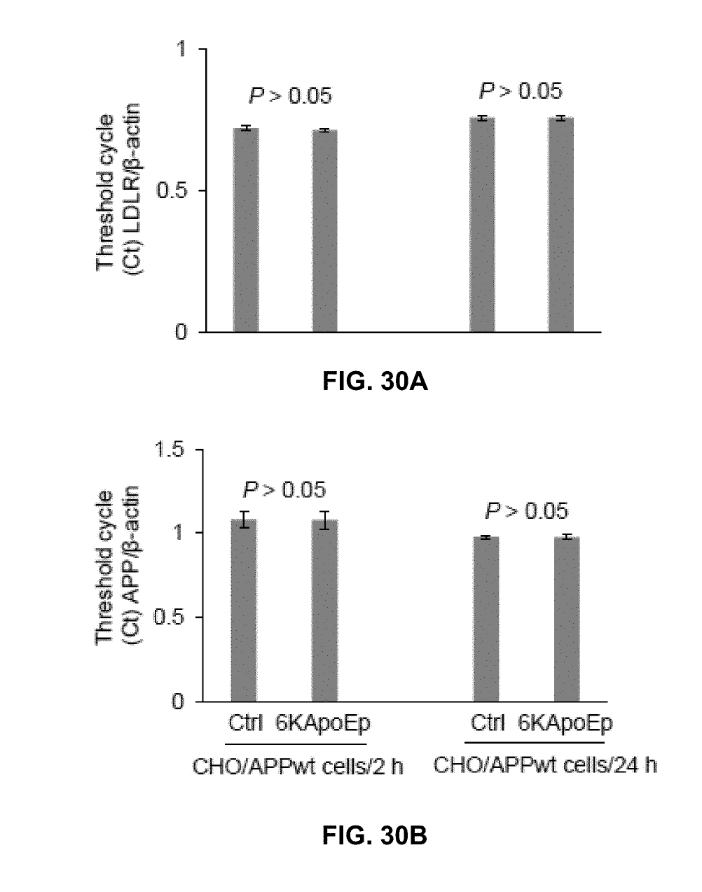

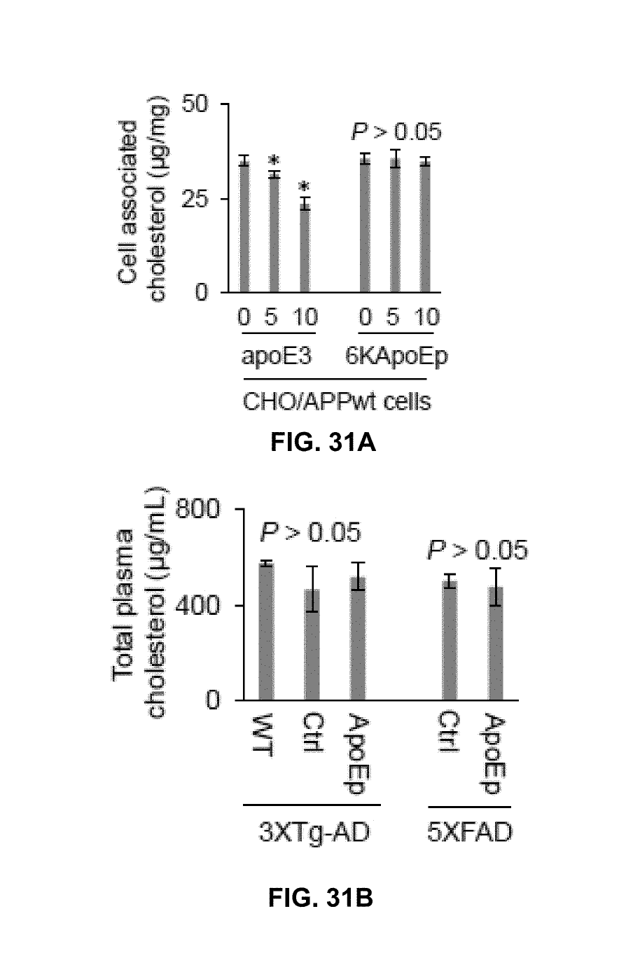

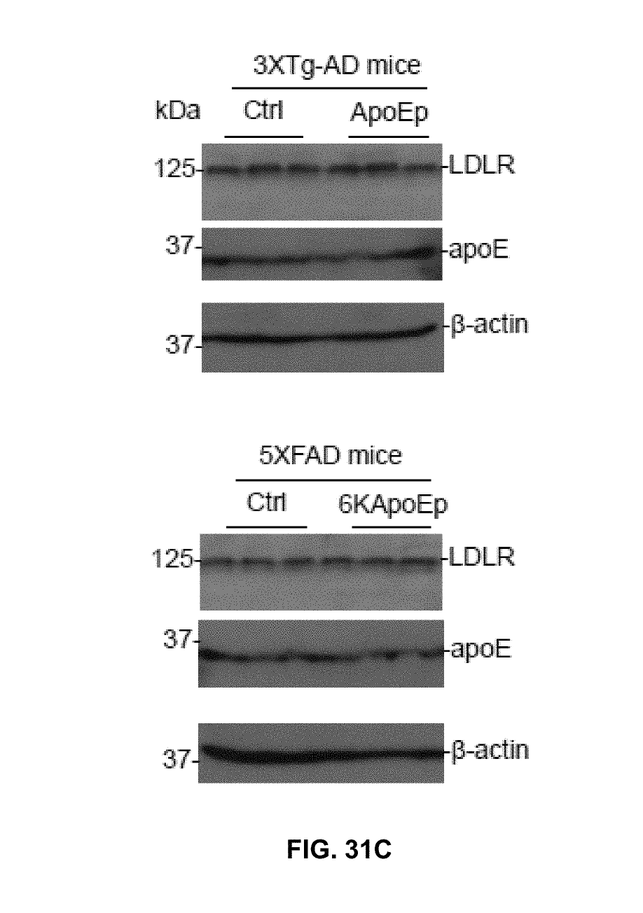

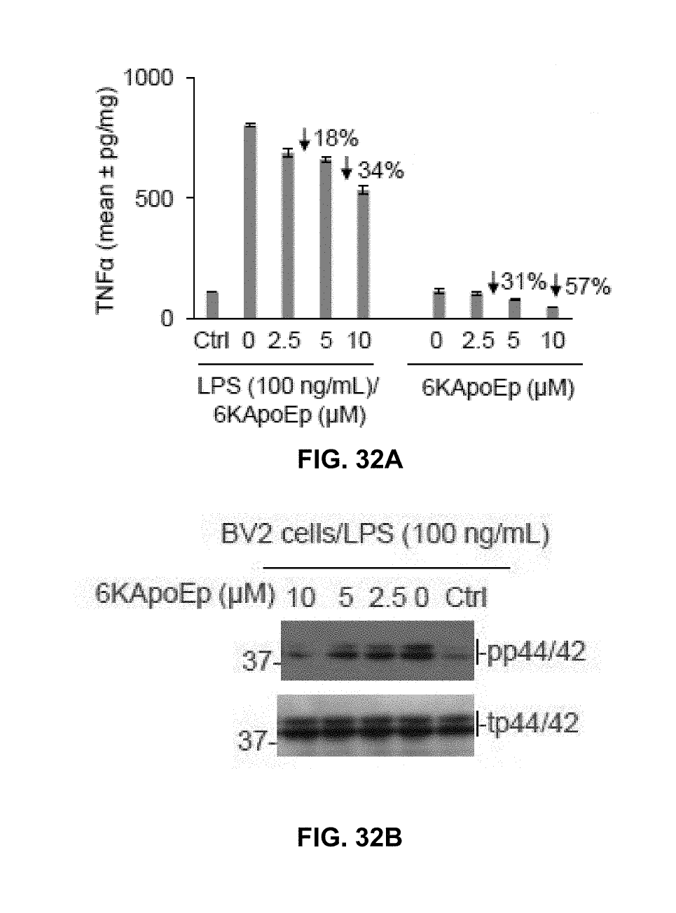

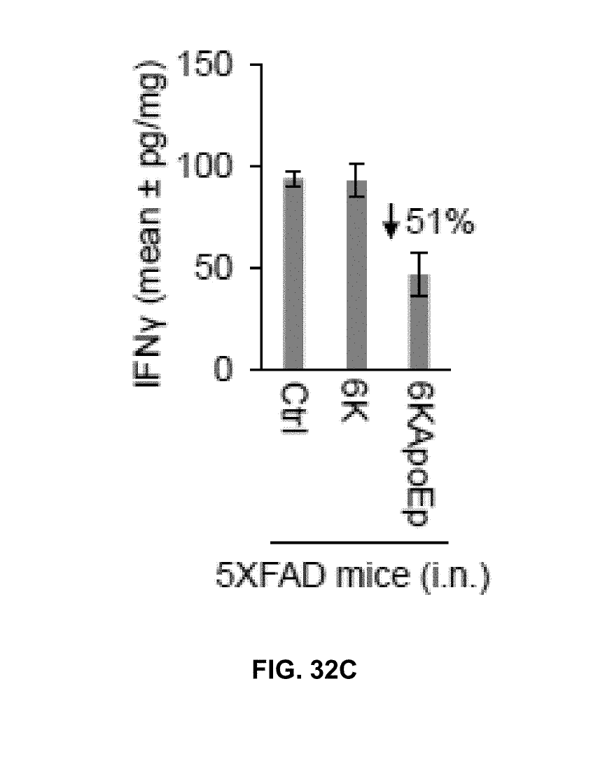

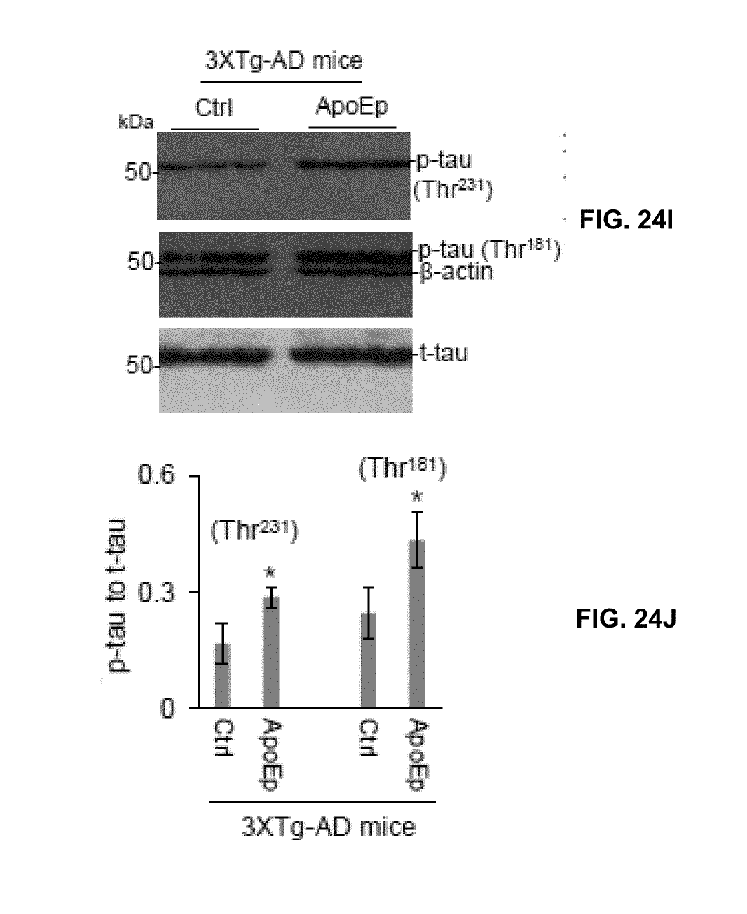

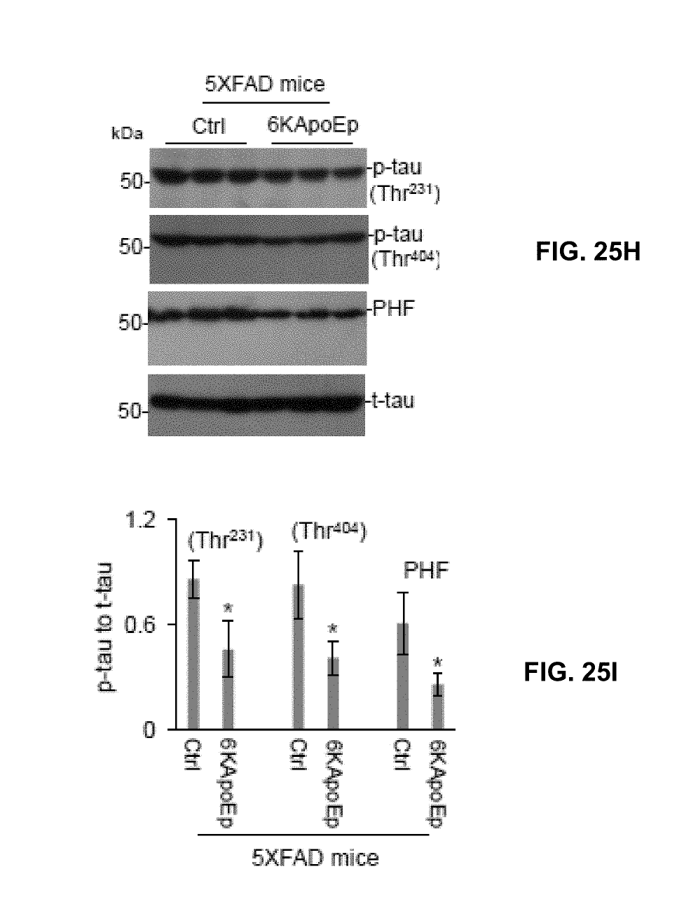

This Example can demonstrate a disease modifying therapeutic benefit of 6KApoEp in a mouse model of AD. Peripheral administration of 6KApoEp for 12 weeks reduced AD pathology in 5×FAD mice, reducing Aβ1-40, 42 levels, Aβ plaques, acetylated and phosphorylated tau and cleaved caspase 3, a protein marker of apoptosis, while enhancing neurogenesis, synaptogenesis and hippocampus-dependent learning and memory (FIGS. 25A-26I). Conversely, ApoEp enhanced Aβ1-40, 42 levels, β-amyloid plaque as well as acetylated and phosphorylated tau in another AD mouse model (3×Tg-AD mice), supporting recent studies indicating that apoE mediates multiple AD pathologies, some of which might be independent of Aβ (FIG. 24A-24J)^{21-23,41}. Notably, peripheral administration of biotin-ApoEp and -6KApoEp increased the levels of these peptides in the brain within 30 min of administration, underscoring that these peptides enter the CNS (data not shown). 6KApoEp elicited its therapeutic effects without altering cell-associated or plasma cholesterol levels or the expression of LDLR, LRP1 or apoE, indicating that this treatment does not dramatically alter cholesterol homeostasis (FIGS. 30A-31C). In addition, 6KApoEp reduced basal and LPS-mediated TNFα production and p44/42 MAPK phosphorylation in cultured microglia (FIGS. 32A-32C), suggesting that 6KApoEp has the additional benefit of reducing neuroinflammation.

This Example can suggest that 6KApoEp reduces AD pathology by reducing the physical interaction of apoE with N-terminal APP. Based on this data and without being bound by theory, it is suggested that apoE might have a dual function in the brain, (1) mediating cholesterol transport into the neuron and thereby promoting neuronal proliferation, differentiation and health ^{16,17} and (2) binding to APP and thereby promoting APP amyloidogenic proteolysis and AD pathology^{32,33}. While the cholesterol transporting function of apoE may function well in the young and healthy brain, the APP proteolytic function of apoE might be expected to be a function of aging and disease. This hypothesis is also based on recent findings that APP has a receptor function like other type 1 transmembrane receptors, which either mediate axon guidance, synaptogenesis and growth factor signaling or AD pathogenesis, depending on the environment ^{25-27,34,35}. For example, overstimulation of APP by apoE might lead to overactivation of Go protein and AICD, which can be pathogenic. In addition, other factors such as apoE lipidation, glycosylation and oxidation could play a role in determining how apoE functions. Clearly, the N-terminal region of APP mediating apoE pathogenic effect needs further mapping and exploration. As can be demonstrated in this Example, deleting the E1 region of APP eliminates ApoEp-mediated but not spontaneous Aβ production, suggesting the importance of the N-terminal region of APP in amyloidogenesis.

The apoE antagonist 6KApoEp can be particularly beneficial for AD patients who are apoE4 carriers. Since apoE4 has a much stronger binding affinity to its receptors compared with apoE2 18, 6KApoEp may specifically counteract the adverse effects of apoE4 by reducing its binding to its receptors. The validity of this approach is supported by previous studies which have shown that carrying the lower affinity apoE2 protects against AD and reduces the accumulation of β-amyloid pathology in the aged brain^{19,20}. Clinically, older apoE2 carriers display superior verbal learning abilities, improved recall memory, faster processing of information, and better test performance¹⁹. A better understanding of roles of apoE isoforms in neuroplasticity and AD as well as their interaction with N-terminal APP and molecular mechanisms may reveal additional approaches for extending brain health span.

Experimental Procedures.

Reagents and Antibodies.

The following reagents were used: human recombinant apoE2, apoE3 and apoE4 proteins (Biovision, Milpitas, Calif.); hamster LDLR siRNA and negative control (Thermo Fisher Scientific, Waltham, Mass.); ApoE LDLR binding domain peptide (ApoE mimetic peptide, ApoEp, aa sequence LRVRLASHLRKLRKRLLRDA (SEQ ID NO: 1)) and ApoEp modified by N-terminal addition of Flag-Tag (FlagApoEp, DYKDDDDKLRVRLASHLRKLRKRLLRDA (SEQ ID NO: 10), Biotin (BiotinApoEp), 3 lysines (3KApoEp), 6 lysines (6KApoEp), 7 lysines (7KApoEp), 8 lysines (8KApoEp), 9 lysines (9KApoEp), 3 aspartates (3DApoEp), scrambled peptide, N-terminal APP31/60 and APP61-90 (GenScript, Piscataway, N.J.); APP β-secretase and γ-secretase inhibitor and LPS (Millipore Sigma, Burlington, Mass.). Antibodies were used as follows: rabbit polyclonal anti-ApoE antibody (ApoE1H4) (ProMab Biotechnologies, Richmond, Calif.); rabbit monoclonal ApoE antibody (mApoE) (Abcam Inc. Cambridge, Mass.); mouse monoclonal anti-Aβ1-16 antibody (6E10, Biolegend, San Diego, Calif.); mouse monoclonal anti-Aβ1-16 antibody (82E1), rabbit polyclonal anti-human sAPPα antibody (2B3) (IBL-America, Minneapolis, Minn.); biotin mouse monoclonal anti-Aβ17-24 antibody (4G8) (BioLegend); rabbit polyclonal anti-C-terminal APP (751-770) antibody (pC-APP), rabbit polyclonal anti-N-terminal APP antibody (pAPP44/61), rabbit polyclonal anti-C-terminal LRP1 antibody (Millipore Sigma); rabbit polyclonal anti-LDLR antibody (Abnova, Walnut, Calif.); phospho-p38 MAPK (Thr180/Tyr182), p38 MAPK, phospho-p44/42 MAPK (Erk1/2) (Thr202/Tyr204) and p44/42 MAPK (Erk1/2) rabbit polyclonal antibodies (Cell Signaling Technology, Danvers, Mass.); phospho-tau (Thr404, Thermo Fisher Scientific), phospho-tau (Thr231) phospho-tau (Thr181) and PHF antibodies (Millipore Sigma), PSD95, synaptophysin, synapsin I, β-tubulin III, cleaved caspase-3, NeuN antibodies (Abcam) and anti-Flag M2 and anti-rabbit magnetic beads (Millipore Sigma). Rabbit monoclonal antibodies against acetylated tau [Lys174 and Lys274] were kindly provided by Dr. Li Gan (Gladstone Institutes, San Francisco, Calif.). Mouse monoclonal anti-ApoE LDLR binding domain antibodies (6H3B5, 3E5D3 and 3E5H6) were generated by ProMab Biotechnologies.

Cell Culture.

CHO cells engineered to express human wild-type APP (CHO/APPwt) or Swedish mutant APP (CHO/APPswe) were obtained. These cells were cultured in 96 or 24 well plates at 4×10⁴ or 2×10⁵ cells/well, respectively, in Dulbecco's modified Eagle's medium (DMEM) with 10% fetal bovine serum, 1 mM sodium pyruvate and 100 U/mL of penicillin/streptomycin (Invitrogen, Thermo Fisher Scientific). In addition, murine N2a cells transfected with APPwt (N2a/APPwt were obtained), SH-SY5Y cells transfected with APPswe and wild-type SH-SY5Y cells (ATCC, Manassas, Va.) were cultured as previously described⁴². Primary hN2TM human neurons (Neuromics, Edina, Minn.) were cultured in hN2TM human neuron culture media and primary HCN2 human neurons (ATCC) were cultured in DMEM with 4 mM L-glutamine adjusted to contain 1.5 g/L sodium bicarbonate, 4.5 g/L glucose, fetal bovine serum (10%).

Cell Surface Protein Expression.

To determine the effect of apoE4 on cell surface expression of APP, LDLR or LRP1, CHO/APPwt cells were treated with apoE4 (10 μg/mL) in the absence or presence of 6KApoEp (10 μM) for 2 h followed by washing 3 times with ice cold PBS, biotinylation with freshly prepared sulfo-NHS-LC-biotin (2.5 mg/mL) dissolved in ice-cold borate buffer, changed twice over 30 min, quenching with NH4CI—PBS-CM and lysis. Cell lysates were then immunoprecipitated using Neutravidin beads and intracellular proteins obtained by IP/Neutravidin depletion while cell surface proteins obtained by IP/Neutravidin precipitation were analyzed for APP, LDLR and LRP1 by WB using 6E10, anti-LDLR and anti-LRP antibodies, respectively. In addition, cellular membrane associated holo APP was also analyzed by flow cytometry and presented as median FL-1-A. CHO/APPwt cells were also cultured in 8-well slide chambers at 5×105/well overnight, followed by treatment with apoE4 (10 μg/mL) in the absence or presence of 6KApoEp (10 μM) or PBS (Ctrl) in serum-free medium for 2 h. These cells were then permeabilized with 0.05% Triton X-100 for 5 min, washed and stained with rabbit anti-APP-C-terminal (pC-APP) or mouse anti-N-terminal APP antibody (22C11) overnight at 4° C. and then with Alexa Fluor 594 Donkey anti-rabbit or Alexa Fluor 488 goat anti-mouse IgG to detect APP signals using an Olympus FV1000 laser scanning confocal microscope.

ELISA.

Aβ1-40, 42 and sAPPα from cell and brain homogenates were detected by Aβ1-40, 42 and sAPPα ELISA kits (IBL-America), strictly following the manufacturer's instructions^{43,44}.

Western Blotting and Immunoprecipitation.

WB analyses were performed as previously described^{43,44}. Briefly, 10% bicine/tris gel containing 8 M urea was used for separation of Aβ1-40, 42 species from brain homogenates or CHO cell conditioned media or lysates, followed by transfer to 0.2 μm pore-size nitrocellulose membrane (Bio-Rad, Hercules, Calif.). The membrane was boiled in PBS for 3 to 5 min before blocking to enhance sensitivity and then incubated with 82E1. For all other proteins detected by WB, 10% tris-SDS gels were used. Densitometry analysis was performed as previously described using a FluorS Multiimager with Quantity One™ software 43. Immunoprecipitation (IP) was performed by first incubating conditioned media or cell lysates with appropriate antibodies and Protein-A/G Mag Sepharose beads (GE Healthcare Life Sciences, Pittsburgh, Pa.) overnight with gentle rocking at 4° C., followed by three washes with binding buffer (50 mM tris, 150 mM NaCl, pH 7.5) and analysis by WB.

Real-Time PCR.

CHO-APPwt cells were cultured in 6 well plates at 1×10⁶ cells/well for 24 h, followed by treatment with 6kApoEp at 10 μM for 2 or 24 h and then total RNA extraction using RNeasy Plus Mini Kit (Qiagen). The purity and concentration of RNA was quantified using Nanodrop 2000c spectrophotometer (Thermo Fisher Scientific). The quantification of target RNAs was performed in a total volume of 50 μL by real-time one-step RT-qPCR reactions (10 ng of RNA, 250 mM forward and reverse primers) using SYBR Green I in a IQ5 multi-color real-time PCR detection system (Bio-Rad), according to manufacturer's instructions. The following RNA primers were designed to selectively amplify and quantify human APP and LDLR and CHO cell β-actin (IDT): 5′-GGTGAGTTTGTAAGTGATGCC-3′ (APP forward, SEQ ID NO: 11), 5′ TCTTCTTCTTCCACCTCAGC-3′ (APP reverse, SEQ ID NO: 12), 5′ AGTGGCGAATGTATCGCCTT-3′ (LDLR-forward, SEQ ID NO: 13), 5′-TCGTGGCGGTTAGTGAAGAG-3′ (LDLR reverse, SEQ ID NO: 14), 5′-ATATCGCTGCGCTCGTTGTC-3′ (β-actin forward, SEQ ID NO: 15), 5′-CTCGAAGTCCAGGGCAACAT-3′ (β-actin reverse, SEQ ID NO: 16).

Cholesterol Quantitation.

CHO/APPwt cells were cultured overnight in 6-well plates at 2×10⁶ cells/well in serum free DMEM, followed by washing 3 times with ice-cold PBS, lysis by sonication in chloroform:isopropanol:IGEPAL CA-630 (7:11:0.1) and centrifugation. The organic phase was transferred to a new tube, air dried at 50° C. to remove chloroform and diluted 10-fold in cholesterol assay buffer for cholesterol determination by flourometric cholesterol quantitation kit according to manufacturer's instructions (MilliporeSigma).

Mice.

All mice were housed and maintained in the Morsani College of Medicine Animal Facility at the University of South Florida (USF), and all experiments were conducted in compliance with protocols approved by the USF Institutional Animal Care and Use Committee. 5×FAD mice [APP KM670/671NL (Swedish), APP 1716V (Florida), APP V7171 (London), PSEN1 M146L and PSEN1 L286V] have been described previously⁴⁵. The mouse line was maintained on a C57BL/B6SJL hybrid genetic background. Due to 5 mutations, 5×FAD mice rapidly develop severe β-amyloid pathology, accumulating high levels of extracellular β-amyloid plaques, neurodegeneration, and cognitive impairments. 3×Tg-AD mice, harboring presenilin-1 (PS1/M146V), APP (KM670/671NL), and tau (P301L) transgenes, progressively develop 1-amyloid and NFT pathology which potentially synergize to accelerate neurodegeneration by 6 months of age 46. 5×FAD mice at 6 weeks of age (n=10, 5 female/5 male) were intraperitoneally (i.p.) treated with 6KApoEp (250 μg/kg in 50 μL PBS) or PBS (50 μL) daily for 12 weeks. 3×Tg-AD mice at 9 months of age (n=6, 3 female/3 male) were i.p. treated with ApoEp (250 μg/kg in 50 μL PBS) or PBS (50 μL) daily for 12 weeks.

After 11-week treatment, 5×FAD mice were subjected to Y Maze, fear conditioning, novel object recognition and open field behavioral testing as described previously^{47,48}. For Y Maze testing, mice were placed in the center of a ‘Y’ shaped maze, consisting of three arms at 120°, and allowed 5 min to freely explore the arms for three consecutive days while recording the number and sequence of arm entries (ANY-maze video software, Stoelting, Wood Dale, Ill.). For fear conditioning, mice were allowed to freely explore a Plexiglass chamber for 3 min and then subjected to an 80 dB white noise for 30 s (conditioned stimulus) followed by a 0.35 mA mild foot shock for 2 sec (unconditioned stimulus), which was repeated 2 min later. After 72 h of training, the mice were returned to the chamber for 6 min without any tone or shock and monitored for freezing behavior to the context. After 96 h, the mice were returned to the chamber again for 3 min after changing contextual cues using a few drops of vanilla extract and then freezing behavior to the CS was monitored for 3 min (cued test). For novel object recognition test, the mice were allowed to habituate in a square-shaped box for 10 min on each of two consecutive days, followed by exposure to two identical objects for 15 min on day 3. On day 5, one of the familiar objects was replaced with a novel object and exploration times of the familiar and novel object was recorded. For open field test, the mice were allowed to freely explore a cubic (1 m×1 m×1 m) box for 10 min, while monitoring times spent exploring the center and periphery as an index of anxiety.

After behavioral testing, all mice were anesthetized with isoflourane (50 mg/kg) (Millipore Sigma), euthanized by bilateral thoracotomy and transcardially perfused with ice-cold physiological saline containing heparin (10 U/mL, Millipore Sigma). Blood was collected and brains were rapidly isolated for biochemical and immunohistochemical analysis. Briefly, one hemisphere was frozen immediately in liquid nitrogen and stored at −80° C., followed by sonication in RIPA buffer (Cell Signaling Technology) containing protease inhibitor and phosphatase inhibitor cocktail (Thermo Fisher Scientific), centrifugation at 14,000 rpm for 1 h at 4° C. and analysis of the superatant by WB. The other hemisphere was placed in 4% paraformaldehyde in PBS for cryostat sectioning. The 25-μm free-floating coronal sections were collected and stored in PBS with 100 mM sodium azide in 24-well plates at 4° C. for immunohistochemistry analysis.

Immunohistochemistry (IHC) and immunofluorescence (IF). Brain sections from 5×FAD and 3×Tg-AD mice were stained with biotin anti-Aβ17-24 monoclonal antibody (4G8), VECTASTAIN Elite ABC Kit (Vector Laboratories, Burlingame, Calif.) and diaminobenzidine substrate, followed by quantitative image analysis of Aβ burden, as described previously^{43,44}. Briefly, images of five sections (150 μm apart) through each anatomic region of interest were captured and a threshold optical density was obtained that discriminated staining from background. Manual editing of each field was used to eliminate artifacts. Data were reported as percentage of immunolabelled area captured (positive pixels divided by total pixels captured) performed by a single examiner (T.M.) blinded to sample identities. In addition, brain coronal sections were also analyzed by IHC staining with anti-acetylated tau (K174, K274) and anti-phosphorylated tau (Thr231, Thr181 and Thr404) antibodies, as well as IF staining with anti-β-tubulin III, -NeuN, -cleaved caspase-3 and -synapsin I antibodies.

Statistical Analysis.

All data were normally distributed. Therefore, in instances of single mean comparisons, Levene's test for equality of variances followed by t-test for independent samples was used to assess significance. In instances of multiple mean comparisons, analysis of variance was used, followed by post-hoc comparison using Bonferonni's method. Alpha levels were set at 0.05 for all analyses. The statistical package for the social sciences release 23.0 (IBM SPSS) was used for all data analysis.

Peptides, Proteins and Cell Lines Used in this Example

Table 1 shows various proteins used in this Example.

TABLE 1

| Protein | Catalog | Source |
|---|---|---|
| human recombinant apoE2 | 4760 | BioVision |
| human recombinant apoE3 | 4696 | BioVision |
| human recombinant apoE4 | 4699 | BioVision |
| apolipoprotein E, human | 16-16-120500 | Athens Research & |
| plasma | | Technology |
| apolipoprotein B, human | 16-16-120200 | Athens Research & |
| plasma | | Technology |
| lipoproteins, low density, | 12-16-120412-TC | Athens Research & |
| human plasma | | Technology |
| human recombinant sAPPβ | Code No. 27732 | Immuno-Biological |
| | | Laboratories |

Table 2 shows various peptides used in this Example.

TABLE 2

| Peptide | Sequence |
|---|---|
| ApoEp | LRVRLASHLRKLRKRLLRDA |
| | (SEQ ID NO: 1) |
| FlagApoEp | DYKDDDDK-LRVRLASHLRKLRKRLLRDA |
| | (SEQ ID NO: 10) |
| 3KApoEp | KKKLRVRLASHLRKLRKRLLRDA |
| | (SEQ ID NO: 2) |
| 6KApoEp | KKKKKKLRVRLASHLRKLRKRLLRDA |
| | (SEQ ID NO: 5) |
| 7KApoEp | KKKKKKKLRVRLASHLRKLRKRLLRDA |
| | (SEQ ID NO: 6) |
| 8KApoEp | KKKKKKKKLRVRLASHLRKLRKRLLRDA |
| | (SEQ ID NO: 7) |
| 9KApoEp | KKKKKKKKKLRVRLASHLRKLRKRLLRDA |
| | (SEQ ID NO: 8) |
| APP31-60 | TCIDTKEGILQYCQEVYPELQITNVVEANQ |
| | (SEQ ID NO: 17) |
| APP61-90 | EPQIAMFCGRLNMHMNVQNGKWDSDPSGTK |
| | (SEQ ID NO: 18) |
| Scrambled | KARLRDVRKLSALRLRLLRH |
| peptide #1 | (SEQ ID NO: 19) |
| Scrambled | ADKLRKLKRRLALKRKLKRKLKVRHS |
| peptide #2 | (SEQ ID NO: 20) |

Table 3 shows various cell lines used in this Example.

TABLE 3

| Cell Line | Culture Medium | Source |
|---|---|---|
| CHO/APPwt | DMEM with 10% FBS | Dr. Sascha Weggen |
| CHO | Ham's F-12 medium with 5% | Dr. Monty Krieger |
| | FBS | |
| CHO/IdlA7 | Ham's F-12 medium with 5% | Dr. Monty Krieger |
| | FBS | |
| SH-SY5Y | DMEM with 10% FBS | ATCC |
| SH-SY5Y/APPswe | DMEM with 10% FBS | Dr Wataru Araki |
| N2a/APPwt, N2a/APPswe | DMEM with 10% FBS | Dr. Huaxi Xu; Dr. Gopal |
| | | Thinakaran |
| N2 ™ | hN2 ™ human neuron culture | Neuromics |
| | media | |
| Human (Down syndrome) | Pluripotent stem cell SFM | ATCC |
| induced pluripotent stem | XF/FF; Stem cell dissociation | |
| (iPS) cells (ATCC ®- | reagent; Dulbecco's | |
| DYP0730) | phosphate buffered saline; | |
| | ROCK inhibitor Y27632; | |
| | DMEM: F-12 medium; Stem | |
| | cell freezing media | |
| Human astrocyte cell line | RPMI-1640 | ATCC |
| (CCF-STTG1) | | |

Table 4 shows various antibodies used in this Example.

TABLE 4

| Antibody | Type | Catalog | Application | Dilution | Source |
|---|---|---|---|---|---|
| 6E10 | Mouse monoclonal | SIG-39321 | IP | 1:100 | Covance Research |
| | | | WB | 1:1,000 | Products |
| 82E1 | Mouse monoclonal | 10323 | WB | 1:1,000 | IBL-America |
| 4G8 | Mouse monoclonal | SIG-39220 | IHC | 1:1,000 | Covance |
| pC-APP [anti-APP | Rabbit polyclonal | 171610 | WB | 1:1,000 | EMD Merck |
| C- | | | IF | 1:1,000 | Millipore |
| terminal (751-770)] | | | | | |
| 22C11 | Mouse monoclonal | MAB348 | IF | 1:500 | EMD Merck |
| | | | | | Millipore |
| Flag | Rabbit monoclonal | F2555 | WB | 1:1,000 | Sigma-Aldrich |
| LDLR | Rabbit polyclonal | PAB8804 | WB | 1:1,000 | Abnova |
| LRP1 | Rabbit polyclonal | L2170 | WB | 1:1,000 | Sigma-Aldrich |
| phospho-p38 | Rabbit monoclonal | 4511s | WB | 1:1,000 | Cell Signaling |
| | | | | | Technology |
| total p38 | Rabbit polyclonal | 9212S | WB | 1:1,000 | Cell Signaling |
| | | | | | Technology |
| phospho-p44/42 | Rabbit monoclonal | 4370s | WB | 1:1,000 | Cell Signaling |
| | | | | | Technology |
| total p44/42 | Rabbit monoclonal | 4695S | WB | 1:1,000 | Cell Signaling |
| | | | | | Technology |
| phospho-tau | Rabbit polyclonal | OPA103143 | WB | 1:1,000 | Thermo Fisher |
| (Ser)404 | | | | | Scientific |
| phospho-tau | Rabbit polyclonal | AB9668 | WB | 1:1,000 | EMD Merck |
| (Thr)231 | | | IHC | 1:200 | Millipore |
| acetylated tau | Rabbit monoclonal | | WB | 1:1,000 | Dr. Li Gan, |
| (lysine)174 | | | IHC | 1:200 | Gladstone |
| | | | | | Institutes |
| acetylated tau | Rabbit monoclonal | | WB | 1:1,000 | Dr. Li Gan, |
| (lysine)274 | | | IHC | 1:200 | Gladstone |
| | | | | | Institutes |
| 5H6B3, 3E5D3, | Mouse monoclonal | | IP | 1:200 | Promab |
| 3E5H6 | | | | | Biotechnologies |
| PSD95 | Rabbit polyclonal | | WB | 1:1,000 | Abcam |
| | | | IHC | 1:100 | |
| synapsin I | Rabbit polyclonal | AB1543P | WB | 1:1,000 | EMD Merck |
| | | | IHC | 1:100 | Millipore |
| β-tubulin III | Mouse monoclonal | 60052 | WB | 1:1,000 | Stemcell |
| | | | IHC | 1:100 | technologies |
| cleaved Caspase-3 | Rabbit polyclonal | 9661L | WB | 1:1,000 | Cell Signaling |
| | | | IHC | 1:100 | Technology |
| NeuN | Mouse monoclonal | MAB377 | WB | 1:1,000 | EMD Merck |
| | | | IHC | 1:8,000 | Millipore |
| synaptophysin | Rabbit polyclonal | | WB | 1:1,000 | EMD Merck |
| | | | IHC | 1:100 | Millipore |
| pAPP44/61 (pN- | Rabbit polyclonal | | WB | 1:1,500 | Millipore Sigma, |
| APP) | anti-N-terminal APP | | IP | 1:100 | MN_NF-07-667 |
| | antibody | | | | |
| ApoE1H4 (human) | Rabbit polyclonal | | WB | 1:1,500 | Promab |
| | anti-apoE antibody | | | | Biotechnologies |
| mApoE (human and | Rabbit monoclonal | | WB | 1:1,500 | Abcam, ab183597 |
| murine) | anti-apoE antibody | | | | |
| anti-N-terminal | Mouse monoclonal | | WB | 1:1,500 | Millipore Sigma |
| APP antibody (2B3) | anti-N-terminal APP | | | | (MABN850) |
| | antibody | | | | |

### REFERENCES FOR EXAMPLE 7

1 Selkoe, D. J. Alzheimer's disease: genes, proteins, and therapy. Physiol Rev 81, 741-766 (2001).
2 Hardy, J. & Selkoe, D. J. The amyloid hypothesis of Alzheimer's disease: progress and problems on the road to therapeutics. Science (New York, N.Y.) 297, 353-356, doi:10.1126/science.1072994 297/5580/353 [pii] (2002).
3 Schenk, D. B. et al. Therapeutic approaches related to amyloid-beta peptide and Alzheimer's disease. Journal of medicinal chemistry 38, 4141-4154 (1995).
4 Selkoe, D. J. et al. The role of APP processing and trafficking pathways in the formation of amyloid beta-protein. Annals of the New York Academy of Sciences 777, 57-64 (1996).
5 LaFerla, F. M., Tinkle, B. T., Bieberich, C. J., Haudenschild, C. C. & Jay, G. The Alzheimer's A beta peptide induces neurodegeneration and apoptotic cell death in transgenic mice. Nature genetics 9, 21-30, doi:10.1038/ng0195-21 (1995).
6 Loo, D. T. et al. Apoptosis is induced by beta-amyloid in cultured central nervous system neurons. Proceedings of the National Academy of Sciences of the United States of America 90, 7951-7955 (1993).
7 Bradt, B. M., Kolb, W. P. & Cooper, N. R. Complement-dependent proinflammatory properties of the Alzheimer's disease beta-peptide. The Journal of experimental medicine 188, 431-438 (1998).
8 Suo, Z. et al. Alzheimer's beta-amyloid peptides induce inflammatory cascade in human vascular cells: the roles of cytokines and CD40. Brain research 807, 110-117 (1998).
9 Hensley, K. et al. A model for beta-amyloid aggregation and neurotoxicity based on free radical generation by the peptide: relevance to Alzheimer disease. Proceedings of the National Academy of Sciences of the United States of America 91, 3270-3274 (1994).
10 Murakami, K. et al. Formation and stabilization model of the 42-mer Abeta radical: implications for the long-lasting oxidative stress in Alzheimer's disease. Journal of the American Chemical Society 127, 15168-15174, doi:10.1021/ja054041c (2005).
11 Lee, V. M. & Trojanowski, J. Q. Progress from Alzheimer's tangles to pathological tau points towards more effective therapies now. Journal of Alzheimer's disease: JAD 9, 257-262 (2006).
12 Rocca, W. A., Amaducci, L. A. & Schoenberg, B. S. Epidemiology of clinically diagnosed Alzheimer's disease. Annals of neurology 19, 415-424, doi:10.1002/ana.410190502 (1986).
13 Elias, M. F., Elias, P. K., Sullivan, L. M., Wolf, P. A. & D'Agostino, R. B. Lower cognitive function in the presence of obesity and hypertension: the Framingham heart study. Int J Obes Relat Metab Disord 27, 260-268, doi:10.1038/sj.ijo.802225 802225 [pii] (2003).
14 Kapogiannis, D. & Mattson, M. P. Disrupted energy metabolism and neuronal circuit dysfunction in cognitive impairment and Alzheimer's disease. Lancet Neurol 10, 187-198, doi:S1474-4422(10)70277-5 [pii] 10.1016/S1474-4422(10)70277-5 (2011).
15 Eisenstein, M. Genetics: finding risk factors. Nature 475, S20-22, doi:10.1038/475S20a (2011).
16 Mahley, R. W. Apolipoprotein E: cholesterol transport protein with expanding role in cell biology. Science (New York, N.Y.) 240, 622-630 (1988).
17 Mahley, R. W., Weisgraber, K. H. & Huang, Y. Apolipoprotein E: structure determines function, from atherosclerosis to Alzheimer's disease to AIDS. J Lipid Res 50 Suppl, S183-188, doi:10.1194/jlr. R800069-JLR200 (2009).
18 Bu, G. Apolipoprotein E and its receptors in Alzheimer's disease: pathways, pathogenesis and therapy. Nature reviews. Neuroscience 10, 333-344, doi:10.1038/nm2620 (2009).
19 Suri, S., Heise, V., Trachtenberg, A. J. & Mackay, C. E. The forgotten APOE allele: a review of the evidence and suggested mechanisms for the protective effect of APOE varepsilon2. Neuroscience and biobehavioral reviews 37, 2878-2886, doi:10.1016/j.neubiorev.2013.10.010 (2013).
20 Benjamin, R. et al. Protective effect of apoE epsilon 2 in Alzheimer's disease. Lancet (London, England) 344, 473 (1994).
21 Wang, C. et al. Gain of toxic apolipoprotein E4 effects in human iPSC-derived neurons is ameliorated by a small-molecule structure corrector. Nature medicine 24, 647-657, doi:10.1038/s41591-018-0004-z (2018).
22 Shi, Y. et al. ApoE4 markedly exacerbates tau-mediated neurodegeneration in a mouse model of tauopathy. Nature 549, 523-527, doi:10.1038/nature24016 (2017).
23 Uddin, M. S. et al. APOE and Alzheimer's Disease: Evidence Mounts that Targeting APOE4 may Combat Alzheimer's Pathogenesis. Molecular neurobiology, doi:10.1007/s12035-018-1237-z (2018).
24 Zhao, N., Liu, C. C., Qiao, W. & Bu, G. Apolipoprotein E, Receptors, and Modulation of Alzheimer's Disease. Biological psychiatry 83, 347-357, doi:10.1016/j.biopsych.2017.03.003 (2018).
25 Deyts, C., Thinakaran, G. & Parent, A. T. APP Receptor? To Be or Not To Be. Trends in pharmacological sciences 37, 390-411, doi:10.1016/j.tips.2016.01.005 (2016).
26 Sosa, L. J. et al. The physiological role of the amyloid precursor protein as an adhesion molecule in the developing nervous system. Journal of neurochemistry 143, 11-29, doi:10.1111/jnc.14122 (2017).
27 Dawkins, E. & Small, D. H. Insights into the physiological function of the beta-amyloid precursor protein: beyond Alzheimer's disease. Journal of neurochemistry 129, 756-769, doi:10.1111/jnc.12675 (2014).
28 Reinhard, C., Hebert, S. S. & De Strooper, B. The amyloid-beta precursor protein: integrating structure with biological function. The EMBO journal 24, 3996-4006, doi:10.1038/sj.emboj.7600860 (2005).
29 Rossjohn, J. et al. Crystal structure of the N-terminal, growth factor-like domain of Alzheimer amyloid precursor protein. Nature structural biology 6, 327-331, doi:10.1038/7562 (1999).
30 Reinhard, C., Borgers, M., David, G. & De Strooper, B. Soluble amyloid-beta precursor protein binds its cell surface receptor in a cooperative fashion with glypican and syndecan proteoglycans. Journal of cell science 126, 4856-4861, doi:10.1242/jcs.137919 (2013).
31 Sarrazin, S., Lamanna, W. C. & Esko, J. D. Heparan sulfate proteoglycans. Cold Spring Harbor perspectives in biology 3, doi:10.1101/cshperspect.a004952 (2011).
32 Hass, S. et al. Physical interaction of ApoE with amyloid precursor protein independent of the amyloid Abeta region in vitro. The Journal of biological chemistry 273, 13892-13897 (1998).
33 Huang, Y. A., Zhou, B., Wemig, M. & Sudhof, T. C. ApoE2, ApoE3, and ApoE4 Differentially Stimulate APP Transcription and Abeta Secretion. Cell 168, 427-441 e421, doi:10.1016/j.cell.2016.12.044 (2017).
34 Copenhaver, P. F. & Kogel, D. Role of APP Interactions with Heterotrimeric G Proteins: Physiological Functions and Pathological Consequences. Frontiers in molecularneuroscience 10, 3, doi:10.3389/fnmol.2017.00003 (2017).
35 Bukhari, H. et al. Small things matter Implications of APP intracellular domain AICD nuclear signaling in the progression and pathogenesis of Alzheimer's disease. Progress in neurobiology 156, 189-213, doi:10.1016/j.pneurobio.2017.05.005 (2017).
36 Ye, S. et al. Apolipoprotein (apo) E4 enhances amyloid beta peptide production in cultured neuronal cells: apoE structure as a potential therapeutic target. Proceedings of the National Academy of Sciences of the United States of America 102, 18700-18705 (2005).
37 Haass, C. & Selkoe, D. J. Cellular processing of beta-amyloid precursor protein and the genesis of amyloid beta-peptide. Cell 75, 1039-1042 (1993).
38 Habib, A., Sawmiller, D. & Tan, J. Restoring Soluble Amyloid Precursor Protein alpha Functions as a Potential Treatment for Alzheimer's Disease. Journal of neuroscience research 95, 973-991, doi:10.1002/jnr.23823 (2017).
39 Eggert, S., Thomas, C., Kins, S. & Hermey, G. Trafficking in Alzheimer's Disease: Modulation of APP Transport and Processing by the Transmembrane Proteins LRP1, SorLA, SorCS1c, Sortilin, and Calsyntenin. Molecular neurobiology, doi:10.1007/sl2035-017-0806-x (2017).
40 Zhang, X. & Song, W. The role of APP and BACE1 trafficking in APP processing and amyloid-beta generation. Alzheimer's research & therapy 5, 46, doi:10.1186/alzrt211 (2013).
41 Zhao, N., Liu, C. C., Qiao, W. & Bu, G. Apolipoprotein E, Receptors, and Modulation of Alzheimer's Disease. Biological psychiatry, doi:10.1016/j.biopsych.2017.03.003 (2017).
42 Rezai-Zadeh, K. et al. Green tea epigallocatechin-3-gallate (EGCG) modulates amyloid precursor protein cleavage and reduces cerebral amyloidosis in Alzheimer transgenic mice. The Journal of neuroscience: the official journal of the Society for Neuroscience 25, 8807-8814, doi:25/38/8807 [pii] 10.1523/JNEUROSCI.1521-05.2005 (2005).
43 Obregon, D. et al. Soluble amyloid precursor protein-α modulates β-secretase activity and amyloid-β generation. Nature communications 3, 777, doi:Artn 777 Doi 10.1038/Ncomms1781 (2012).
44 Zhu, Y. et al. CD45 deficiency drives amyloid-beta peptide oligomers and neuronal loss in Alzheimer's disease mice. The Journal of neuroscience: the official journal of the Society for Neuroscience 31, 1355-1365, doi:10.1523/jneurosci.3268-10.2011 (2011).
45 Oakley, H. et al. Intraneuronal beta-amyloid aggregates, neurodegeneration, and neuron loss in transgenic mice with five familial Alzheimer's disease mutations: potential factors in amyloid plaque formation. The Journal of neuroscience: the official journal of the Society for Neuroscience 26, 10129-10140, doi:10.1523/jneurosci.1202-06.2006 (2006).
46 Oddo, S. et al. Triple-transgenic model of Alzheimer's disease with plaques and tangles: intracellular Abeta and synaptic dysfunction. Neuron 39, 409-421 (2003).
47 Sawmiller, D. et al. Beneficial effects of a pyrroloquinolinequinone-containing dietary formulation on motor deficiency, cognitive decline and mitochondrial dysfunction in a mouse model of Alzheimer's disease. Heliyon 3, e00279, doi:10.1016/j.heliyon.2017.e00279 (2017).
48 Sawmiller, D. et al. Diosmin reduces cerebral Abeta levels, tau hyperphosphorylation, neuroinflammation, and cognitive impairment in the 3×Tg-AD mice. Journal of neuroimmunology 299, 98-106, doi:10.1016/j.jneuroim.2016.08.018 (2016).
49 Tokuda, T. et al. Lipidation of apolipoprotein E influences its isoform-specific interaction with Alzheimer's amyloid beta peptides. The Biochemical journal 348 Pt 2, 359-365 (2000).
50 Krul, E. S. & Tang, J. Secretion of apolipoprotein E by an astrocytoma cell line. Journal of neuroscience research 32, 227-238, doi:10.1002/jnr.490320212 (1992).

We claim:

1. A recombinant polypeptide comprising:
a first polypeptide having an amino acid sequence that is about 90% to about 100% identical to SEQ ID NO:1; and
a second polypeptide consisting of 3, 6, 7, 8, or 9 lysine residues,
wherein the second polypeptide is operatively coupled to the first polypeptide.

2. The recombinant polypeptide of claim 1, wherein the second polypeptide is operatively coupled to the N-terminal amino acid or the C-terminal amino acid of the first polypeptide.

3. The recombinant polypeptide of claim 1, wherein the second polypeptide is directly fused to the first polypeptide at the N-terminus or the C-terminus of the first polypeptide.

4. The recombinant polypeptide of claim 1, wherein the second polypeptide is operatively coupled between any two amino acids of the first polypeptide.

5. The recombinant polypeptide of claim 1, wherein the recombinant polypeptide has an amino acid sequence according to any one of SEQ ID NOs: 2, 5, 6, 7 or 8.

6. The recombinant polypeptide of claim 1, wherein the recombinant polypeptide is an ApoE, ApoEP, or ApoE and ApoEp antagonist.

7. A pharmaceutical formulation comprising:
an amount of a recombinant polypeptide, wherein the recombinant polypeptide comprises
a first polypeptide having an amino acid sequence that is about 90% to about 100% identical to SEQ ID NO:1; and
a second polypeptide consisting of 3, 6, 7, 8, or 9 lysine residues,
wherein the second polypeptide is operatively coupled to the first polypeptide; and
a pharmaceutically acceptable carrier.

8. The pharmaceutical formulation of claim 7, wherein the second polypeptide is operatively coupled to the N-terminal amino acid or the C-terminal amino acid of the first polypeptide.

9. The pharmaceutical formulation of claim 7, wherein the second polypeptide is directly fused to the first polypeptide at the N-terminus or the C-terminus of the first polypeptide.

10. The pharmaceutical formulation of claim 7, wherein the second polypeptide is operatively coupled between any two amino acids of the first polypeptide.

11. The pharmaceutical formulation of claim 7, wherein the recombinant polypeptide has an amino acid sequence according to any one of SEQ ID NOs: 2, 5, 6, 7 or 8.

12. The pharmaceutical formulation of claim 7, wherein the amount of the recombinant polypeptide is an amount effective to act as an ApoE, ApoEP, or ApoE and ApoEp antagonist.

13. A method comprising:
administering an amount of a recombinant polypeptide to a subject in need thereof, wherein the recombinant polypeptide comprises
a first polypeptide having an amino acid sequence that is about 90% to about 100% identical to SEQ ID NO:1; and
a second polypeptide consisting of 3, 6, 7, 8, or 9 lysine residues,
wherein the second polypeptide is operatively coupled to the first polypeptide.

14. The method of claim 13, wherein the second polypeptide is operatively coupled to the N-terminal amino acid or the C-terminal amino acid of the first polypeptide.

15. The method of claim 13, wherein the second polypeptide is directly fused to the first polypeptide at the N-terminus or the C-terminus of the first polypeptide.

16. The method of claim 13, wherein the second polypeptide is operatively coupled between any two amino acids of the first polypeptide.

17. The method of claim 13, wherein the recombinant polypeptide has an amino acid sequence according to any one of SEQ ID NOs: 2, 5, 6, 7 or 8.

18. The method of claim 13, further comprising the step of measuring A3 protein formation.

19. The method of claim 13, wherein the subject in need thereof has or is suspected of having a neurologic disease or disorder.

20. The method of claim 13, wherein the subject in need thereof has or is suspected of having Alzheimer's disease.


SEQUENCE LISTING (20 sequences)

SEQ ID NO: 1 - protein, 20 aa, Artificial Sequence
  polypeptide configured to modulate binding of a protein the an ApoE receptor
LRVRLASHLRKLRKRLLRDA

SEQ ID NO: 2 - protein, 23 aa, Artificial Sequence
  polypeptide configured to modulate binding of a protein the an ApoE receptor
KKKLRVRLASHLRKLRKRLLRDA

SEQ ID NO: 3 - protein, 24 aa, Artificial Sequence
  polypeptide configured to modulate binding of a protein the an ApoE receptor
KKKKLRVRLASHLRKLRKRLLRDA

SEQ ID NO: 4 - protein, 25 aa, Artificial Sequence
  polypeptide configured to modulate binding of a protein the an ApoE receptor
KKKKKLRVRLASHLRKLRKRLLRDA

SEQ ID NO: 5 - protein, 26 aa, Artificial Sequence
  polypeptide configured to modulate binding of a protein the an ApoE receptor
KKKKKKLRVRLASHLRKLRKRLLRDA

SEQ ID NO: 6 - protein, 27 aa, Artificial Sequence
  polypeptide configured to modulate binding of a protein the an ApoE receptor
KKKKKKKLRVRLASHLRKLRKRLLRDA

SEQ ID NO: 7 - protein, 28 aa, Artificial Sequence
  polypeptide configured to modulate binding of a protein the an ApoE receptor
KKKKKKKKLRVRLASHLRKLRKRLLRDA

SEQ ID NO: 8 - protein, 29 aa, Artificial Sequence
  polypeptide configured to modulate binding of a protein the an ApoE receptor
KKKKKKKKKLRVRLASHLRKLRKRLLRDA

SEQ ID NO: 9 - protein, 30 aa, Artificial Sequence
  polypeptide configured to modulate binding of a protein the an ApoE receptor
KKKKKKKKKKLRVRLASHLRKLRKRLLRDA

SEQ ID NO: 10 - protein, 28 aa, Artificial Sequence
  N-terminal FLAG ApoE recombinant polypeptide
DYKDDDDKLRVRLASHLRKLRKRLLRDA

SEQ ID NO: 11 - DNA, 21 bp, Artificial Sequence
  APP forward primer
ggtgagtttgtaagtgatgcc

SEQ ID NO: 12 - DNA, 20 bp, Artificial Sequence
  APP reverse primer
tcttcttcttccacctcagc

SEQ ID NO: 13 - DNA, 20 bp, Artificial Sequence
  LDLR-forward primer
agtggcgaatgtatcgcctt

SEQ ID NO: 14 - DNA, 20 bp, Artificial Sequence
  LDLR reverse primer
tcgtggcggttagtgaagag

SEQ ID NO: 15 - DNA, 20 bp, Artificial Sequence
  Beta-actin forward primer
atatcgctgcgctcgttgtc

SEQ ID NO: 16 - DNA, 20 bp, Artificial Sequence
  Beta-actin reverse primer
ctcgaagtccagggcaacat

SEQ ID NO: 17 - protein, 30 aa, Artificial Sequence
  APP31-60 polypeptide
TCIDTKEGILQYCQEVYPELQITNVVEANQ

SEQ ID NO: 18 - protein, 30 aa, Artificial Sequence
  APP61-90 polypeptide
EPQIAMFCGRLNMHMNVQNGKWDSDPSGTK

SEQ ID NO: 19 - protein, 20 aa, Artificial Sequence
  Scrambled peptide number 1
KARLRDVRKLSALRLRLLRH

SEQ ID NO: 20 - protein, 25 aa, Artificial Sequence
  Scrambled peptide number 2
ADKLRKLKRRLALKRKLKRKLKVRH